(19) United States
(12) Patent Application Publication
(10) Pub. No.: US 2024/0390490 A1
(43) Pub. Date: Nov. 28, 2024

(54) PHARMACEUTICAL COMPOSITIONS FOR THE TREATMENT OF CANCERS

(71) Applicant: Rutgers, The State University of New Jersey, New Brunswick, NJ (US)

(72) Inventor: Dipak K. SARKAR, New Brunswick, NJ (US)

(73) Assignee: Rutgers, The State University of New Jersey, New Brunswick, NJ (US)

(21) Appl. No.: 18/693,752

(22) PCT Filed: Sep. 19, 2022
(86) PCT No.: PCT/US2022/043929

§ 371 Date: Mar. 20, 2024

Related U.S. Application Data
(60) Provisional application No. 63/245,990, filed on Sep. 20, 2021.
(60) Provisional application No. 63/407,093, filed on Sep. 15, 2022.

(51) Int. Cl. A61K 39/395; A61K 31/138; A61K 31/485; A61K 38/08; A61P 35/00

(52) U.S. Cl. CPC A61K 39/3955; A61K 31/138; A61K 31/485; A61K 38/08; A61P 35/00

(57) ABSTRACT

The present application relates to pharmaceutical compositions and to the use of such compositions in the treatment of certain cancers, wherein such compositions positively affect the stress axis and immune system in patients in need of treatment.

### STATEMENT OF GOVERNMENT SUPPORT

This invention was made with government support under grant number R01-CA208632 awarded by the National Institutes of Health. The government has certain rights in this invention.

### FIELD

The present application relates to pharmaceutical compositions, methods of treatment and the use of such compositions in the treatment of certain cancers. The present application describes pharmaceutical compositions and the use of such compositions that positively affect the stress axis and immune system in patients in need of treatment.

### BACKGROUND

Cancer progression is known to be promoted by increased body stress caused by elevated beta-adrenergic and opiodergic nervous system activities. Several studies have identified a strong association between stress axis and immune system abnormalities and increased growth and progression of breast cancers. Other types of cancers that would be expected to have an association between stress axis and immune system abnormalities and increased growth and progression include infantile hemangiomas, glioblastoma, hepatocellular carcinoma, colorectal cancers, melanoma, cutaneous squamous cell carcinoma, pancreatic cancer, oral cancer, and ovarian cancer.

Breast cancer is the second most prevalent cancer after lung cancer among American women. The National Cancer Institute estimated that there would be 41,760 deaths due to breast cancer and 268,300 new cases of breast cancer among American women for the year 2019 (1). Breast cancers are divided into several molecular subtypes: luminal A, luminal B, triple-negative/basal-like, and Her2 type. The prevalence rates of the four subtypes of breast cancer appear to differ by race. For example, the triple-negative/basal type, a type which has a poor prognosis, is more common among younger black women, while the luminal A tumor type, which has the best prognosis of the subtypes, occurs less often among black women than white women (2, 3). Immune response is considered to be an important prognostic factor in the tumor microenvironment of both HER2+ and basal tumors (4). The molecular and cellular natures of the tumor immune microenvironment may influence the disease outcome by altering the balance of suppressive versus cytotoxic responses in the vicinity of the tumor (5). It is known that effective tumor surveillance by the host immune system protects against disease, but chronic inflammation and tumor “immunoediting” have also been implicated in disease development and progression (6). Hence a treatment strategy targeted to both improving tumor-directed cytotoxic responses may lead to improved treatment outcome for all subtypes of breast cancers.

There is an unmet need to develop novel pharmaceutical compositions to inhibit tumor growth that exert both direct effects on cancer cells and induce antitumor immune responses.

### SUMMARY

In accordance with illustrative embodiments, the present disclosure relates to pharmaceutical combinations, methods of treatment and uses utilizing the pharmaceutical combinations, which can affect the growth and progression of cancers in patients in need of treatment.

In a first embodiment, the present disclosure relates to a pharmaceutical combination comprising: at least one MU (μ) opioid receptor (MOR) antagonist; at least one BETA (β) 2-adrenergic receptor (B2AR) antagonist; at least one compound selected from the group consisting of a DELTA (Δ) opioid receptor (DOR) agonist and an immune checkpoint inhibitor (ICI); and at least one pharmaceutically acceptable carrier or diluent.

In a second embodiment, the present disclosure relates to a pharmaceutical combination, comprising: at least one MU (μ) opioid receptor (MOR) antagonist; at least one DELTA (Δ) opioid receptor (DOR) agonist; at least one BETA (β) 2-adrenergic receptor (B2AR) antagonist; and at least one pharmaceutically acceptable carrier or diluent.

In a third embodiment, the present disclosure relates to a pharmaceutical combination comprising at least one MU (μ) opioid receptor (MOR) antagonist; at least one BETA (3) 2-adrenergic receptor (B2AR) antagonist; at least one immune checkpoint inhibitor (IC); and at least one pharmaceutically acceptable carrier or diluent.

In certain embodiments, the at least one MOR antagonist in the pharmaceutical combination is a 14-hydroxydihydromorphinone containing compound. In certain embodiments, the at least MOR antagonist in the pharmaceutical combination is selected from the group consisting of naltrexone, oxymorphone, naloxone, nalophrine, methylnaltrexone, alvimopan, naloxol, naloxegol, naldemedine, nalodeine, samidorphan, nalmefene, levallorphan, nalbuphine, buprenorphine, diprenorphine, and cyclazocine, and salts of any of the foregoing. In certain embodiments, the at least one MOR antagonist in the pharmaceutical combination has at least a 1:2 affinity (Kᵢ) for the μ-opioid receptor over the κ-opioid receptor. In certain embodiments, the at least one MOR antagonist in the pharmaceutical combination has at least a 1:50 affinity (Kᵢ) for the μ-opioid receptor over the Δ-opioid receptor. In certain embodiments, the at least one MOR antagonist in the pharmaceutical combination is naltrexone or a salt thereof. In certain embodiments, the at least one DOR agonist in the pharmaceutical combination is selected from the group consisting of (D-Pen², D-Pen⁵)-enkephalin (DPDPE), leu-enkephalin, met-enkephalin, deltorphin I, deltorphin II, and (D-ALA², D-Leu⁵)-enkephalin (DADLE), and salts of any of the foregoing. In certain embodiments, the at least one DOR agonist in the pharmaceutical combination is DPDPE or a salt thereof. In certain embodiments, the at least one DOR agonist in the pharmaceutical combination has greater than 80% binding at the Δ-opioid receptor. In certain embodiments, the at least one B2AR antagonist in the pharmaceutical combination is a p-hydroxy amine containing compound. In certain embodiments, the at least one β-hydroxy amine containing compound in the pharmaceutical combination is selected from the group consisting of propranolol, IDI-118,551, bucindolol, butaxamine, carteolol, carvediol, labetalol, nadolol, oxprenolol, penbutolol, pindolol, sotalol, and timolol, and salts of any of the foregoing. In certain embodiments, the at least one R-hydroxy amine containing compound in the pharmaceutical combination is propranolol or a salt thereof. In certain embodiments, the at least one MOR antagonist in the pharmaceutical combination is present in an amount from about 1 mg/kg to about 20 mg/kg. In certain embodiments, the at least one DOR agonist in the pharmaceutical combination is present in an amount from about 50 μg/kg to about 150 μg/kg. In certain embodiments, the at least one B2AR antagonist in the pharmaceutical combination is present in an amount from about 1 mg/kg to about 20 mg/kg. In certain embodiments, the at least one MOR antagonist, DOR agonist and B2AR antagonist are present in separate pharmaceutical compositions. In certain embodiments, the at least one MOR antagonist, DOR agonist and B2AR antagonist are present in the same pharmaceutical composition.

In certain embodiments, the at least one ICI is selected from the group consisting of an anti-programmed cell death protein 1 (PD-1) inhibitor, an anti-programmed cell death protein 1 ligand (PD-L1) inhibitor, and a cytotoxic T lymphocyte antigen-4 (CTLA-4) inhibitor. In certain embodiments, the at least one ICI is a PD-1 inhibitor such as, but not limited to, nivolumab (Opdivo), pembrolizumab (Keytruda), cemiplimab (Libtayo), dostarlimab (Jemperli), JTX-4014, spartalizumab (PDR001), camrelizumab (SHR1210), sintilimab (1B1308), tislelizumab (BGB-A317), toripalimab (JS 001), pidilizumab, INCMGA00012 (MGA012), AMP-224, AMP-514, and RMP1-14, and salts of any of the foregoing. In certain embodiments, the at least one ICI is nivolumab. In certain embodiments, the at least one ICI is a PD-L1 inhibitor. In certain embodiments, the PD-L1 inhibitor is an anti-PD-L1 antibody selected from the group consisting of atezolizumab (Tecentriq), avelumab (Bavencio), and durvalumab (Imfizi), and salts of any of the foregoing. In certain embodiments, the at least one ICI is a CTLA-4 inhibitor. In certain embodiments, the CTLA-4 inhibitor is ipilimumab or tremelimumab. In certain embodiments, the pharmaceutical combination comprises two or more immune checkpoint inhibitors (ICIs). In certain embodiments, the pharmaceutical combination comprises a PD-1 inhibitor and a CTLA-4 inhibitor. In certain embodiments, the pharmaceutical combination comprises nivolumab and ipilimumab. In certain embodiments, the at least one ICI is present in an amount from about 1 mg/kg to about 20 mg/kg of body weight, preferably from about 1 to about 5 mg/kg of body weight, and more preferably about 1 mg/kg or about 3 mg/kg of body weight. In certain embodiments, the at least one MOR antagonist, B2AR antagonist and ICI are present in separate pharmaceutical compositions. In certain embodiments, the at least one MOR antagonist, B2AR antagonist and ICI are present in the same pharmaceutical composition.

In a fourth embodiment, the present disclosure relates to a pharmaceutical combination for use in a method of affecting growth and progression of cancer, the combination comprising: at least one MU (μ) opioid receptor (MOR) antagonist; at least one BETA (β) 2-adrenergic receptor (B2AR) antagonist; at least one compound selected from the group consisting of a DELTA (Δ) opioid receptor (DOR) agonist and an immune checkpoint inhibitor (ICI); and at least one pharmaceutically acceptable carrier or excipient.

In a fifth embodiment, the present disclosure relates to a method of treatment for affecting the growth and progression of a cancer in a patient in need thereof, comprising administering to the patient a therapeutically effective amount of a pharmaceutical combination, the combination comprising: at least one MU (μ) opioid receptor (MOR) antagonist; at least one BETA (β) 2-adrenergic receptor (B2AR) antagonist; at least one compound selected from the group consisting of a DELTA (Δ) opioid receptor (DOR) agonist and an immune checkpoint inhibitor (ICI); and at least one pharmaceutically acceptable carrier or diluent. In certain embodiments, the cancer is selected from the group consisting of breast cancer, colorectal cancer, melanoma, infantile hemangiomas, glioblastoma, hepatocellular carcinoma, cutaneous squamous cell carcinoma, pancreatic cancer, oral cancer, and ovarian cancer. In certain embodiments, the cancer is breast cancer. In certain embodiments, the cancer is colorectal cancer. In certain embodiments, the cancer is melanoma.

In a sixth embodiment, the present disclosure relates to a pharmaceutical combination for use in a method of affecting the growth and progression of cancer, e.g., breast cancer, comprising: at least one MU (μ) opioid receptor (MOR) antagonist; at least one DELTA (Δ) opioid receptor (DOR) agonist; at least one BETA (β) 2-adrenergic receptor (B2AR) antagonist; and at least one pharmaceutically acceptable carrier or diluent. In certain embodiments, the pharmaceutical combination affects the growth and progression of breast cancer through at least one physiological pathway selected from the group consisting of increased cell growth arrest, elevated levels of apoptotic proteins, reduced production of epithelial-mesenchymal transition factors in tumor cells, increased innate immune activation, increased infiltration of NK cells in the tumor microenvironment, and the reduced inflammatory cytokine levels in plasma. In certain embodiments, the pharmaceutical combination affects the growth and progression of at least one molecular subtype of breast cancer selected from the group consisting of luminal A, luminal B, triple-negative/basal-like, and Her2 type. In certain embodiments of the pharmaceutical combination, the at least one MOR antagonist is naltrexone, the at least one DOR agonist is DPDPE, and the at least one B2AR antagonist is propranolol.

In a seventh embodiment, the present disclosure relates to a pharmaceutical combination for affecting the growth and progression of cancer, e.g., breast cancer, comprising: at least one MU (μ) opioid receptor (MOR) antagonist; at least one DELTA (Δ) opioid receptor (DOR) agonist; at least one BETA (β) 2-adrenergic receptor (B2AR) antagonist; and at least one pharmaceutically acceptable carrier or diluent, wherein the combination inhibits one or more inflammatory cytokines selected from the group consisting of G-CSF/CSF-3, IL-1 alpha, IL-10, IL-6, IL-5, Gro-α, TNF-α, MCP-3, and IL-17A. In certain embodiments, the cancer is breast cancer.

In an eighth embodiment, the present disclosure relates to a pharmaceutical combination for affecting the growth and progression of cancer, e.g., breast cancer, comprising: at least one MU (μ) opioid receptor (MOR) antagonist; at least one DELTA (Δ) opioid receptor (DOR) agonist; at least one BETA (β) 2-adrenergic receptor (B2AR) antagonist; and at least one pharmaceutically acceptable carrier or diluent, wherein the combination inhibits one or more chemokines selected from the group consisting of RANTES and MCP-1.

In a ninth embodiment, the present disclosure relates to a pharmaceutical combination for affecting the growth and progression of cancer, e.g., breast cancer, comprising: at least one MU (μ) opioid receptor (MOR) antagonist; at least one DELTA (Δ) opioid receptor (DOR) agonist; at least one BETA (β) 2-adrenergic receptor (B2AR) antagonist; and at least one pharmaceutically acceptable carrier or diluent, wherein the combination increases the plasma concentration of cytokines involved in the response of natural killer (NK) cells selected from the group consisting of IL-2, IL-4, IL-13, IFN-γ, and IL-12p70.

In a tenth embodiment, the present disclosure relates to a method of treatment for affecting the growth and progression of a cancer, preferably breast cancer, in a patient in need thereof, comprising: administering to the patient a therapeutically effective amount of a pharmaceutical combination, comprising: at least one MU (μ) opioid receptor (MOR) antagonist; at least one DELTA (Δ) opioid receptor (DOR) agonist; at least one BETA (β) 2-adrenergic receptor (B2AR) antagonist; and at least one pharmaceutically acceptable carrier or diluent. In certain embodiments, the pharmaceutical combination is administered as part of a combination therapy, wherein the combination therapies are selected from the group consisting of surgery, radiation, immunotherapy, and chemotherapy. In certain embodiments, the pharmaceutical combination inhibits one or more inflammatory cytokines selected from the group consisting of G-CSF/CSF-3, IL-1 alpha, IL-10, IL-6, IL-5, Gro-α, TNF-α, MCP-3, and IL-17A. In certain embodiments, the pharmaceutical combination inhibits one or more chemokines selected from the group consisting of RANTES and MCP-1. In certain embodiments, the pharmaceutical combination increases the plasma concentration of cytokines involved in the response of natural killer (NK) cells selected from the group consisting of IL-2, IL-4, IL-13, IFN-γ, and IL-12p70.

In an eleventh embodiment, the present disclosure relates to a pharmaceutical combination for affecting the growth and progression of cancer, the composition comprising at least one MU (μ) opioid receptor (MOR) antagonist; at least one BETA (β) 2-adrenergic receptor (B2AR) antagonist; at least one immune checkpoint inhibitor (ICI); and at least one pharmaceutically acceptable carrier or diluent.

In an twelve embodiment, the present disclosure relates to a method of treatment for affecting the growth and progression of a cancer in a patient in need thereof, comprising administering to the patient a therapeutically effective amount of a pharmaceutical combination, the combination comprising at least one MU (μ) opioid receptor (MOR) antagonist; at least one BETA (β) 2-adrenergic receptor (B2AR) antagonist; at least one immune checkpoint inhibitor (ICI); and at least one pharmaceutically acceptable carrier or diluent.

In certain embodiments, the cancer is colorectal cancer. In certain embodiments, the cancer is melanoma. In certain embodiments, the at least one MOR antagonist is naltrexone, the at least one B2AR antagonist is propranolol, and the at least one ICI is two ICIs, wherein the ICIs are ipilimumab and nivolumab. In certain embodiments, the at least one MOR antagonist, B2AR antagonist and ICI are present in separate pharmaceutical compositions. In certain embodiments, the at least one MOR antagonist, B2AR antagonist and ICI are present in the same pharmaceutical composition.

Other aspects and advantages of the present disclosure are described further in the following detailed description.

### BRIEF DESCRIPTION OF THE FIGURES

Exemplary embodiments are illustrated in referenced figures. It is intended that the embodiments and figures disclosed herein are to be considered illustrative rather than restrictive.

FIGS. 1A-1 are histograms showing the optical density (OD) values of the effects of propranolol (PRO), naltrexone (NTX), and (D-Pen2,5)-enkephalin (DPDPE) alone or in combination on breast cancer cell lines MDA-MB-231, MDA-MB-468, and T47D. FIGS. 1A-C show the clonogenic behavior (colony formation) of MB-231 cells, MDA-MB-468 cells, and T47D cells after 14 days of treatment with a 100 μM dose of PRO, NTX, and DPDPE alone or in combinations of NTX+DPDPE, NTX+PRO, and NTX+DPDPE+PRO. FIGS. 1D-F show the effects of PRO, NTX, and DPDPE alone or in combinations of NTX+DPDPE, NTX+PRO, and NTX+DPDPE+PRO for a period of 12 hours on cell migration of MB-231 cells, MDA-MB-468 cells, and T47D cells (mean±SEM; n=4; number of cells/field). FIGS. 1G-I show the effects on cell invasion of MB-231 cells, MDA-MB-468 cells, and T47D cells after 48 hours of treatment with PRO, NTX, and DPDPE alone or in combinations of NTX+DPDPE, NTX+PRO, and NTX+DPDPE+PRO (mean±SEM from four independent experiments in duplicates). Statistical significance was determined using one-way ANOVA with the Newman-Keuls post hoc test. *, p<0.05, **, p<0.01, ***, p<0.001 compared to control, @, p<0.05, compared with single-drug dose. #, p<0.05, compared with the rest of the groups.

FIGS. 2A-L are western blots and histograms. PRO, NTX, and DPDPE increase G2/M arrest (FIGS. 2A-C) and elevate levels of apoptosis-related proteins (FIGS. 2D-L) in MDA-MB-231, MDA-MB-468, and T47D cells. Cells were treated with a 100 μM concentration of PRO, NTX, and DPDPE alone or vehicle alone for 48 hours. After the treatment period, cells were stained with PI, analyzed for cell cycle distribution using flow cytometry. Proportions of cells in each phase were quantified and the values are shown as histograms (FIGS. 2D-L). Representative western blots are presented on the top and mean densitometric values are presented as ratio of β-actin in the histograms. After the drug treatments, some cells were extracted and used for western blot analysis of Bcl-xL (FIGS. 2D, 2G, 2J), Bcl-2 (FIGS. 2E, 2H, 2I), and CC3 (FIGS. 2DF 2I, 2L). Data presented are mean±SEM (n=5-6 animals/group) and were analyzed using one-way ANOVA with the Newman-Keuls multiple comparisons post hoc test. *, p<0.05, **, p<0.01, ***, p<0.001 compared to control, @, p<0.05, compared with single-drug dose. #, p<0.05, compared with the rest of the groups.

FIGS. 3A-F are immunoblots (FIGS. 3A-C) and graphs (FIGS. 3A-F) showing treatment with PRO, NTX, or DPDPE increased human NK (NK-92 MI) cells functions. NK-92 MI cells (1×10⁶ cells) were treated with drugs alone or in combination or vehicle alone at a concentration of 50 μM for 12 hours and then used for measurements of perforin (FIG. 3A), granzyme B (FIG. 3B) and IFN-γ (FIG. 3C) or cytolytic activity on MDA-MB-231 cells (FIG. 3D), MDA-MB-468 cells (FIG. 3E), and T47D cells (FIG. 3F). Results are expressed as the mean±SEM (n=8). Statistical significance was determined using one-way ANOVA with the Newman-Keuls post hoc test. ***, p<0.001 vs. control. @, p<0.01, compared with single-drug dose.

FIGS. 4A-F are graphs and images showing PRO, NTX, and DPDPE decrease tumor growth and increase survival in MDA-MB-231-cell-derived xenografts in nude rats. FIG. 4A shows changes in the tumor volume following treatments with PRO, NTX, and DPDPE alone or in combinations of NTX+DPDPE, NTX+PRO, and NTX+DPDPE+PRO. Values are mean±SEM (n=8/group). Comparison of tumor volume between groups was done by two-way ANOVA with Bonferroni's multiple comparisons test. ***, p<0.001 vs. control. @, p<0.001, compared with single-drug dose. #, p<0.001, compared with the rest of the groups. FIG. 4B shows changes in gross morphology and size differences off representative excised xenograft tumors from the flanks of control rats and rats treated with PRO, NTX, and DPDPE alone or in combinations of NTX+DPDPE, NTX+PRO, and NTX+DPDPE+PRO. FIG. 4C shows tumor weight (mean±SEM; n=5/group) of control rats and rats treated with PRO, NTX, and DPDPE alone or in combinations of NTX+DPDPE, NTX+PRO, and NTX+DPDPE+PRO. Comparison of tumor weight between groups was analyzed by one-way ANOVA with the Newman-Keuls post hoc test. **, p<0.01, ***, p<0.001 vs. control. #, p<0.001, compared with the rest of the groups. FIG. 4D shows the effects of PRO, NTX, and DPDPE alone or in combinations of NTX+DPDPE, NTX+PRO, and NTX+DPDPE+PRO on survival time (days after tumor cell injection) of nude rats inoculated with MDA-MB-231 cells. Kaplan-Meier survival analysis was used to test significant differences between survival curves and mean survival time for rats from each group. Multiple comparisons of survival curves were done with the Log-rank (Mantel-Cox) test (**** <0.001 compared to control). FIG. 4E are photographs of nude rats injected with MDA-MB 231/Luc-GFP cells and treated with PRO, NTX, and DPDPE alone or in combinations of NTX+DPDPE, NTX+PRO, and NTX+DPDPE+PRO and monitored for a period of 6 weeks by using bioluminescence imaging. Relative intensities of emitted light were represented as a pseudo color image ranging from purple (least intense) to white (most intense), generated in living image and superimposed onto the grayscale reference image. FIG. 4F shows the signal intensity (SI) expressed as photon flux (photo/sec) from the tumor (mean±SEM; n=6/group). Comparison of photon flux between groups was done by two-way ANOVA with Bonferroni's multiple comparisons test. ***, p<0.001 vs. control. @, p<0.001, compared with single drug.

FIGS. 5A-N (are images (FIGS. 5A, 5C, 5E), histograms (FIGS. 5B, 5D, 5F), and western blots and histograms (FIGS. 5G-N) showing treatment with PRO, NTX, or DPDPE, alone or in combination, decreased cell mitosis, cell proliferation, cellular apoptosis, and epithelial-mesenchymal transition (EMT) in tumor xenografts. Drug-treated MDA-MB-231-cell-derived xenografts in nude rats were collected and employed for histological determination of cell mitosis (FIGS. 5A-B); immunohistochemical measurements of Ki67 (FIGS. 5C-D) and cleaved caspase 3 (FIGS. 5E-F); and western blot measurements of Bcl-xL (FIG. 5G), Bcl-2 (FIG. 5H), and cleaved caspase 3 (FIG. 5I); Snail (FIG. 5J), Slug (FIG. 5K), Twist (FIG. 5L); E-cadherin (FIG. 5M), and N-cadherin (FIG. 5N). FIGS. 5A, 5C, 5E show changes in tumor histopathology and protein immunohistochemistry in drug-treated MDA-MB-231-cell-derived xenografts in nude rats, with arrows used to denote the cellular changes and a dotted square used to show the area of intensity). Images in FIGS. 5A, 5C, 5E are represented as 20× magnification with 50 μM scale bars. Histological changes were counted by densitometry and presented in the histograms. Western blot analysis of protein levels were measured by densitometry and presented as ratio of β-actin, a house-keeping protein, in histograms. Data presented are mean±SEM (n=5-6 animals/group) and were analyzed using one-way ANOVA with the Newman-Keuls multiple comparisons test. *, p<0.05, **, p<0.01, *** ≤0.001 compared to control, @, p<0.001, compared with single-drug dose. #, p<0.001, compared with the rest of the groups.

FIGS. 6A-R are western blots and histograms showing PRO and NTX modulate levels of apoptosis-related protein in MDA-MB-231, MDA-MB-468, and T47D cells. Western blots are presented on the top and mean densitometric values are presented as a ratio of β-actin in the histograms below the western blots. FIGS. 6A, 6G, 6M show the levels of anti-apoptotic p-Bcl-xL. FIGS. 6B, 6H, 6N show the levels of p-Bcl2. FIGS. 6C, 6I, 6O show the levels of effector proteins such as p-Bax. FIGS. 6D, 6J, 6P show the levels of pro-apoptotic molecule p-Bim. FIGS. 6E, 6K, 6O show the levels of effector caspase 3. FIGS. 6F, 6L, 6R show the levels of Cytochrome c. Data presented are mean±SEM (n=5-6 samples/group) and were analyzed using one-way ANOVA with the Newman-Keuls multiple comparisons post hoc test. * p<0.05, **, p<0.01, *, p<0.001 compared to control, b, p<0.05, compared with NTX. @, p<0.05, compared with the rest of the groups.

FIG. 7. is a schematic illustration of the proposed mechanism of action of naltrexone and propranolol on induction of apoptosis in human breast cancer cells.

FIGS. 8A-AE: Beta-adrenergic and opioidergic agents enhance immune cell function in breast tumor cells xenografted animals. PBMC samples and spleen samples of these rats were employed for flow cytometric measurements of CD161a⁺ NK cell (FIGS. 8A, 8C), CD11b⁺ monocyte (FIG. 8B), and RT1 B⁺ macrophage cell numbers (FIG. 8D). Both PBMC and spleen samples of these rats were used for NK cell cytolytic activity (FIGS. 8E-F). Spleens of these animals were also used for cytotoxic proteins Granzyme B (GzB) (FIG. 8G), perforin (PFN) (FIG. 8H) and IFN-γ (FIG. 8I). plasma was used for cytokine measurements (FIGS. 8J-AE). Each fluorescence histogram's top panel shows the percentages of immune cells in control and treated rats. Data are shown for isotype control (gray) and specific cell surface marker (blue). The bar graphs (below) show the mean±SEM percentage of immune cells. Plasma samples were used for measurements of cytokines using Luminex Multiplex Immunoassay. Data are expressed as mean±SEM (n=5-6). Statistical significance was determined using one-way ANOVA with the Newman-Keuls multiple comparisons test. *, p<0.05, **, p<0.01, *** <0.001 compared to control. @, p<0.05, compared with single-drug dose. #, p<0.05, compared with the rest of the groups.

FIGS. 9A-D: Beta-adrenergic and opioidergic agents alter the tumor immune microenvironment in breast tumor cells xenografted rats. FIGS. 9A-B Top-representative microscopic pictures of immunocytochemical staining demonstrating infiltration of CD161⁺ NK cells and CD163⁺ macrophage in tumor tissues in various treatment groups. Arrows indicate the staining of NK cells and macrophages. Scale bars are 50-μm. Inset in each figure shows a magnified view of representative NK cells and macrophages. Bottom: Histograms showing numbers of tumor infiltrated NK cells and macrophages quantified from six different fields and presented as mean±SEM (n=5-6 animals/group). FIGS. 9C-D Top: Fluorescence histograms showing the percentages of CD161a-expressing NK cells and MHC class II CRT1B-expressing macrophage from tumor tissues of control and treated rats. Data are shown for isotype control (gray) and specific cell surface marker (blue). Bottom: The bar graphs show the mean±SEM (n=5-6 animals/group). percentage NK cells and macrophages as determined from single-color (parameter) flow cytometry histogram. *, p<0.05, **, p<0.01, *** <0.001 compared to control, @, p<0.05, compared with single-drug treatment. #, p<0.05, compared with the rest of the treatment groups.

FIGS. 10A-G are images and immunoblots. FIGS. 10A-C are inverted microscopic pictures of three breast cancer cell lines MDA-MB-231 (FIG. 10A), MDA-MB-468 (FIG. 10B), and T47D (FIG. 10C) in cultures. FIGS. 10D-F are immunoblots of the breast cancer cell lines MDA-MB-231, MDA-MB-468, and T47D showing μ-opioid receptor (MOR; FIG. 10D), d-opioid receptors (DOR; FIG. 10E), and β2-adrenergic receptor (B2AR; FIG. 10F) in these cells. FIG. 10G are immunoblots B2AR, MOR, or DOR after performing immunoprecipitation (IP) with B2AR on MDA-MB-231 cells. IP results are shown in triplicate. β2-actin is the control protein. Protein lysate is the negative control.

FIGS. 11A-I: Propranolol (PRO), naltrexone (NTX), and (D-Pen2,5)-enkephalin (DPDPE) reduce breast tumor cells viability. MDA-MB-231 cells, MDA-MB-468 cells, and T47D cells were cultured in the presence of the indicated doses of a single drug (NTX, DPDPE, or PRO) or drug combination (NTX+DPDPE, NTX+PRO, or NTX+DPDPE+PRO) or vehicle alone for 24, 48, and 72 hours. Cell viability was analyzed by MTT assay at 24, 48 and 72 hours and data presented as mean±SEM (n=12) in FIGS. 11A-C for MDA-MB-231 cells, FIGS. 11D-F for MDA-MB-468 cells, and FIGS. 11G-I for T47D cells.

FIGS. 12A-1: Beta-adrenergic and opioidergic agents suppress breast tumor cells' clonogenic behavior, cell migration, and invasion. FIGS. 12A-C are images, respectively, of MDA-MB-231, MDA-MB-468, and T47D crystal violet stained cell colonies after 14 days of treatment with a 100 μM dose of PRO, NTX, and DPDPE alone or in combination. FIGS. 12-D-F are images, respectively, of cell migration of MDA-MB-231 cells, MDA-MB-468 cells, and T47D cells, which were stained with 0.5% crystal violet from control or after NTX, DPDPE, PRO, NTX+DPDPE, NTX+PRO, or NTX+DPDPE+PRO treatments. FIGS. 12-G-I are images, respectively, of cell invasion of MDA-MB-231 cells, MDA-MB-468 cells, and T47D cells stained with 0.5% crystal violet after NTX, DPDPE, PRO, NTX+DPDPE, NTX+PRO, or NTX+DPDPE+PRO treatments.

FIGS. 13A-C are figures showing cell cycle analysis of MDA-MB-231 cells (FIG. 13A), MDA-MB-468 cells (FIG. 13B), and T47D (FIG. 13C) cells following treatments with NTX, DPDPE, PRO, NTX+DPDPE, NTX+PRO, or NTX+DPDPE+PRO.

FIGS. 14A-L are western blots and histograms showing increase in apoptotic protein levels in MDA-MB-231 cells (FIGS. 14A, 14D, 14G, 14J), MDA-MB-468 cells (FIGS. 14B, 14E, 14H, 14K), and T47D cells (FIGS. 14C, 14F, 14I, 14L), following treatments with NTX, PRO, or NTX+PRO. Western blots are presented on the top and mean densitometric values are presented as a ratio of β-actin in the histograms below the western blots. FIGS. 14A, 14B, 14CM show the levels of anti-apoptotic Bcl-xL. FIGS. 14D, 14E, 14F show the levels of Bcl2. FIGS. 14G, 14HI, 14I, show the levels of effector protein Bax. FIGS. 14J, 14K, 14L show the levels of effector cleaved caspase 3. Data presented are mean±SEM (n=5-6 samples/group) and were analyzed using one-way ANOVA with the Newman-Keuls multiple.

FIGS. 15A-C are images and graphs showing the effect of a single drug (NTX, PRO, or Anti-PD-1) or a drug combination (NTX+PRO, or NTX+PRO+Anti-PD1) treatment on tumor volume of mice inoculated with CT26 cells. FIG. 15A shows gross morphology and size differences of representative CT26 cell xenograft tumors of control and drug-treated mice. FIG. 15B is a graph of the changes in the tumor volume following drug treatments on male mice. FIG. 15C is a graph of the changes in the tumor volume following drug treatments on female mice. Data presented are mean±SEM (n=3/group). Comparison of tumor volume between groups was done by one-way ANOVA with Bonferroni's multiple comparisons test.

FIGS. 16A-D are graphs showing flow cytometry analysis of CD8+ and CD4+ T cells in spleen from CT26 cell tumors bearing male and female BALB/c mice treated with control or a single drug (NTX, PRO, or Anti-PD-1) or a drug combination (NTX+PRO, or NTX+PRO+Anti-PD1). Flow cytometry was used to quantitate percentage (%) of CD8+ and CD4+ T cells from each group. Data presented are mean±SEM (n=5) of CD8+ and CD4+ T cells (of % of CD45+ cells) from male (FIGS. 16A-B) and female (FIGS. 16C-D) animals from each group. *p<0.05,**p<0.01,***p<0.001,****p<0.0001 compared to control as determined by one-way ANOVA with Newman-Keuls multiple comparisons test.

FIGS. 17A-D are graphs showing flow cytometry analysis of CD8+ and CD4+ T cells in PBMC from CT26 cells tumor bearing male and female BALB/c mice treated with control or a single drug (NTX, PRO, or Anti-PD-1) or a drug combination (NTX+PRO, or NTX+PRO+Anti-PD1). Flow cytometry was used to quantitate percentage (%) of CD8+ and CD4+ T cells from each group. Data presented are mean±SEM (n=5) of CD8+ and CD4+ T cells (of % of CD45+ cells) from male (FIGS. 17A-B) and female (FIGS. 17C-D) animals from each group. *p<0.05,**p<0.01,***p<0.001,****p<0.0001 compared to control as determined by one-way ANOVA with Newman-Keuls multiple comparisons test.

FIGS. 18A-D are graphs showing flow cytometry analysis of CD8+ and CD4′ T cells in CT26 cells tumors (1500-2000 mm³ in size) on male and female BALB/c mice treated with control or a single drug (NTX, PRO, or Anti-PD-1) or a drug combination (NTX+PRO, or NTX+PRO+Anti-PD1). Flow cytometry was used to quantitate percentage (%) of CD8+ and CD4+ T cells from each group. Data presented are mean±SEM (n=5) of CD8+ and CD4+ T cells (of % of CD45+cells) from male (FIGS. 18A-B) and female (FIGS. 18C-D) animals from each group. *p<0.05,**p<0.01,***p<0.001,****p<0.0001 compared to control as determined by one-way ANOVA with Newman-Keuls multiple comparisons test.

FIGS. 19A-B are graphs showing flow cytometry analysis of CD49b⁺ NK cells in CT26 cells tumors (1500-2000 mm³ in size) on male and female BALB/c mice treated with control or a single drug (NTX, PRO, or Anti-PD-1) or a drug combination (NTX+PRO, or NTX+PRO+Anti-PD1). Flow cytometry was used to quantitate percentage (%) of CD49b⁺ NK cells from each group. Data presented are mean±SEM (n=5) of CD49b⁺ NK cells (of % of CD45+cells) from male (FIG. 19A) and female (FIG. 19B) animals from each group. *p<0.05,**p<0.01,***p<0.001,****p<0.0001 compared to control as determined by one-way ANOVA with Newman-Keuls multiple comparisons test.

FIGS. 20A-B are graphs showing flow cytometry analysis of CD49b⁺ NK cells in spleen from CT26 cells tumor bearing male and female BALB/c mice treated with control or a single drug (NTX, PRO, or Anti-PD-1) or a drug combination (NTX+PRO, or NTX+PRO+Anti-PD1). Flow cytometry was used to quantitate percentage (%) of CD49b⁺ NK cells from each group. Data presented are mean±SEM (n=5) of CD49b⁺ NK cells (of % of CD45+cells) from male (FIG. 20A) and female (FIG. 20B) animals from each group. *p<0.05,**p<0.01,***p<0.001,****p<0.0001 compared to control as determined by one-way ANOVA with Newman-Keuls multiple comparisons test.

FIGS. 21A-B are graphs showing the changes in cytolytic activity of NK cells in splenocytes against CT26 cancer cells at the effector target ratio (20:1) from samples of control and drug-treated male and female BALB/C mice with tumor xenografts using Calcein AM assays. FIG. 21A shows the effects on NK cell activity in male mice treated with control or a single drug (NTX, PRO, or Anti-PD-1) or a drug combination (NTX+PRO, or NTX+PRO+Anti-PD1. FIG. 21B shows the effects on NK cell activity in female mice treated with control or a single drug (NTX, PRO, or Anti-PD-1) or a drug combination (NTX+PRO, or NTX+PRO+Anti-PD1). Experiments were performed at least three times independently in duplicates, and results are expressed as the mean±SEM (n=5). Statistical significance was determined using one-way ANOVA with the Newman-Keuls post hoc test. ***, p<0.001 vs control. ****, p<0.0001, compared with single-drug dose and control group.

### DETAILED DESCRIPTION

Presently few cancer drugs are available which exert both direct effects on cancer cells and induce antitumor immune responses to inhibit tumor growth.

### Combinations of MU (μ) Opioid Receptor (MOR) Antagonist, DELTA (Δ) Opioid Receptor (DOR) Agonist, and BETA (β) 2-Adrenergic Receptor (B2AR) Antagonist

It has been surprisingly found that a pharmaceutical composition, comprising at least one MU (μ) opioid receptor (MOR) antagonist; at least one DELTA (Δ) opioid receptor (DOR) agonist; and at least one BETA (β) 2-adrenergic receptor (B2AR) antagonist decreases tumor growth and increase immune surveillance of cancers. In certain embodiments, the MOR antagonist is a compound comprising a 14-hydroxydihydromorphinone moiety. In certain embodiments, the MOR antagonist is naltrexone, oxymorphone, naloxone, nalophrine, methylnaltrexone, alvimopan, naloxol, naloxegol, naldemedine, nalodeine, samidorphan, nalmefene, levallorphan, nalbuphine, buprenorphine, diprenorphine, and cyclazocine, or salts of any of the foregoing. In certain embodiments, the MOR antagonist is naltrexone or a salt thereof. In certain embodiments, the DOR agonist is a peptide, such as, but not limited to, (D-Pen², D-Pen⁵)-enkephalin (DPDPE), leu-enkephalin, met-enkephalin, deltorphin I, deltorphin II, or (D-ALA², D-Leu⁵)-enkephalin (DADLE), or salts of any of the foregoing. In certain embodiments, the DOR agonist is DPDPE. In certain embodiments, the β-hydroxy amine containing compound is propranolol, IDI-118,551, bucindolol, butaxamine, carteolol, carvediol, labetalol, nadolol, oxprenolol, penbutolol, pindolol, sotalol, or timolol, or salts of any of the foregoing. In certain embodiments, the β-hydroxy amine containing compound is propranolol or a salt thereof.

In certain embodiments, targeting opioidergic and adrenergic agents with a combination of at least one MU (μ) opioid receptor (MOR) antagonist; at least one DELTA (Δ) opioid receptor (DOR) agonist; and at least one BETA (β) 2-adrenergic receptor (B2AR) antagonist results in potent anticancer effects. providing a new combinatorial treatment strategy with more clinical treatment modalities for the treatment of cancers.

Naltrexone (NTX) is an opiate antagonist used for drug and alcohol dependence. D-Pen²,D-Pen⁵]-enkephalin (DPDPE) is a delta-opioid receptor agonist and is known to affect the stress axis and immune system functions. Propranolol (PRO) is a B2AR antagonist, a beta-blocker, used to treat high blood pressure, In some embodiments, the MOR antagonist is preferably NTX, the DOR agonist is preferably DPDPE, and the B2AR antagonist is preferably propranolol (PRO).

It has been previously shown that transplantation of neurons producing proopiomelanocortin-derived peptide β-endorphin (BEP) into the hypothalamus in tumor-bearing animals reduces stress hormone levels and suppresses carcinogen-induced mammary tumor growth, malignancy rate, and metastasis in rat animal models (7, 8). In addition, a BEP neuron transplant increases natural killer (NK) cell numbers and activities and elevates levels of anti-inflammatory cytokines and chemokines in circulation and in the tumor microenvironment. Furthermore, it has been shown that the BEP neuron acts via increasing opioid receptor activity and suppressing P2-adrenergic receptor (B2AR) activity (8, 9), indicating that opioid receptors (OR) and B2AR may be involved in BEP neuronal actions on tumor physiology.

Both OR and B2AR are G-protein-coupled receptors (GPCRs), are closely related receptor systems and coexist in many cells, including immune cells and breast tumor cells (10-12). Opioidergic and adrenergic agents also have been shown to alter immune cell functions and breast tumor cell proliferation (11-15). GPCRs are known to heterodimerize with closely related members resulting in the modulation of their functions (13, 14, 16).

In NK cells, two closely related 5-opioid receptors (DOR) and μ-opioid receptors (MOR) are known to heterodimerize to reduce their binding to the ligands. Also in these immune cells, MOR antagonist naltrexone (NTX) increases DOR agonist and (D-Pen²,D-Pen⁵)-enkephalin (DPDPE)-induced cytolytic activity and cytotoxic factor productions (13, 14). In cardiac myocytes, low doses of a DOR agonist leucine-enkephalin inhibit norepinephrine-mediated function (17).

Physical interaction of B2AR and DOR has also been reported by co-immunoprecipitation studies in CHO cells exogenously expressing both receptors (18). Ligand binding studies show that opioidergic and adrenergic agents can bind to their complementary receptors and enhance the activity of other compound types through an allosteric mechanism (19). Several studies have identified a strong association between stress axis and immune system abnormalities and increased growth and progression of breast cancers. Despite this evidence, the complementary physiological activities of combined B2AR and OR agents in regulating immune cell functions and tumor growth have not been studied.

The antitumor efficacy of the B2AR antagonist propranolol (PRO), the MOR antagonist naltrexone (NTX), and the DOR agonist (D-Pen²,D-Pen⁵)-enkephalin (DPDPE) affects the stress axis and immune system functions. The present disclosure demonstrates that propranolol, a beta-blocker used to treat high blood pressure, and naltrexone, an opiate antagonist used for drug and alcohol dependence, when combined with a delta-opioid receptor agonist ((D-Pen²,D-Pen⁵)-enkephalin) produces a marked inhibitory effect on tumor growth while improving survival rate. These antitumor effects resulted from a reduction in tumor cell proliferation, induction of cellular apoptosis and prevention of epithelial-mesenchymal transition of tumor cells, and enhancement of innate immune response. The present application provides a novel metronomic treatment of cancers with a combination of multiple repositioned drugs.

As demonstrated in the Examples in the present disclosure, these drugs, individually or in combination, inhibited cell growth, colony formation, migration, invasion, and cell cycle progression of MDA-MB-231, MDA-MB-468, and T47D at varying degrees in vitro. The antitumor activities of PRO, NTX, and DPDPE were increased when combined all together compared to a single drug or two-drug combination. The vulnerability of these cancer cells to cytotoxicity of natural killer (NK) cells was enhanced more following the triple drug combinations than with a single drug or two-drug treatment. In the MDA-MB-231 cell xenograft nude rat model, these drugs alone, and more potently when combined, reduced tumor growth, and increased animal survivability. The antitumor activities of these drugs were associated with direct cell intrinsic effects including increased cell growth arrest, elevated levels of apoptotic proteins, and reduced production of epithelial-mesenchymal transition factors in tumor cells, as well as effects on innate immune activation, increased infiltration of NK cells in the tumor microenvironment, and reduced inflammatory cytokine levels in plasma. The combined treatments of PRO, NTX, and DPDPE produced impressive antitumor effects, providing a new combinatorial treatment strategy with more clinical treatment modalities.

In certain embodiments, the at least one MOR antagonist, DOR agonist and B2AR antagonist are present in separate pharmaceutical compositions. In certain embodiments, the at least one MOR antagonist, DOR agonist and B2AR antagonist are present in the same pharmaceutical composition. When administered in separate pharmaceutical compositions, the MOR antagonist, DOR agonist and B2AR antagonist can be administered concurrently or sequentially in any order.

### Combinations of MU (μ) Opioid Receptor (MOR) Antagonist, BETA (β) 2-Adrenergic Receptor (B2AR) Antagonist, and Immune Checkpoint Inhibitor (ICI)

It has been surprisingly found that a pharmaceutical composition, comprising at least one MU (μ) opioid receptor (MOR) antagonist; at least one BETA (β) 2-adrenergic receptor (B2AR) antagonist and at least immune checkpoint inhibitor (ICI) decreases tumor growth and increase immune surveillance of cancers.

As demonstrated herein, a combination of immune checkpoint inhibitor (e.g., anti PD1 antibody), a MOR antagonist (e.g., Naltrexone) and a B2AR antagonist (e.g., propranolol) increases more treatment modalities in a colon cancer mouse xenografts model than each agent alone. Balb/c mice (both male and female) were obtained from Taconic Biosciences and injected with 1×10⁶ cells of CT26. Once palpable tumors develop (˜50 mm3), animals were allocated into 6 groups (3 mice/group). Each group were treated daily with either: Control (IgG2a isotype control, 200 μg/kg), Anti-PD-1 mAb (12.5 mg/kg q week IP), NTX (10 mg/kg, subcutaneously, sc), PRO (10 mg/kg, sc), PRO (10 mg/kg; sc)+NTX (10 mg/kg, sc), or Anti-PD-1 mAb (12.5 mg/kg IP, week)+PRO (10 mg/kg, sc)+NTX(10 mg/kg, sc). Mice were monitored every day. Tumor volume and mice weight were measured daily. Animals were euthanized when the tumor reached 2,000 mm³. As shown in Example 10, CT-26 tumor cells in mouse xenografts responded to PRO, NTX and anti-PD1 treatment alone, and significantly potentiation of the drug effects were seen in combination treatment in both male and female mice. Furthermore, the immune environment of the tumor upon euthanasia of animals at the end of experiment was evaluated. For this, the treatment effects on the peripheral (plasma and spleen) immune cell population, and tumor infiltrating immune cell populations were measured. The results show that naltrexone (MOR antagonist) and propranolol (B2AR antagonist) combination treatment was associated with increased NK cell activation both peripherally and in the tumor microenvironment. and increased infiltration of tumors with CD8+ T-cells. Some of these immune parameters showed potentiation of the drug effects when combined with anti-PDX in both male and female recipient colon tumor xenografted mice.

These data indicate that propranolol, a beta blocker used to treat high blood pressure, naltrexone, an opiate antagonist used for drug and alcohol dependence, and anti-PD1 when combined, produce marked inhibitory effects on tumor growth and tumor mass while increasing NK and T cell activities. The data demonstrate a novel treatment with a combination for the treatment of cancer.

In certain embodiments, the at least one MOR antagonist, B2AR antagonist and ICI are present in separate pharmaceutical compositions. In certain embodiments, the at least one MOR antagonist, B2AR antagonist and ICI are present in the same pharmaceutical composition. When administered in separate pharmaceutical compositions, the MOR antagonist, B2AR antagonist and ICI can be administered concurrently, or sequentially in any order.

### MU (μ) Opioid Receptor (MOR) Antagonist

In certain embodiments, the MOR antagonist can be a compound that comprises a 14-hydroxydihydromorphinone moiety, or a salt thereof. In certain embodiments, the MOR antagonist can be a compound comprising a 14-hydroxydihydromorphine moiety, or a salt thereof. In certain embodiments, the MOR antagonist can be a compound comprising a noroxymorphone moiety, or a salt thereof. In certain embodiments, the MOR antagonist is oxymorphone represented by the structure of formula A:

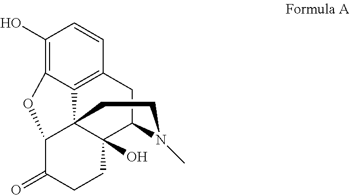

In certain embodiments, the MOR antagonist is a compound represented by the structure of formula B:

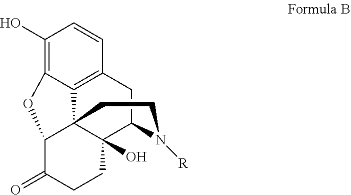

wherein R is alkyl (e.g., methyl), alkenyl (e.g., allyl), cycloalkyl or cycloalkylalkyl (e.g., cyclopropylmethylene or cyclobutylmethylene).

In certain embodiments, the MOR antagonist is naltrexone (17-(cyclopropylmethyl)-4,5α-epoxy-3,14-dihydroxymorphinan-6-one). In certain embodiments, the MOR antagonist is represented by the structure of Formula C:

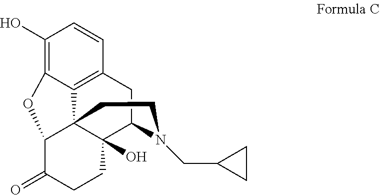

In certain embodiments, the MOR antagonist is a compound such as naltrexone, oxymorphone, naloxone, nalophrine, methylnaltrexone, alvimopan, naloxol, naloxegol, naldemedine, nalodeine, samidorphan, nalmefene, levallorphan, nalbuphine, buprenorphine, diprenorphine, and cyclazocine, and pharmaceutically acceptable salts thereof.

### DELTA (Δ) Opioid Receptor (DOR) Agonist

In certain embodiments, the DOR agonist is a peptide. In certain embodiments, the DOR agonist is (D-Pen², D-Pen⁵)-enkephalin (DPDPE), represented by the structure of formula D:

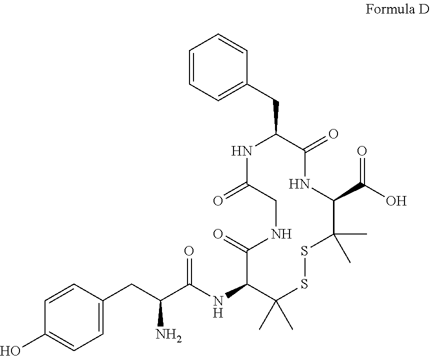 [SMILES: CC1(C)SSC(C)(C)[C@@H](NC(=O)[C@@H](N)Cc2ccc(O)cc2)C(=O)NCC(=O)N[C@@H](Cc2ccccc2)C(=O)N[C@H]1C(=O)O]

In other embodiments, the DOR agonist is a peptide such as leu-enkephalin, met-enkephalin, deltorphin I, deltorphin II, (D-Pen², D-Pen⁵)-enkephalin (DPDPE), or (D-ALA², D-Leu⁵)-enkephalin (DADLE), or salts thereof.

In other embodiments, the DOR agonist is a peptide represented by the structure:

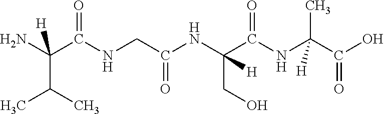 [SMILES: CC(C)[C@H](N)C(=O)NCC(=O)N[C@@H](CO)C(=O)N[C@@H](C)C(=O)O]

### BETA (β) 2-Adrenergic Receptor (B2AR) Antagonist

In certain embodiments, the B2AR antagonist is a β-hydroxy containing compound. The term “β-hydroxy amine”, as used herein, refers to an organic compound having the following moiety where the carbons (*) may be unsubstituted or substituted:

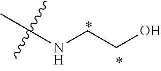 [SMILES: *NCCO]

In certain embodiments, the B2AR antagonist is propranolol, represented by the structure of Formula E, or a salt thereof:

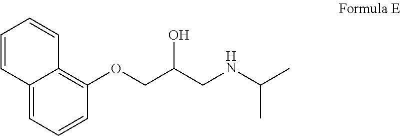 [SMILES: CC(C)NCC(O)COc1cccc2ccccc12]

In certain embodiments, the β-hydroxy amine is a compound such as, for example, propranolol, IDI-118,551, bucindolol, butaxamine, carteolol, carvediol, labetalol, nadolol, oxprenolol, penbutolol, pindolol, sotalol, and timolol, or salts thereof.

### Immune Checkpoint Inhibitors

Immune checkpoint inhibitor (IC) is a type of immunotherapy that block immune checkpoint proteins (e.g., PD1) from binding with partner proteins (e.g., PD-L1). Some tumors produce a lot of PD-L1 to reduce T cell function. Naltrexone and propranolol increase T cell function and possibly prevent the activity of checkpoint proteins. In certain embodiments, an ICI is an anti-programmed cell death protein 1 (PD-1) inhibitor. In certain embodiments, an ICI is an anti-programmed cell death protein 1 ligand (PD-L1) inhibitor. In certain embodiments, an ICI is a cytotoxic T lymphocyte antigen-4 (CTLA-4) inhibitor.

A “Programmed Death-1 (PD-1)” receptor refers to an immunoinhibitory receptor belonging to the CD28 family. PD-1 is expressed predominantly on previously activated T cells in vivo, and binds to two ligands, PD-L1 and PD-L2. The term “PD-1” as used herein includes human PD-1 (hPD-1), variants, isoforms, and species homologs of hPD-1, and analogs having at least one common epitope with hPD-1. A PD-1 inhibitor suitable for use in the combinations and methods of the disclosure can be a biological or chemical compound, such as an organic or inorganic molecule, peptide, peptide mimetic, antibody or an antigen-binding fragment of an antibody. In certain embodiments, the PD-1 inhibitor is an anti-PD-1 antibody. In some further embodiments, the anti-PD-1 antibody is capable of inhibiting binding between PD-1 and PD-L1. In another embodiment, the anti-PD-1 antibody is capable of inhibiting binding between PD-1 and PD-L2. In certain embodiments, a PD-1 inhibitor an anti-PD1 antibody selected from the group consisting of nivolumab (Opdivo), pembrolizumab (Keytruda), cemiplimab (Libtayo), dostarlimab (Jemperli), JTX-4014, spartalizumab (PDR001), camrelizumab (SHR1210), sintilimab (IB1308), tislelizumab (BGB-A317), toripalimab (JS 001), pidilizumab, INCMGA00012 (MGA012), AMP-224, AMP-514, RMP1-14, and salts thereof. In certain embodiments, the PD-1 inhibitor is nivolumab (Opdivo). In certain embodiments, the PD-1 inhibitor is pembrolizumab (Keytruda).

A “Programmed Death Ligand-1 (PD-L1)” is one of two cell surface glycoprotein ligands for PD-1 (the other being PD-L2) that downregulate T cell activation and cytokine secretion upon binding to PD-1. The term “PD-L1” as used herein includes human PD-L1 (hPD-L1), variants, isoforms, and species homologs of hPD-L1, and analogs having at least one common epitope with hPD-L1. A PD-L1 inhibitor suitable for use in the combinations and methods of the disclosure can be a biological or chemical compound, such as an organic or inorganic molecule, peptide, peptide mimetic, antibody or an antigen-binding fragment of an antibody. In certain embodiments, the PD-1 inhibitor is an anti-PD-L1 antibody. In some further embodiments, the anti-PD-L1 antibody is capable of inhibiting binding between PD-1 and PD-L1. In certain embodiments, the PD-1 inhibitor is atezolizumab (Tecentriq), avelumab (Bavencio), or durvalumab (Imfizi).

A “cytotoxic T lymphocyte-associated antigen-4,” “CTLA-4,” refers to an immunoinhibitory receptor belonging to the immunoglobulin (Ig) superfamily. CTLA-4 is a cell surface receptor expressed predominantly on activated T cells, and binds to two ligands, CD80 (B7-1) and CD86 (B7-2). The term “CTLA-4” as used herein includes variants, isoforms, species homologs of human CTLA-4, and analogs having at least one common epitope with CTLA-4. The inhibitor may be an antibody, an antigen binding fragment thereof, an immunoadhesin, a fusion protein or oligopeptide. In certain embodiments, the CTLA-4 inhibitor is ipilimumab. In other embodiments, the CTLA-4 inhibitor is tremelimumab.

In certain embodiments, the pharmaceutical combination comprises two or more immune checkpoint inhibitors. In certain embodiments, the pharmaceutical combination comprises two immune checkpoint inhibitors. In certain embodiments, the pharmaceutical combination comprises three immune checkpoint inhibitors. In certain embodiments, the pharmaceutical combination comprises a PD-1 inhibitor and a CTLA-4 inhibitor. In certain embodiments, the pharmaceutical combination comprises a PD-L1 inhibitor and a CTLA-4 inhibitor. In certain embodiments, the pharmaceutical combination comprises a PD-1 inhibitor and a PD-L1 inhibitor. In certain embodiments, the pharmaceutical combination comprises nivolumab and ipilimumab.

In certain embodiments, the ICI is a PD-1 inhibitor, e.g., nivolumab. In certain embodiments, nivolumab is administered at a dose of about 3 mg/kg. In certain embodiments, nivolumab is administered at a dose of about 3 mg/kg as an intravenous infusion over 60 minutes every 2 weeks. In certain embodiments, nivolumab is supplied as a solution for injection comprising about 10 mg/mL nivolumab in a vial (e.g., 100 mg in 10 mL or 40 mg in 4 mL).

In certain embodiments, the ICI is a CTLA-4 inhibitor, e.g., ipilimumab. In certain embodiments, ipilimumab is administered at a dose of about 3 mg/kg. In certain embodiments, ipilimumab is administered at a dose of about 3 mg/kg every 3 weeks for a maximum of 4 doses. In certain embodiments, ipilimumab is supplied as a solution for injection comprising about 5 mg/mL ipilimumab in a vial (e.g., 50 mg in 10 mL, or 200 mg in 40 mL).

In certain embodiments, the ICI is a combination of nivolumab and ipilimumab. In certain embodiments, ipilimumab is administered at a dose of about 3 mg/kg immediately following nivolumab 1 mg/kg on the same day, every week for 4 doses. Optionally, nivolumab is then administered alone at a dose of about 3 mg/kg as in intravenous infusion.

### Salts

Any compound herein can be provided as a pharmaceutically acceptable salt. Pharmaceutically acceptable salts include, for example, acid-addition salts and base-addition salts. The acid that is added to the compound to form an acid-addition salt can be an organic acid or an inorganic acid. A base that is added to the compound to form a base-addition salt can be an organic base or an inorganic base.

In certain embodiments, a pharmaceutically acceptable salt is a metal salt. Metal salts can arise from the addition of an inorganic base to a compound of the present disclosure. The inorganic base consists of a metal cation paired with a basic counterion, such as, for example, hydroxide, carbonate, bicarbonate, or phosphate. The metal can be an alkali metal, alkaline earth metal, transition metal, or main group metal. In certain embodiments, the metal is lithium, sodium, potassium, cesium, cerium, magnesium, manganese, iron, calcium, strontium, cobalt, titanium, aluminum, copper, cadmium, or zinc. In certain embodiments, a metal salt is a lithium salt, a sodium salt, a potassium salt, a cesium salt, a cerium salt, a magnesium salt, a manganese salt, an iron salt, a calcium salt, a strontium salt, a cobalt salt, a titanium salt, an aluminum salt, a copper salt, a cadmium salt, or a zinc salt. The same can be an ammonium salt, formed from the addition of ammonia or an organic amine to a compound of the present disclosure.

Acid addition salts can arise from the addition of an acid to a compound of the present disclosure. In certain embodiments, the acid is organic. In certain embodiments, the acid is inorganic. In certain embodiments, the salt is a hydrochloride salt, a hydrobromide salt, a hydroiodide salt, a nitrate salt, a nitrite salt, a sulfate salt, a sulfite salt, a phosphate salt, isonicotinate salt, a lactate salt, a salicylate salt, a tartrate salt, an ascorbate salt, a gentisate salt, a gluconate salt, a glucuronate salt, a saccharate salt, a formate salt, a benzoate salt, a glutamate salt, a pantothenate salt, an acetate salt, a propionate salt, a butyrate salt, a fumarate salt, a succinate salt, a methanesulfonate salt, an ethanesulfonate salt, a benzenesulfonate salt, a p-toluenesulfonate salt, a citrate salt, an oxalate salt, or a maleate salt.

### Definitions

The terms used in this specification generally have their ordinary meanings in the art, within the context of this invention and the specific context where each term is used. Certain terms are discussed below, or elsewhere in the specification, to provide additional guidance to the practitioner in describing the methods of the invention and how to use them. Moreover, it will be appreciated that the same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of the other synonyms. The use of examples anywhere in the specification, including examples of any terms discussed herein, is illustrative only, and in no way limits the scope and meaning of the invention or any exemplified term. Likewise, the invention is not limited to its preferred embodiments.

The term “pharmaceutical combination”, as used herein, refers to a therapeutically effective combination of at least one MU (μ) opioid receptor (MOR) antagonist; at least one DELTA (Δ) opioid receptor (DOR) agonist; at least one BETA (β) 2-adrenergic receptor (B2AR) antagonist; and at least one pharmaceutically acceptable carrier or diluent. In other embodiments, term “pharmaceutical combination”, as used herein, refers to a therapeutically effective combination of at least one MU (μ) opioid receptor (MOR) antagonist; at least one BETA (β) 2-adrenergic receptor (B2AR) antagonist; at least one immune checkpoint inhibitor (ICI); and at least one pharmaceutically acceptable carrier or diluent. Optionally, the pharmaceutical combinations of the present disclosure can be used alone or in combination with other suitable therapeutic agents and methods useful in the treatment of cancers including chemotherapeutic agents, anti-tumor agents, anti-inflammatory agents, radiation therapy, and surgery.

The term “subject” as used in this application means an animal with an immune system such as avians and mammals. Mammals include canines, felines, rodents, bovine, equines, porcines, ovines, primates, and humans. Avians include, but are not limited to, fowls, songbirds, and raptors. Thus, the invention can be used in veterinary medicine, e.g., to treat companion animals, farm animals, laboratory animals in zoological parks, and animals in the wild. The invention is particularly desirable for human medical applications.

The term “patient” as used in this application means a human subject.

The term “in need thereof” would be a subject known or suspected of having at least one type of cancer.

The terms “treat”, “treating” or “treatment” of a state, disorder or condition includes: (1) preventing or delaying the appearance of clinical symptoms of the state, disorder, or condition developing in a person who may be afflicted with or predisposed to the state, disorder or condition but does not yet experience or display clinical symptoms of the state, disorder or condition; or (2) inhibiting the state, disorder or condition, i.e., arresting, reducing or delaying the development of the disease or a relapse thereof (in case of maintenance treatment) or at least one clinical symptom, sign, or test, thereof; or (3) relieving the disease, i.e., causing regression of the state, disorder or condition or at least one of its clinical or sub-clinical symptoms or signs.

The term “agent” as used herein means a substance that produces or is capable of producing an effect and would include, but is not limited to, chemicals, pharmaceuticals, biologics, small organic molecules, antibodies, nucleic acids, peptides, and proteins. The terms “agent”, “compound” and “drug” are interchangeable.

The term “about” or “approximately” means within an acceptable error range for the particular value as determined by one of ordinary skill in the art, which will depend in part on how the value is measured or determined, i.e., the limitations of the measurement system, i.e., the degree of precision required for a particular purpose, such as a pharmaceutical formulation. For example, “about” can mean within 1 or more than 1 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated, the term “about” meaning within an acceptable error range for the particular value should be assumed.

As used herein, the terms “therapeutically effective amount”, “therapeutically effective dose” and “effective amount” refer to an amount of the compound and compositions which is sufficient to effect beneficial or desired results, that, when administered alone or in combination with an additional therapeutic agent to a cell, tissue, or subject, is effective to cause a measurable improvement in one or more symptoms of a cancer or in delaying, reducing or mitigating the progression of such cancer. A therapeutically effective dose further refers to that amount of the compound sufficient to result in at least partial amelioration of symptoms, e.g., treatment, healing, prevention or amelioration of the relevant medical condition, or an increase in rate of treatment, healing, prevention or amelioration of such conditions. When applied to an individual active ingredient administered alone, a therapeutically effective dose refers to that ingredient alone. When applied to a combination, a therapeutically effective dose refers to combined amounts of the active ingredients that result in the therapeutic effect, whether administered in combination, serially or simultaneously. An effective amount can also result in an improvement in a subjective measure in cases where subjective measures are used to assess disease severity.

The phrase “pharmaceutically acceptable” or “pharmacologically acceptable” as used herein refers to molecular entities and compositions that are physiologically tolerable and do not typically produce an allergic or similar untoward reaction, such as gastric upset, dizziness and the like, when administered to a human, and approved by a regulatory agency of the Federal or a state government or listed int the U.S. Pharmacopeia or other generally recognized pharmacopeia for use in animals, and more particularly in humans.

The term “carrier” refers to a diluent, adjuvant, excipient, or vehicle with which the therapeutic is administered. Such pharmaceutical carriers can be sterile liquids, such as saline solutions in water and oils, including those of petroleum, animal, vegetable, or synthetic origin, such as peanut oil, soybean oil, mineral oil, sesame oil, and the like. A saline solution is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. The composition, if desired, can also contain minor amounts of wetting or emulsifying agents, or pH buffering agents. The use of solvents, dispersion media, coatings, antibacterial and antifungal agents, isotonic and absorption delaying agents, and the like for pharmaceutical active substances that are pharmaceutically acceptable as the term is used herein are well known in the art and are preferably inert. Except insofar as any conventional media or agent is incompatible with the active ingredients, its use in therapeutic compositions is contemplated.

### Administration and Dosing

The pharmaceutical compositions of the present application can be administered for any of the uses described herein by any suitable means, for example, orally, such as in the form of tablets, capsules, granules or powders; sublingually; bucally; parenterally, such as by subcutaneous, intravenous, intramuscular, or intrasternal injection or infusion techniques (e.g., as sterile injectable aqueous or non-aqueous solutions or suspensions); nasally, including administration to the nasal membranes, such as by inhalation spray; topically, such as in the form of a cream or ointment; or rectally such as in the form of suppositories; in dosage unit formulations containing non-toxic, pharmaceutically acceptable vehicles or diluents. The present pharmaceutical compositions can, for example, be administered in a form suitable for immediate release or extended release. Immediate release or extended release can be achieved using suitable pharmaceutical compositions, or, particularly in the case of extended release, using devices such as subcutaneous implants or osmotic pumps. The present pharmaceutical compositions can also be administered liposomally.

Pharmaceutical compositions adapted for oral administration may be capsules, tablets, powders, granules, solutions, syrups, suspensions (in non-aqueous or aqueous liquids), or emulsions. Tablets or hard gelatin capsules may comprise lactose, starch or derivatives thereof, magnesium stearate, sodium saccharine, cellulose, magnesium carbonate, stearic acid or salts thereof. Soft gelatin capsules may comprise vegetable oils, waxes, fats, semi-solid, or liquid polyols. Solutions and syrups may comprise water, polyols, and sugars. An active agent intended for oral administration may be coated with or admixed with a material that delays disintegration and/or absorption of the active agent in the gastrointestinal tract. Thus, the sustained release may be achieved over many hours and if necessary, the active agent can be protected from degradation within the stomach. Pharmaceutical compositions for oral administration may be formulated to facilitate release of an active agent at a particular gastrointestinal location due to specific pH or enzymatic conditions.

Oral lipid-based drug delivery systems in which a drug is encapsulated or solubilized in lipid excipients can be used to increase solubilization and absorption of a drug, such as a poorly water-soluble drug, to obtain enhanced bioavailability. Various lipid excipients and formulation approaches are described in Kalepu, S. et al., Acta Pharmaceutica Sinica B 2013, 3(6):361-372.

Exemplary compositions for oral administration include suspensions which can contain, for example, microcrystalline cellulose for imparting bulk, alginic acid or sodium alginate as a suspending agent, methylcellulose as a viscosity enhancer, and sweeteners or flavoring agents such as those known in the art; and immediate release tablets which can contain, for example, microcrystalline cellulose, dicalcium phosphate, starch, magnesium stearate and/or lactose and/or other excipients, binders, extenders, disintegrants, diluents and lubricants such as those known in the art. The present pharmaceutical compositions can also be delivered through the oral cavity by sublingual and/or buccal administration. Molded-tablets, compressed tablets or freeze-dried tablets are exemplary forms which may be used. Exemplary compositions include those formulating the present compound(s) with fast dissolving diluents such as mannitol, lactose, sucrose and/or cyclodextrins. Also included in such formulations may be high molecular weight excipients such as celluloses (avicel) or polyethylene glycols (PEG). Such formulations can also include an excipient to aid mucosal adhesion such as hydroxy propyl cellulose (HPC), hydroxy propyl methyl cellulose (HPMC), sodium carboxy methyl cellulose (SCMC), maleic anhydride copolymer (e.g., Gantrez), and agents to control release such as polyacrylic copolymer (e.g., Carbopol 934). Lubricants, glidants, flavors, coloring agents and stabilizers may also be added for ease of fabrication and use.

Exemplary compositions for nasal aerosol or inhalation administration include solutions in saline which can contain, for example, benzyl alcohol or other suitable preservatives, absorption promoters to enhance bioavailability, and/or other solubilizing or dispersing agents such as those known in the art.

Pharmaceutical compositions adapted for parenteral administration, intravenous administration, include aqueous and non-aqueous sterile injectable solutions or suspensions, which may contain antioxidants, buffers, bacteriostats, and solutes that render the compositions substantially isotonic with the blood of the subject. Other components which may be present in such compositions include water, alcohols, polyols, glycerin, and vegetable oils. Compositions adapted for parental administration may be presented in unit-dose or multi-dose containers, such as sealed ampules and vials, and may be stored in a freeze-dried (lyophilized) condition requiring only the addition of a sterile carrier, immediately prior to use. Extemporaneous injection solutions and suspensions may be prepared from sterile powders, granules, and tablets. Suitable vehicles that can be used to provide parenteral dosage forms of the invention are well known to those skilled in the art. Examples include: Water for Injection USP; aqueous vehicles such as Sodium Chloride Injection, Ringer's Injection, Dextrose Injection, Dextrose and Sodium Chloride Injection, and Lactated Ringer's Injection; water-miscible vehicles such as ethyl alcohol, polyethylene glycol, and polypropylene glycol; and non-aqueous vehicles such as corn oil, cottonseed oil, peanut oil, sesame oil, ethyl oleate, isopropyl myristate, and benzyl benzoate.

Exemplary compositions for parenteral administration include injectable solutions or suspensions which can contain, for example, suitable non-toxic, parenterally acceptable diluents or solvents, such as mannitol, 1,3-butanediol, water, Ringer's solution, an isotonic sodium chloride solution, or other suitable dispersing or wetting and suspending agents, including synthetic mono- or diglycerides, and fatty acids, including oleic acid, or Cremaphor.

Exemplary compositions for rectal administration include suppositories which can contain, for example, a suitable non-irritating excipient, such as cocoa butter, synthetic glyceride esters or polyethylene glycols, which are solid at ordinary temperatures, but liquefy and/or dissolve in the rectal cavity to release the drug.

Exemplary compositions for topical administration include a topical carrier such as Plastibase (mineral oil gelled with polyethylene).

It will be understood that the specific dose level and frequency of dosage for any subject can be varied and will depend upon a variety of factors including the activity of the specific compound employed, the metabolic stability and length of action of that compound, the species, age, body weight, general health, sex and diet of the subject, the mode and time of administration, rate of excretion, drug combination, and severity of the condition.

The MU (μ) opioid receptor (MOR) antagonist can be administered orally or intramuscular or subcutaneous injection in an amount of 1 to 20 mg/of body weight, preferably 1 to 10 mg/kg of body weight, and more preferably 10 mg/kg of body weight. Some MOR antagonists (e.g., naltrexone) are not water soluble and often combined with microspheres for intramuscular or subcutaneous injection and extended release. A dose range of 190 to 380 mg naltrexone is used for extended-release compositions. MOR antagonist, naloxone, is used in dose ranges of 0.4 mg/mL, 1 mg/mL, or 2 mg/0.4 mL,

The DELTA (Δ) opioid receptor (DOR) agonist can be administered orally, intramuscular or subcutaneous in an amount of 50 to 150 μg/kg of body weight, preferably 50 to 100 μg/kg of body weight, and more preferably 100 μg/kg of body weight.

The BETA (β) 2-adrenergic receptor (B2AR) antagonist can be administered orally, intravenously or intramuscularly in an amount of 1 to 20 mg/kg of body weight, preferably 1 to 10 mg/kg, and more preferably 10 mg/kg of body weight.

The at least one ICI is present in an amount from about 1 mg/kg to about 20 mg/kg of body weight, preferably from about 1 to about 5 mg/kg of body weight, and more preferably about 1 mg/kg or about 3 mg/kg of body weight.

Selection of a therapeutically effective dose will be determined by the skilled artisan considering several factors, which will be known to one of ordinary skill in the art. Such factors include the particular form of the pharmacological agent, and its pharmacokinetic parameters such as bioavailability, metabolism, and half-life, which will have been established during the usual development procedures typically employed in obtaining regulatory approval for a pharmaceutical compound. Further factors in considering the dose include the condition or disease to be treated or the benefit to be achieved in a normal individual, the body mass of the patient, the route of administration, whether the administration is acute or chronic, concomitant medications, and other factors well known to affect the efficacy of administered pharmaceutical agents. Thus, the precise dose should be decided according to the judgment of the person of skill in the art, and each patient's circumstances, and according to standard clinical techniques.

### EXAMPLES

The present disclosure may be better understood by reference to the following non-limiting examples, which are presented in order to more fully illustrate the preferred embodiments of the invention. They should in no way be construed to limit the broad scope of the invention.

### Materials and Methods

### Breast Cancer Cell Culture:

Human breast cancer cells MDA-MB-231 (Basal B (Claudin-Low); PR⁻, ER⁻, HER2⁻ (Triple Negative)), MDA-MB-468 (Basal A; PR⁻, ER⁻, HER2⁻ (Triple Negative)), and T47D (Luminal A; PR^{+/-}, ER⁺, HER2⁻) were obtained from American Type Culture Collection (ATCC; Rockville, MD), and MDA-MB-231/Luc-GFP cells were purchased from GenTarget Inc. (San Diego, CA; RRID-N/A). MDA-MB-231 cells were cultured in Dulbecco's Modified Eagle Medium (DMEM) with high glucose (Sigma Chemical Co., St Louis, MO), MDA-MB-468 cells were cultured in Leibovitz's L-15 medium (ATCC), and T47D cells were in Roswell Park Memorial Institute (RPMI) 1640 (ATCC) containing bovine insulin (Sigma; 0.2 Units/mL). MDA-MB-231/Luc-GFP cells were purchased from GenTarget Inc., San Diego, CA, and cultured in essential amino acid (NEAA) and 2 mM L-glutamine. All media were supplemented with 10% fetal bovine serum (FBS, Gibco, Gaithersburg, MD) and 1% penicillin-streptomycin (Gibco). Cells were maintained in the cultures at 37′C in a humid environment containing 5% CO₂. Using STR profile authenticated cell lines, it was first confirmed that these human breast cancer cells maintained their morphological phenotype and expressed primary marker receptors (ER, PR, and HER2) during passages as described elsewhere (22).

### Drugs:

Naltrexone (NTX), DPDPE ((D-Pen2,5)-enkephalin), and propranolol (PRO) were all purchased from Sigma, each was dissolved in sterile water as a stock solution at a 50 mM concentration and was stored in aliquots at −20° C. Desired working concentrations of each compound were prepared freshly in the respective media except where specified in the individual experiment. Dilutions represent final concentrations, and an equivalent volume of media was added to wells and serve as background control wells. For animal studies, drugs were dissolved in sterile saline for injection.

### Immunoprecipitation and Immunoblotting Analysis of Beta-Adrenergic and Opioid Receptors:

For immunoprecipitation, a Pierce Classic IP Kit (Thermo Scientific) was used. One mg of cell lysate was precleared using the Control Agarose Resin (14). Lysates were then solubilized in lysis buffer before incubation with anti-B2AR antibody (Abcam) overnight at 4° C. Following the incubation, the immune (antibody/lysate) complex was captured with 20 μl of Pierce Protein A/G Agarose for 1 hour. After elution using the sample buffer, the immune complex was analyzed by western blot analyses with anti-MOR and anti-DOR antibodies (Millipore) and β-actin as a normalizing protein. Experiments were performed in triplicate. Negative control was provided in the kit. Antibody information is given in Table 1.

TABLE 1
List of antibodies used for immunocytochemistry and Western blot analysis &
cell lines used in this study

| REAGENT or RESOURCE | Dilution | SOURCE | IDENTIFIER |
|---|---|---|---|
| Snail (C15D3) Rabbit mAb | 1:250 | Cell Signaling | Cat# 3879, |
| antibody | | Technology, Danvers, | RRID:AB_2255011 |
| | | MA | |
| Mouse Anti-SLUG Monoclonal | 1:250 | Santa Cruz | Cat# sc-166476, |
| Antibody, Unconjugated, Clone | | Biotechnology, | RRID:AB_2191897 |
| A-7 | | Dallas, Texas | |
| Twist (Twist2C1a) antibody | 1:250 | Santa Cruz | Cat# sc-81417, |
| | | Biotechnology, | RRID:AB_1130910 |
| | | Dallas, Texas | |
| Rabbit Anti-E Cadherin | 1:1000 | Abcam, Branford, CT | Cat# ab53033, |
| Polyclonal Antibody, | | | RRID:AB_868611 |
| Unconjugated | | | |
| Rabbit Anti-N Cadherin | 1:1000 | Abcam, Branford, CT | Cat# ab18203, |
| Polyclonal Antibody, | | | RRID:AB_444317 |
| Unconjugated | | | |
| Anti-Bcl-XL antibody | 1:500 | Abcam, Branford, CT | Cat# ab32370, |
| | | | RRID:AB_725655 |
| Anti-Bcl-2 antibody | 1:500 | Abcam, Branford, CT | Cat# ab194583, |
| | | | RRID:AB_2783814 |
| Cleaved Caspase-3 (Asp175) | 1:5000 | Cell Signaling | Cat# 9664, |
| (5A1E) Rabbit mAb antibody | | Technology, | RRID:AB_2070042 |
| | | Danvers, MA | |
| IFN-gamma (G-30) antibody | 1:500 | Santa Cruz | Cat# sc-57208, |
| | | Biotechnology, | RRID:AB_783952 |
| | | Dallas, Texas | |
| Granzyme B (2C5) antibody | 1:500 | Santa Cruz | Cat# sc-8022, |
| | | Biotechnology, | RRID:AB_2232723 |
| | | Dallas, Texas | |
| Perforin 1 (E-5) antibody | 1:500 | Santa Cruz | Cat# sc-374346, |
| | | Biotechnology, | RRID:AB_10988266 |
| | | Dallas, Texas | |
| Ki67 antibody—Proliferation | 1:500 | Abcam, Branford, CT | Cat# ab15580, |
| Marker | | | RRID:AB_443209 |
| β-Actin (8H10D10) Mouse mAb | 1:5000 | Cell Signaling | Cat# 3700, |
| antibody | | Technology, Danvers, | RRID:AB_2242334 |
| | | MA | |
| MOUSE ANTI RAT CD161 | 1:500 | Bio-Rad, Philadelphia, | Cat# MCA1427, |
| antibody | | PA | RRID:AB_2234351 |
| MOUSE ANTI RAT CD163 | 1:500 | Bio-Rad, Philadelphia, | Cat# MCA342R, |
| antibody | | PA | RRID:AB_321966 |
| Anti-μ-opioid receptor antibody | 1:1000 | Millipore, Danvers, MA | Cat# AB_5511 |
| | | | RRID:AB_177512 |
| Anti-δ-opioid receptor antibody | 1:1000 | Millipore, Danvers, MA | Cat# AB_1560 |
| | | | RRID:AB_90778 |
| Anti-β-adrenergic receptor | 1:1000 | Abcam, Branford, CT | Cat# Ab61778 |
| antibody | | | RRID:AB_940566 |
| MDA-MB-231 cells | | ATCC; Rockville, MD | Cat# HTB-26, |
| | | | RRID:CVCL_0062 |
| MDA-MB-468 | | ATCC; Rockville, MD | Cat# HTB-132, |
| | | | RRID:CVCL_0419 |
| T47D cells | | ATCC; Rockville, MD | Cat# HTB-133, |
| | | | RRID:CVCL_0553 |
| MDA-MB-231/Luc-GFP cells | | GenTarget Inc., San | Cat# SC044, |
| | | Diego, CA | RAID-N/A |
| NK cells sensitive YAC-1 cells | | ATCC; Rockville, MD | Cat# TIB-160, |
| | | | RRID:CVCL_2244 |
| Human NK cell line NK-92 MI | | ATCC; Rockville, MD | Cat# CRL-2408, |
| | | | RRID:CVCL_3755 |

### Cell Viability, Colony Formation, Migration, and Invasion Assays Cytotoxicity Assay:

For cytotoxicity assays, cells (MDA-MB-231, MDA-MB-468, and T47D) were plated in 96-well plates (2,000 cells/well), allowed to adhere overnight, and treated with the individual (NTX, DPDPE, or PRO) or drug combination (NTX+DPDPE, NTX+PRO, or NTX+DPDPE+PRO) with increasing concentrations (0.001 μM to 200 μM) for 24, 48, and 72 hours in quadruplicate wells. Cells in media with vehicle treatment are considered control wells. MTT (3-(4,5-dimethyl-2-thiazolyl)-2,5-diphenyl-2H-tetrazolium bromide) was prepared as a stock solution of 5 mg/mL in 1× phosphate-buffered saline (PBS, pH 7.2). At the end of the drug treatment period (24 or 48 or 72 hours), 10 μL of MTT solution was added to each well. After incubation for 4 hours at 37° C., 100 μL of solubilizing buffer (10% sodium dodecyl sulfate dissolved in 0.01 N HCl) was added to each well. After a 15-minute incubation at 37° C., the 96-well plate was read by a plate reader at 590 nm for absorbance density values to determine the cell viability. The cell viability (percentage of control) is calculated by using the following formula: (OD of treated well (−blank))/(mean OD control well (−blank))×100. The values of the inhibitory concentration required to arrest 50% of the cells (IC50) were calculated for individual or drug combinations. Drug effects on these cells were determined in quadruplicate wells in three independent experiments.

To determine whether the NTX+PRO combination (1:1 ratio) has synergistic, additive or antagonistic activity on cell lines, we used isobologram and combination-index methods, as per the principle of Chou and Talalay (24) using the CalcuSyn software (Biosoft, Ferguson, MO). Data obtained from the growth inhibitory experiments (MTT assay) were used to perform these analyses. The isobologram method is a graphical representation of the pharmacologic interaction and is formed by selecting a desired fractional cell kill (Fa) and plotting the individual drug doses required to generate that Fa on their respective x- and y-axes. A straight line is then drawn to connect the points. The observed dose combination of the two agents that achieved that particular Fa is then plotted on the isobologram. The combination-index (Cl) method is a mathematical and quantitative representation of a two-drug pharmacologic interaction. Using data from the growth inhibitory experiments and computerized software, Cl values are generated over a range of Falevels from 0.05 to 0.90 (5%-90% growth inhibition). If the Cl values are <1 it means the drugs have a synergistic effect. If Cl values are equal to 1 it means the drugs have additive effect, or if Cl values are >1 it means the drugs have antagonistic activity.

For other in vitro experiments (clonogenic assay, cell migration, and cell invasion), a drug concentration of 100 μM was chosen for individual or drug combinations. Drug effects on these cells were determined in quadruplicate wells in three independent experiments.

### Assessment of Cell Clonogenicity:

The clonogenic assay was performed as previously described (25). Briefly, cells were suspended in culture media and were seeded into 6-well tissue culture plates at a density of 1,000 cells/well, allowed to adhere overnight, and treated with vehicle alone or a 100 μM concentration of beta-adrenergic and opioidergic drugs alone or in combination. Fresh medium and drugs were changed every 3 or 4 days for 14 days. At the end of the experiment, the cell colonies were stained with 0.5% crystal violet, subjected to image acquisition, and quantified in Adobe Photoshop (Adobe Systems, San Jose, CA). The crystal violet-stained cell colonies were extracted with 10% acetic acid and used for optical density (OD) measurements for determination of colony growth. Drug effects on these cells were determined in duplicate wells in three independent experiments.

### Cell Migration Assay:

Cell migration assay was performed following the procedure described previously (26). In brief, the upper insert chambers (8-μm pore polycarbonate membrane) were seeded with cells at a density of 5×10⁴ cells (cell numbers established by preliminary experiments) per well in the appropriate serum-free medium. A 10% FBS-containing media was then added to the lower chamber to serve as a chemoattractant. A 100 μM concentration of beta-adrenergic and opioidergic drugs alone or in combination or vehicle alone were added to both lower and upper chambers, and cells were allowed to migrate across the membrane for 12 hours, at which point the cells were fixed in 100% methanol for 30 minutes and stained with 0.5% crystal violet for calculating the number of migrated cells in each group. Cells were visualized and photographed using a Nikon Eclipse TE 2000 U motorized inverted microscope (Nikon Corp., Tokyo, Japan) with a Cool SNAP-pro CCD camera. The photographs were used to count the number of migrated cells in Adobe Photoshop.

### Cell Invasion Assay:

The invasive and metastatic potential of cell lines was tested by transmigration through an extracellular matrix (ECM) by a previously described method (26). In brief, the upper chamber of the matrix in 24-well Matrigel invasion chamber plates (BD BioCoat™ Matrigel™ Chamber, BD Biosciences, San Jose, CA) was loaded with 5×10⁴ cells in 500 μl of the appropriate medium (cell numbers established by preliminary experiments). After 24 hours, medium containing 1% FBS was used on both the lower and upper surfaces. One day later, the medium of the upper chamber was replaced with 500 μl medium containing 1% FBS without any drug treatment. The medium was replaced with medium containing 10% FBS and 25 ng/ml EGF in the lower chamber. The effect of beta-adrenergic and opioidergic drugs was tested by adding a 100 μM concentration of these drugs alone or in combination or vehicle alone in both lower and upper chambers. After 48 hours of incubation, the noninvasive cells that remained within the inserts were removed with a cotton swab. Cells that traversed through the Matrigel and the polycarbonate filter (8-μm pore size) attached to the lower surface were stained with 0.5% crystal violet for calculating the invasion index. Cells were visualized and photographed as described in the migration assay. The photographs were used to count invasive cells in Adobe Photoshop.

### Cell Cycle Analysis:

Cell cycle analysis of breast cancer cells was carried out using propidium iodide (PI) staining protocol according to the manufacturer's instructions (Nexcelom Bioscience LLC, Lawrence, MA). Briefly, breast cancer cells were seeded at a density of 1×10⁶ in 10 cm² culture dishes and grown to 50% confluence and were treated with a 100 μM concentration of beta-adrenergic and opioidergic drugs alone or in combination or vehicle alone for 48 hours. Then, cells were harvested by trypsinization, centrifuged at 2000 rpm for 5 minutes, washed with PBS (pH 7.4), and fixed in ice cold 100% ethanol for 15 minutes. The cells were spun down and resuspended in 150 μl of PI-staining solution provided with the Cellometer PI Cell Cycle Kit. The PI-stained cells were then subjected to analysis in the Cellometer Vision instrument (Nexcelom Bioscience LLC) and the data was captured on Cellometer Vision CBA software (Nexcelom Bioscience LLC) and analyzed by FCS Express 4 software (Molecule Devices, Corp., Sunnyvale, CA). Differences in fluorescence intensity were used to determine the percentage of cells in each phase of the cell cycle and represented as histograms for control and drug-treated samples.

### Culture of Human NK Cell Line and Cell-Mediated Cytotoxicity Assay:

The human NK cell line NK-92 MI (ATCC) was maintained in alpha minimum essential medium (MEM) without ribonucleosides and deoxyribonucleosides (Gibco) supplemented with final concentrations of 12-5% FBS, 12-5% horse serum (ATCC), 2 mM L-glutamine (Gibco), 1.5 g/L sodium bicarbonate (Gibco), 0.2 mM inositol (Sigma), 0.1 mM 2-mercaptoethanol (Gibco), and 0.02 mM folic acid (Sigma). For drug treatment, expanded NK-92 MI cells (1×10⁶ cells) were pretreated with a single (NTX, DPDPE or PRO), double combination (NTX+DPDPE or NTX+PRO) or triple combination (NTX+DPDPE+PRO) drugs at a concentration of 50 μM for 12 hours and then used for cytolytic functional assay or Western blot to quantify the level of cytolytic marker proteins such as perforin, granzyme B and IFN-γ as described below. In a pilot cytolytic functional assay, it was determined that the effective dose of each drug is 50 μM (data not shown) and was used for further experiments. The cytolytic function of NK-92 MI cells on breast cancer cells was evaluated by calcein-acetoxymethyl (AM) ester release assay. Briefly, 5 mM calcein-AM (Molecular Probes, OR) labeled breast cancer cell lines (1×10⁵ cells) were allowed to attach overnight in a 96-well plate and were co-incubated with drug pre-treated NK-92 MI cells for 24 hours at an effector (E) and target (T) ratio of 20:1. After centrifugation at 1500 rpm for 3 minutes, the medium from the individual well was transferred to another 96-well plate. The released extracellular calcein-AM (due to lysis of cancer cells) was quantified in a fluorescence microplate reader with the excitation/emission wavelengths of 490/515 nm (Tecan, Mannedrof, Switzerland). The cytotoxicity of NK-92 MI cells on cancer cells was calculated by the formula, percentage (%) of cytolytic activity=((experimental well)−(spontaneous well))/((max lysed well)−(spontaneous well))×100. The percentages at the E:T ratio (20:1) were converted and represented as lytic units (LU).

### Animal Maintenance:

T-cell-deficient, athymic nude (Crl: NIH-Foxn1^{mu}) female rats aged 21-28 days old were purchased from Charles River (Charles River, Portage, MI) and maintained in a pathogen-free condition with a 12-hour light/dark cycle at the institute's animal research facility. Animal care and treatment were performed in accordance with institutional guidelines, and protocols were approved by the Rutgers Institutional Animal Care and Facilities Committee and complied with National Institutes of Health policy.

### Subcutaneous Xenograft Experiments:

For the following study and survival study, MDA-MB 231 cells (ATCC) were grown until about 90% confluence just 1 day before injection. On the day of injection, after checking cell viability with trypan blue, MDA-MB 231 cells were diluted in PBS at a final concentration of 1×10⁷ cells/rat in 200-μl of PBS-50% Matrigel (BD Biosciences, San Jose, CA) mixture and injected subcutaneously (SC) into the right flank of the rats. After tumors reached a diameter of approximately 50 mm³, the animals were randomly assigned to different treatment groups and injected s.c. daily with saline (control) or NTX (10 mg/kg) or DPDPE (100 μg/kg) or PRO (10 mg/kg) or a combination of NTX (10 mg/kg)+DPDPE (100 μg/kg) or NTX (10 mg/kg)+PRO (10 mg/kg) or NTX (10 mg/kg)+DPDPE (100 μg/kg)+PRO (10 mg/kg) for 4 weeks. Tumor shrinkage/growth was measured in animals daily and animal weights were measured every other day. Animals were euthanized when the tumor reached 5,000 mm³ and early euthanasia was performed if they displayed signs of distress or 10% loss of body weight. Three dimensions of tumor were measured using electronic calipers, and tumor volumes were calculated by the formula L×W²/2. The mean±SEM of tumor volume was calculated weekly for each experimental group, and the data are expressed as mean absolute tumor volume. After the animals were sacrificed, the subcutaneous tumors were excised intact from the rats to determine the tumor weight in grams and photographed to visualize differences in tumor morphology and compare size. A portion of the excised tumors was snap-frozen by immersion in liquid nitrogen and stored at −80° C. until further use.

### Survival Study:

The animals were treated with the same drug treatment regimens and euthanized as described previously. The behavior of tumor-bearing animals was monitored daily until the animals demonstrated an obvious health deterioration, or the maximum tumor volume allowed by ethical standards was reached; therefore, data are not truly absolute for animal survival. At the endpoint (prior to the onset of death), the animals were euthanized, and the survival curves were plotted in a Kaplan-Meier survival curve.

### Bioluminescent Imaging Study:

Tumor development or progression was monitored after injecting MDA-MB-231/Luc-GFP cells (GenTarget Inc., San Diego, CA) and followed the same drug treatment regimens for 6 weeks. Under isoflurane anesthesia, bioluminescent signals at the injection site (right flank) were detected by injecting i.p. with D-luciferin dissolved in 1×PBS at a concentration of 60 mg/kg (PerkinElmer Health Sciences, Inc., Shelton, CT) and transferring the animals to a light-tight chamber for optical imaging (in-Vivo FX PRO, Bruker Corp., Billerica, MA) using a charge-coupled device (CCD) camera (2048×2048 pixels) over a period of 5 minutes. Relative intensities of emitted light were represented as a pseudo color image ranging from purple (least intense) to white (most intense), generated in living image and superimposed onto the grayscale reference image. The signal intensity (SI) is expressed as average photon flux (photo/sec) from the tumor, with standard deviation.

### Collection of Tumor Samples for Histopathology:

At the end of the experiment, a portion of tumor tissues was fixed in 10% neutral buffered formalin overnight and embedded in paraffin, was cut into 5-μm-thick sections, and stained with H&E according to previous publications (8). Representative areas of tumor were analyzed for the presence of tumor cells and photographs were captured under a light microscope (20× magnification) as previously described.

### Immunoblotting and Immunohistochemistry Detection of Immune Cells:

For protein analyses, tumor tissues from in vivo experiments, enriched NK cells from the spleen, and NK-92 MI cancer cell co-incubated samples were lysed with a buffer containing protease and phosphatase inhibitors (25 mM Tris-HCl, pH 7.4; 150 mM NaCl; 1% Nonidet P-40; 1 mM EDTA; and 5% glycerol with Pierce Halt Protease Inhibitor). Protein (50 mg) from total lysate was loaded and separated on a 4-20% SDS-PAGE and transferred overnight to polyvinylidene difluoride membranes. Membranes were incubated with primary Ab for 12 hours at 4° C. in blocking buffer, 5% w/v nonfat dry milk in TBS, and 0.1% Tween 20. The following primary antibodies were purchased from Santa Cruz Biotechnology (Santa Cruz, CA): anti-perforin rabbit polyclonal (1:250), anti-granzyme B mouse monoclonal (1:200), anti-IFN-γ mouse monoclonal (1:250), anti-Snail (1:200), anti-Slug (1:200), and anti-Twist. Anti-E-cadherin (1:250), anti-N-cadherin (1:250), and anti-NKG2D goat polyclonal (1:500) were all from Abcam (Cambridge, MA). Membranes were washed and incubated with peroxidase-conjugated secondary Abs (1:5000; Vector Laboratories) for 1 hour at room temperature. Membranes were incubated with ECL western blot chemiluminescence reagent (Pierce) and exposed to x-ray film, which was developed, and densitometry was performed using Image J analysis software (National Institutes of Health). Each protein was normalized to corresponding intensities for β-actin.

Thin paraffin sections (5 μm) of tumors were stained using the ABC Elite Vectastain Kit (Vector Labs) according to the manufacturer's instructions using various primary antibodies. Details of all primary antibodies used are described above. In addition, cell proliferation-associated antigen (Ki-67; 1:200) were purchased from Abcam. After the primary antibody incubation and PBS wash, sections were incubated with peroxidase-coupled anti-rabbit Ig ImmPRESS reagent (Vector Laboratories, Inc.). Antigen localization was achieved by using the 3,3′-diaminobenzidine-peroxidase reaction, and sections were dehydrated, and cover slipped. To evaluate the immunohistochemical staining, sections were photographed using a Nikon-TE 2000 inverted microscope. Intensity of staining was categorized as negative and strongly positive (27). Brown-stained spots are immuno positive cells. Data are means±SEM obtained from 3 serial sections of 5 to 6 animals in each group.

### Isolation of PBMC for NK Cell Activity and Flow Cytometry Analysis:

Peripheral blood mononuclear cells (PBMC) from control and drug-treated rats were isolated from freshly obtained EDTA whole blood by density gradient purification (Lymphoprep™, Stemcell Technologies™, Vancouver, BC, Canada) using 15 mL SepMate™ tubes (Stemcell Technologies™) as per the manufacture's protocol. The pelleted cells (PBMC) were counted in a hemocytometer and assayed for viability by the trypan blue dye exclusion method. PBMC cell pellets from 3 animals in each group were pooled, washed three times with PBS, and finally resuspended in 2 ml RPMI medium containing 10% heat-inactivated FBS (HI-FBS) and used for NK cell cytotoxicity. Aliquots of PBMC were frozen down at −80° C. by resuspending cells in freezing media containing HI-FBS and 10% DMSO in aliquots of 5-10×10⁶ cells/mL using Nalgene Mr. Frosty Cryo 1° C. freezing containers (Thermo Fisher Scientific) before storage in liquid nitrogen. For flow cytometric analysis, frozen PBMC was thawed and resuspended in RPMI-1640 and was evaluated for viability by trypan blue, and all were 295% viable, then washed with PBS before starting flow cytometer experiments.

Flow Cytometry Analysis of Immune Cells from Tumor Tissue, PBMC, and Splenocytes:

Tumor-infiltrating immune cells were collected from MDA-MB-231 cell xenograft tumors to examine and compare the immune cell population among control and drug-treated animals. Briefly, single-cell suspensions of tumor tissue were achieved by mincing the tissue with a scalpel blade using a crisscross method, ground with 1-ml of ACK lysing buffer to remove red blood cells. The cell suspensions were filtered through a 70-micron sieve and then washed with PBS. Cells from the tumor were collected and dispersed with 100 μl of PBS and stained by the addition of fluorescence-conjugated antibodies. For flow cytometry experiments, single-cell suspension derived from tumors, PBMC, and splenocytes were used to characterize the immune cell population. Fluorophore-conjugated antibodies were used to detect surface markers of specific cell types and markers are as follows: for NK cells (CD161a (10/78)), tissue macrophage (RT1B (OX-6)), dendritic cells (CD103 (OX-62)), peripheral monocytes (CD11b (ED8)), B cells (CD45RA (OX-33)), and DAPI (Sigma) was used to exclude dead cells. A list of antibodies, dye, isotype controls and beads used for the flow-cytometry experiment was given in Tables 2 and 3.

Immune cells from the tumor, PBMC, and splenocyte samples were examined by following the gating strategy as described below. First, side scatter area (SSC-A) versus forward scatter area (FSC-A) were used to exclude cellular debris, then forward scatter height (FSC-H) versus FSC-A were used to select single cells. Single cells were sub-gated using DAPI viability dye and a further live cell population was used to select markers. Expression of a particular cell surface marker in a specific population of cells was identified and represented as single parameter histograms. Unstained cells and compensation beads (BD Biosciences, CA) were used to set voltages and create single stain negative and positive controls. Additional negative controls (isotype control and fluorescence minus one (FMO)) were also used to determine any non-specific binding in the present study. The data collected from each sample were exported and analyzed using FlowJo™ version 10.7.

TABLE 2
List of antibodies & dyes used in flow cytometry

| Cell type | Reactivity | Conjugate | Clone | Company | Catalogue # | Dilution |
|---|---|---|---|---|---|---|
| Monocytes | Mouse anti rat | FITC | ED8 | Bio-Rad, | MCA619F | 1:100 |
| | CD11b | | | Philadelphia, | | |
| | | | | PA | | |
| B-cells | Mouse anti-rat | APC-Cy ™7 | OX-33 | BD | 561624 | 1:100 |
| | CD45RA | | | Biosciences, | | |
| | | | | San Jose, CA | | |
| NK cells | Mouse anti-rat | PE | 10/78 | BD | 555009 | 1:100 |
| | CD161a | | | Biosciences, | | |
| | | | | San Jose, CA | | |
| Macrophage | Mouse anti-rat | PerCP | OX-6 | BD | 557016 | 1:100 |
| | RT1B | | | Biosciences, | | |
| | | | | San Jose, CA | | |
| Live/Dead | | DAPI | | Sigma, St. | D-9542 | 1 μg/mL |
| cells | | | | Louis, MO | | |

TABLE 3
List of isotype control & beads used in flow cytometry

| Reactivity | Conjugate | Clone | Company | Catalogue # | Dilution |
|---|---|---|---|---|---|
| Mouse IgG1 | FITC | | Bio-Rad, | MCA1209F | 1:100 |
| Negative Control | | | Philadelphia, PA | | |
| Mouse IgG1, κ | APC-Cy ™7 | MOPC-21 | BD Biosciences, | 557873 | 1:100 |
| Isotype Control | | | San Jose, CA | | |
| Mouse IgG1, κ | PE | MOPC-31C | BD Biosciences, | 550617 | 1:100 |
| Isotype Control | | | San Jose, CA | | |
| Mouse IgG1, κ | PerCP | MOPC-31C | BD Biosciences, | 550672 | 1:100 |
| Isotype Control | | | San Jose, CA | | |

| | | |
|---|---|---|
| Anti-Rat Ig, κ/Negative Control Compensation | BD Biosciences, | 552844 |
| Particles Set | San Jose, CA | |

Enrichment of NK Cells from Spleen and PBMC:

After the end of the drug treatment period, the spleen tissues from control and drug-treated rats were processed, and RBCs and granulocytes were removed from splenocyte suspensions by density centrifugation using Histopaque 1083 (Sigma-Aldrich) as previously described (8). Splenocytes (˜10×10⁷ cells per spleen) were extracted from the middle layer, washed with RPMI-1640 (Gibco), and resuspended in buffer (PBS, 0.5% BSA). Splenocytes were incubated with primary Abs conjugated to FITC (BD Biosciences), anti-CD6 (OX52; T cells), anti-CD45RA (OX-33; mature B cells), and anti-RT1B (OX-6; dendritic cells and macrophages), followed by secondary incubation with anti-FITC microbeads, according to the manufacturer's instructions (Miltenyi Biotec). NK cells were then enriched by magnetic separation (negative selection) using an AutoMACS Magnetic Separator (Miltenyi Biotec). The enriched fraction consistently yielded ˜5×10⁶ cells per spleen with a purity of ˜80-90%. Enriched NK cells were used for cytotoxicity assays or lysis with appropriate buffer for protein analyses. Peripheral blood mononuclear cells (PBMC) from rats were isolated from freshly obtained heparinized whole blood using the SepMate 15 ml tube (Stemcell Technologies, Vancouver, BC, Canada) according to the manufacturer's instructions. The pelleted cells (PBMC) were counted in a hemocytometer and assayed for viability by trypan blue exclusion. PBMC cell pellets from 3 animals in each group were pooled, washed three times with PBS, and finally resuspended in 2 ml RPMI medium containing 10% FBS and used for NK cell cytotoxicity.

### NK Cell Cytotoxicity:

The cytotoxicity of enriched NK cells from PBMC and the spleen against NK-sensitive YAC-1 (ATCC) target cells was determined by calcein AM assays as previously described (8). YAC-1 (murine lymphoma) cells were grown and maintained in RPMI 1640 without phenol red (Gibco), containing 1% penicillin-streptomycin (Gibco) and 10% FBS (Gibco). YAC-1 cells were washed and incubated with 5 mM calcein AM (Sigma-Aldrich) in serum-free RPMI-1640 for 10 minutes at 37° C. Labeled YAC-1 cells were washed and plated into U-bottom 96-well plates (Falcon) at a concentration of 5×104 cells per well. NK cells were added at a E:T (20:1) ratio in triplicates. YAC-1 cells in RPMI alone were to determine spontaneous calcein AM release, whereas maximal release was achieved by lysing target cells in buffer (0.1% Triton X-100). Enriched NK cells from PBMC and the spleen were preincubated for 12 hours with IL-2 at 37° C. (100 ng/ml; R&D Systems) prior to 4-hour incubation with YAC-1 target cells. All assays were analyzed and percent cytotoxicity for each sample was calculated as previously described (8).

### Cytokines Multiplex Immunoassay:

After the end of the drug treatment period, plasma samples from control and drug-treated rats were processed and used for cytokine detection. Cytokines were quantified in plasma samples using the ProcartaPlex multiplex immunoassay technology. These immunoassays are bead-based assays for protein quantification based on the principles of a sandwich Elisa with the use of Luminex® xMAP® (multianalyte profiling) technology. The Rat Cytokine & Chemokine 22-plex ProcartaPlex Panel is a preconfigured multiplex immunoassay kit that measures 22 protein targets using Luminex xMAP technology. Plasma samples were diluted with universal assay buffer (1:1) and added into a 96-well flat bottom plate to the magnetic beads. The plate was sealed and covered before incubation of 2 hours at room temperature with 500 rpm shaking. After inserting the plate into the handheld magnetic plate washer, the plate was washed 3 times using a specific wash buffer. 25 μL of detection antibody mixture was to the plate and incubated it for 30 minutes at room temperature at 500 rpm. The plate was washed 3 times. Then 50 μL of SAPE solution were added to the plate before a novel 30-minute incubation at room temperature at 500 rpm. The plate was washed 3 times. Finally, 120 μL of reading buffer were add to the plate and incubated for 5 minutes at room temperature at 500 rpm before reading and analyzing with the Luminex™ instrument (MAGPIX® xPONENT®, R&D System, ThermoFisher). Median fluorescence index (MFI) was calculated and presented.

### Apoptosis Analysis

The effects on apoptotic regulatory protein levels and signaling pathway were determined by treating MDA-MB-231, MDA-MB-468, and T47D cells with a 100-μM concentration of the drugs alone or in combination or vehicle alone for 48 hours. After the treatment period, cell were extracted and used for Western blot analysis of anti-apoptotic p-Bcl-xL and p-Bcl2, effector proteins such as p-Bax, pro-apoptotic molecule p-Bim, effector caspase 3 and Cytochrome c levels. Representative blots and mean densitometric values are presented as ratio of β-actin in the histograms. Data presented are mean±SEM (n=5-6 samples/group) and were analyzed using one-way ANOVA with the Newman-Keuls multiple comparisons post hoc test.

### Statistical Analysis

Statistical analysis was performed using GraphPad Prism 5. The significance between treatment and controls was assessed using one-way ANOVA between treatments and two-way ANOVA between treatments and days of exposures. For survival data, Kaplan-Meier curves were generated using Graph Pad Prism. P<0.05 was considered significant.

The effects of NTX, a MOR antagonist and DOR activator (13, 14); DPDPE, a DOR agonist (14); and PRO, a non-selective B2AR antagonist, alone or in combination, on growth and proliferation of three human breast cancer cells: MDA-MB-231, MDA-MB-468, and T47D are demonstrated in the following examples.

### Example 1

### Verification that Breast Cancer Cells Contain Beta-Adrenergic and Opioidergic Receptors

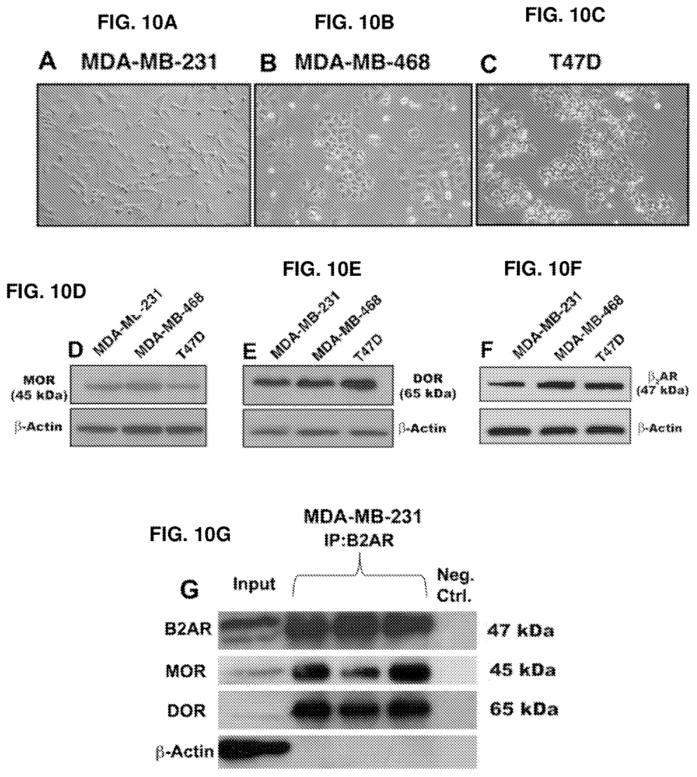

Using STR profile authenticated cell lines, it was first confirmed that these human breast cancer cells maintained their morphological phenotype (FIGS. 10A-C) and expressed primary marker receptors (ER, PR, and HER2; data not shown) during passages as described elsewhere (20). It was verified that these three cell lines expressed MOR (FIG. 10D), DOR (FIG. 10E), and B2AR receptors (FIG. 10F) by immunoblotting. It was also confirmed that opioid receptors (MOR and DOR) and B2AR receptors can physically interact in MDA-MB-231 cells (the cell line used for in vivo studies described below). These cell lysates were immunoprecipitated with B2AR antibody and probed with MOR and DOR antibodies, which showed a detectable amount of MOR (45 kDa) and DOR (65 kDa) bands in addition to a B2AR band of 47 kDa (FIG. 10G).

### Example 2

### Effects of Beta-Adrenergic and Opioidergic Drugs Alone or in Combination on Cancer Cell Viability

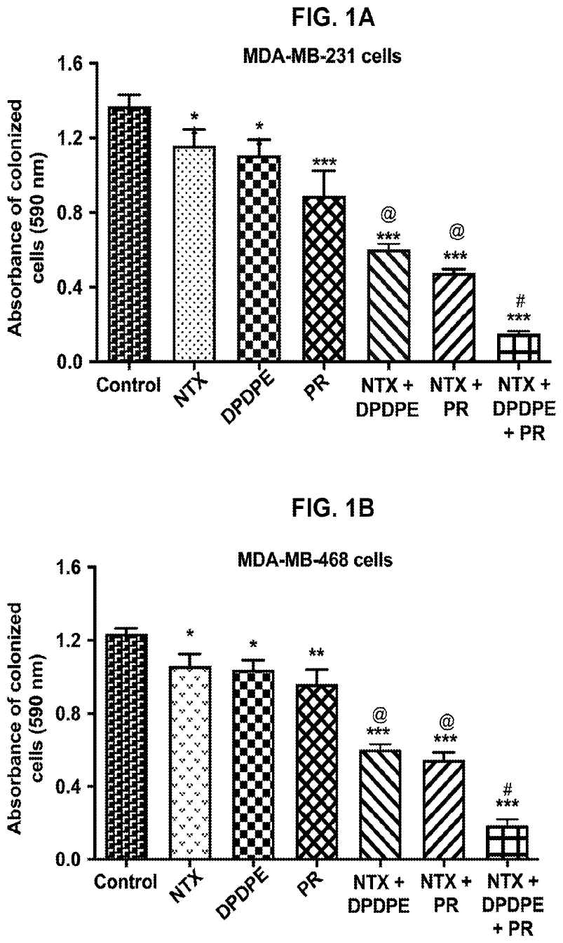

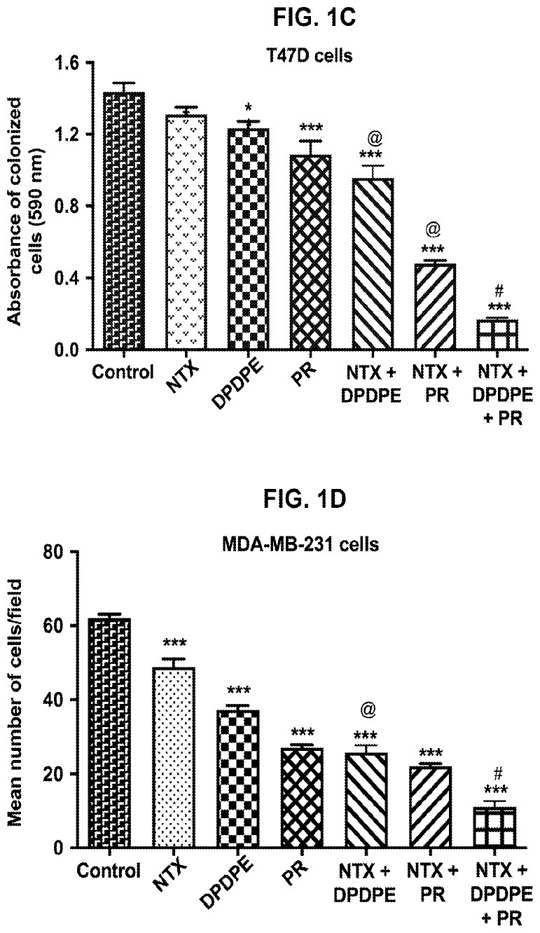

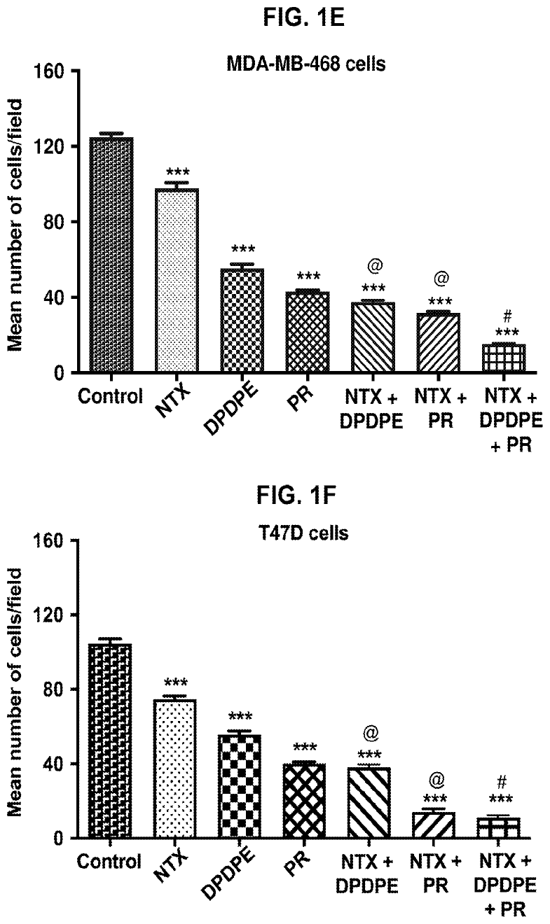

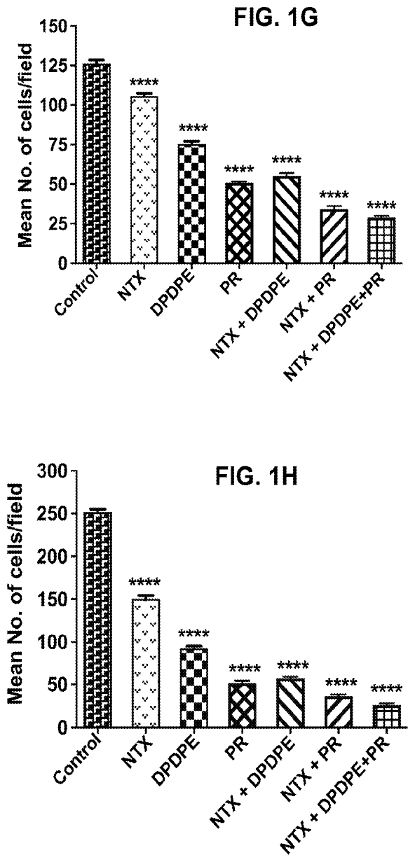

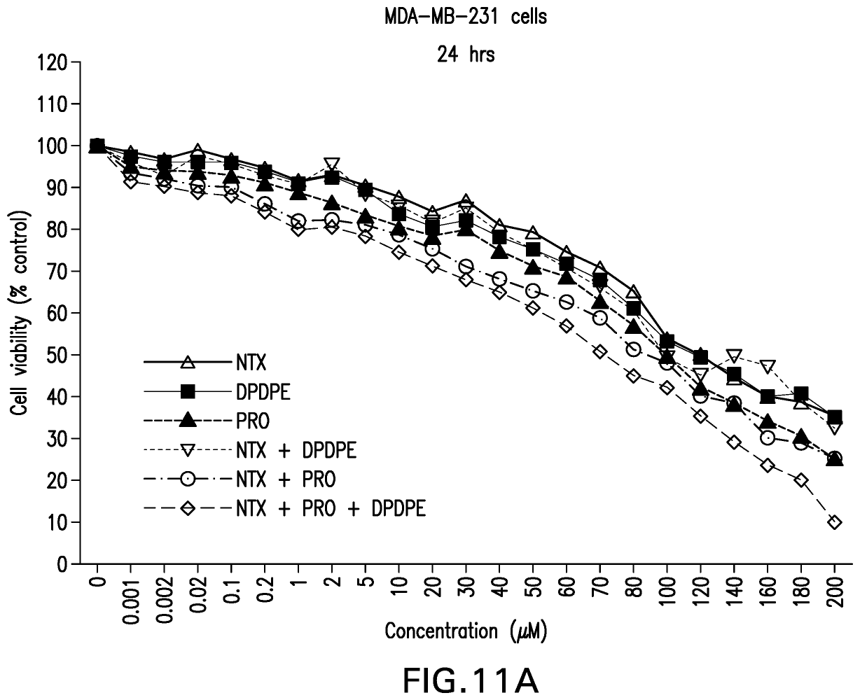

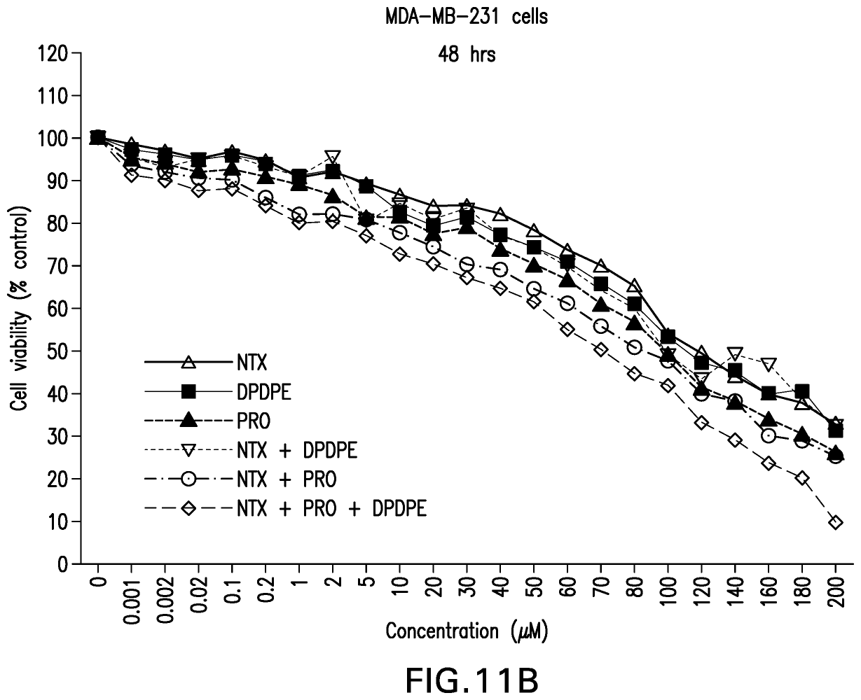

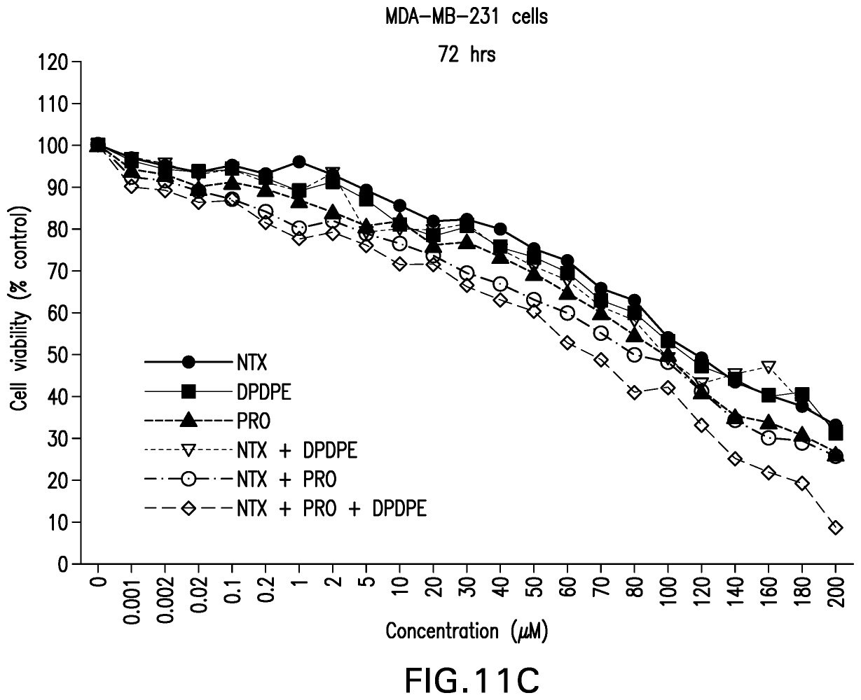

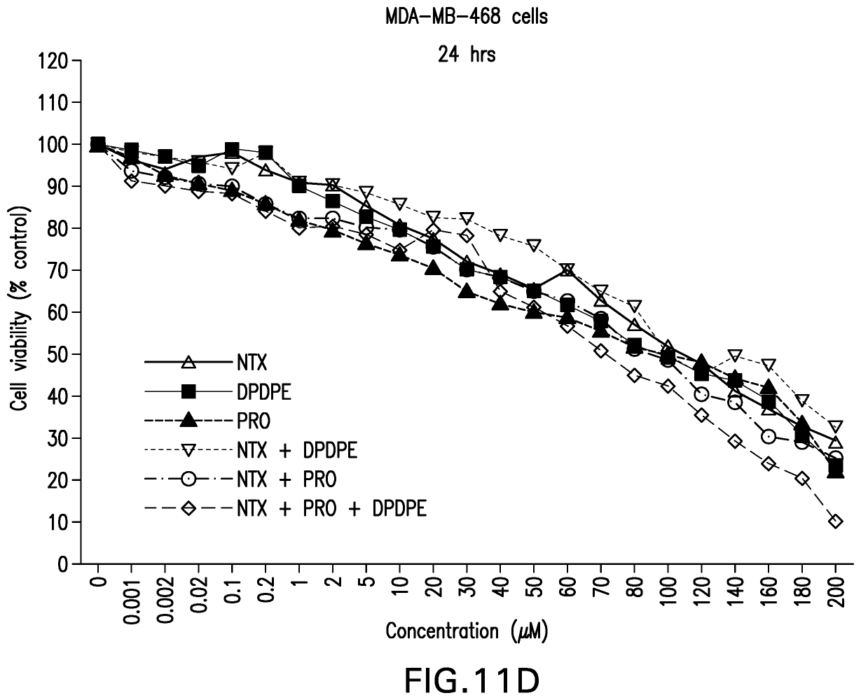

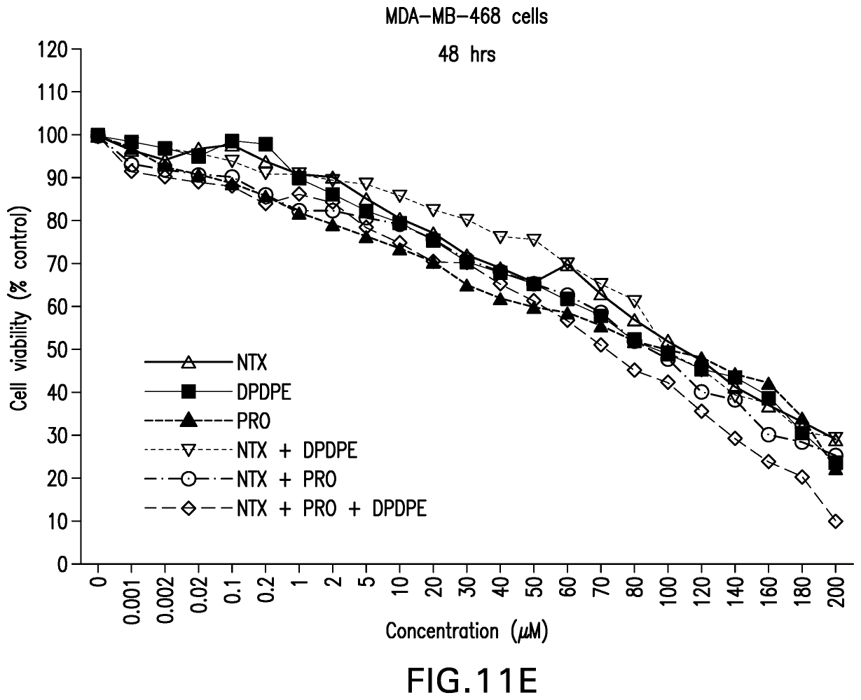

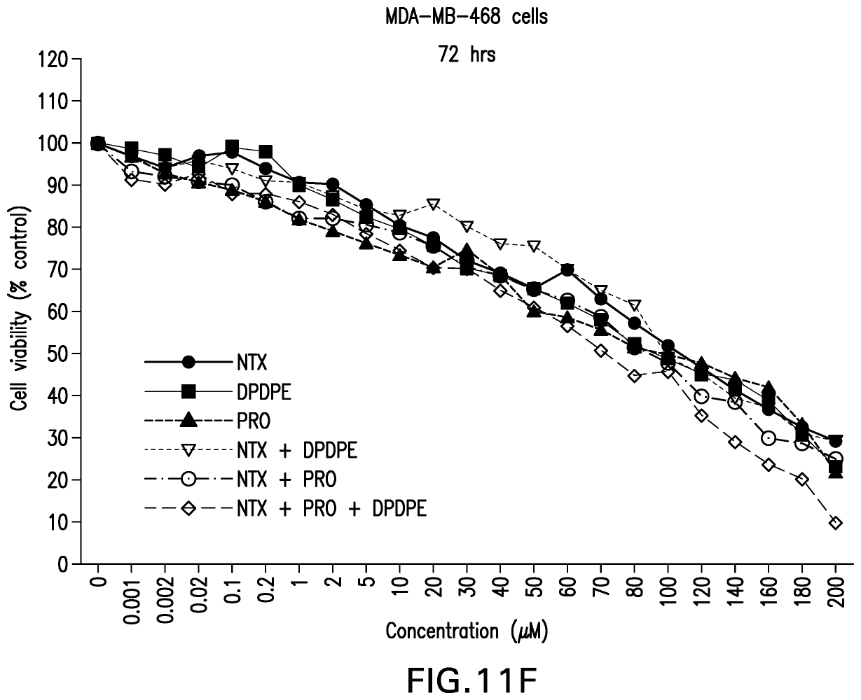

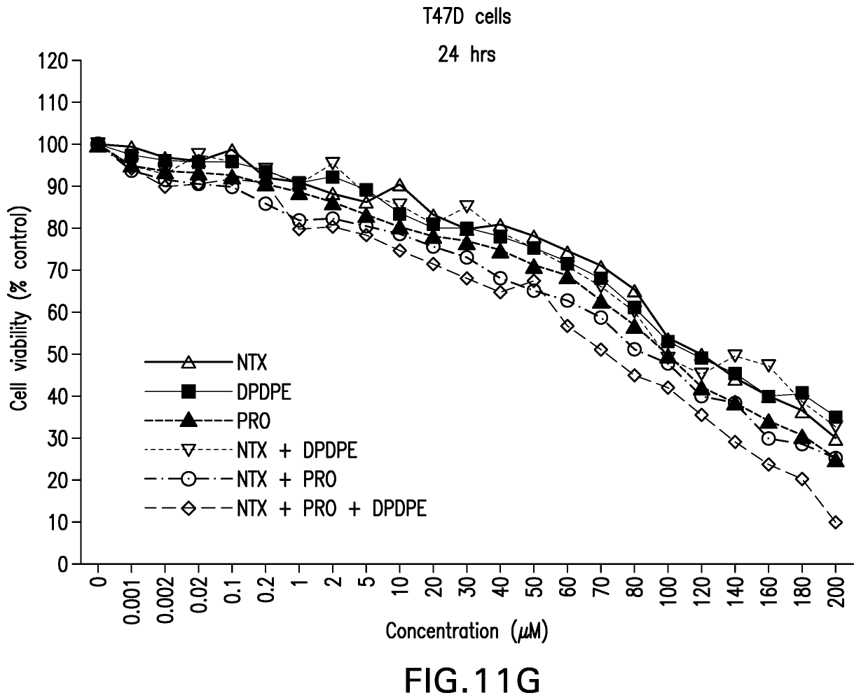

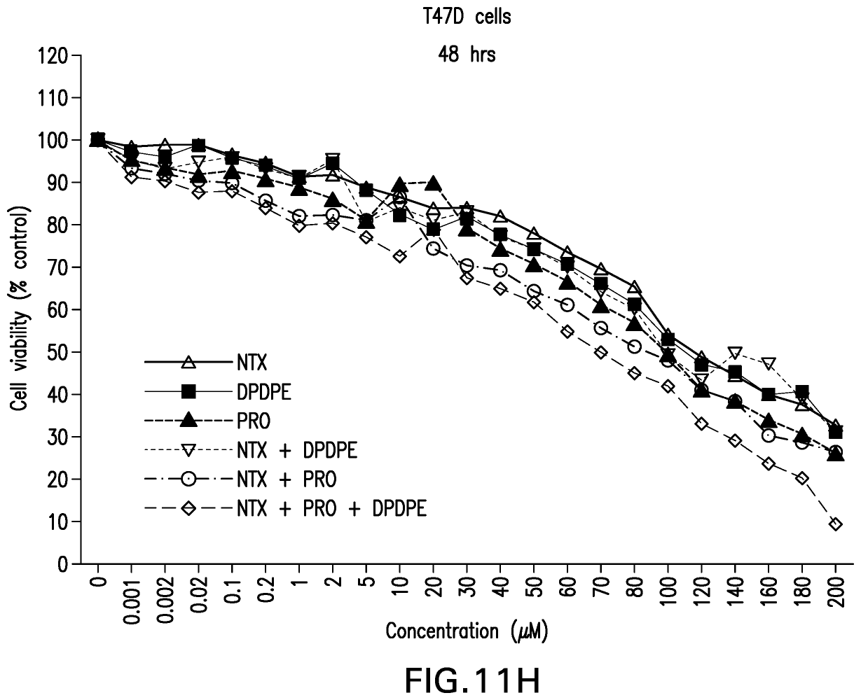

Direct effect of opioidergic and adrenergic agents on breast cancer cell viability was tested following treatment with various concentrations (0.001 μM to 200 PM) of NTX, DPDPE, and PRO alone or in combination on MDA-MB-231, MDA-MB-468, and T47D cells for 24, 48, and 72 hours. These drugs, either individually or in combination, caused a dose-dependent inhibition of cell viability at all-time points (FIGS. 11A-1). The effects of these drugs at different exposure times or on different cell lines yielded similar growth suppression. The combination treatments of drugs were more effective than single treatments. The IC₅₀ dose varied between different treatment groups and time of treatment (NTX, 140-120 PM; DPDPE, 140-115 μM; PRO, 100-120 μM: NTX+DPDPE, 100-120 μM; PRO+NTX, 90-100 μM; PRO+NTX+DPDPE, 70-75 μM; FIG. 1A). The combination of the three drugs, NTX+DPDPE+PRO, displayed the most potent growth inhibition with the IC₅₀ of ˜ 70-75 μM in all cell lines and treatment times.

Table 4 below provides a summary of inhibitory concentration 50% (IC50) values at 24, 48, and 72 h for all three cell lines after naltrexone (NTX), DPDPE, propranolol (PRO), NTX+DPDE, NTX+PRO, and NTX+DPDPE+PRO treatment. Values were obtained from three independent experiments in quadruplicate wells of each treatment and expressed as ±§ SEM (standard error of the means). IC50 values between groups were analyzed using Two-way ANOVA followed by Tukey's multiple comparison.

TABLE 4
Cell Viability

| | | IC₅₀ (μM) ± SEM^{§} | | |
|---|---|---|---|---|
| | | 24 h | 48 h | 72 h |
| MDA- | NTX | 140.79 ± 0.15 | 120.49 ± 0.38 | 120.84 ± 1.65 |
| MB-231 | | (n = 12) | (n = 12) | (n = 12) |
| | DPDPE | 140.43 ± 0.39 | 120.83 ± 0.36 | 119.94 ± 1.07 |
| | | (n = 12) | (n = 12) | (n = 12) |
| | PR | 100.50 ± 0.38 | 100.52 ± 0.29 | 100.19 ± 0.97 |
| | | (n = 12) | (n = 12) | (n = 12) |
| | NTX + DPDE | 120.50 ± 0.38 | 105.02 ± 0.58 | 104.60 ± 1.30 |
| | | (n = 12) | (n = 12) | (n = 12) |
| | NTX + PR | 90.16 ± 0.51 | 100.86 ± 0.52 | 100.89 ± 1.65 |
| | | (n = 12) | (n = 12) | (n = 12) |
| | NTX + DPDPE + | 75.45 ± 0.40 | 70.47 ± 0.30 | 70.79 ± 0.92 |
| | PR | (n = 12) | (n = 12) | (n = 12) |
| MDA- | NTX | 122.43 ± 0.63 | 120.82 ± 1.82 | 120.17 ± 1.23 |
| MB-468 | | (n = 12) | (n = 12) | (n = 12) |
| | DPDPE | 115.10 ± 1.23 | 118.49 ± 1.22 | 120.11 ± 2.19 |
| | | (n = 12) | (n = 12) | (n = 12) |
| | PR | 120.50 ± 0.86 | 120.52 ± 0.98 | 100.36 ± 1.04 |
| | | (n = 12) | (n = 12) | (n = 12) |
| | NTX + DPDE | 115.15 ± 1.24 | 115.19 ± 0.96 | 100.85 ± 0.70 |
| | | (n = 12) | (n = 12) | (n = 12) |
| | NTX + PR | 100.49 ± 0.39 | 100.53 ± 1.44 | 90.56 ± 1.11 |
| | | (n = 12) | (n = 12) | (n = 12) |
| | NTX + DPDPE + | 74.92 ± 1.26 | 72.14 ± 1.71 | 69.96 ± 1.20 |
| | PR | (n = 12) | (n = 12) | (n = 12) |
| T47D | NTX | 125.43 ± 1.37 | 122.16 ± 1.72 | 120.17 ± 1.61 |
| | | (n = 12) | (n = 12) | (n = 12) |
| | DPDPE | 125.10 ± 1.21 | 120.16 ± 1.17 | 120.46 ± 0.04 |
| | | (n = 12) | (n = 12) | (n = 12) |
| | PR | 100.16 ± 0.74 | 105.02 ± 1.69 | 100.69 ± 0.45 |
| | | (n = 12) | (n = 12) | (n = 12) |
| | NTX + DPDE | 105.10 ± 2.06 | 100.69 ± 0.78 | 100.85 ± 1.81 |
| | | (n = 12) | (n = 12) | (n = 12) |
| | NTX + PR | 90.16 ± 0.50 | 95.86 ± 1.36 | 90.06 ± 0.67 |
| | | (n = 12) | (n = 12) | (n = 12) |
| | NTX + DPDPE + | 74.95 ± 1.22 | 72.14 ± 0.97 | 70.12 ± 1.04 |
| | PR | (n = 12) | (n = 12) | (n = 12) |

### Example 3

### Effects of a Single Drug or Drug Combination on Cancer Cell Clonogenicity, Migration, and Invasion

The effects of NTX, DPDPE, and PRO on cancer cell function were determined by evaluating their effects on colony formation, migration, and invasion (21, 22). Because the IC₅₀ dose of these agents ranged between 70-140 μM in the cell growth inhibition curve, a 100 μM dose was used for all beta-adrenergic and opioidergic agents for these assays. The effects of these drugs on colony formation are shown in FIGS. 1A-C and 12A-C. In MDA-MB-231 cells, NTX, DPDPE, and PRO alone showed moderate inhibition; NTX in combination with DPDPE or PRO showed more inhibition than alone; and NTX in combination with PRO and DPDPE showed the maximal inhibition of colony formation. The effect of the three-drug combination produced a significant more effect than any other drug combination. Similar effectiveness of these drugs was observed on MDA-MB-468 and T47D cells.

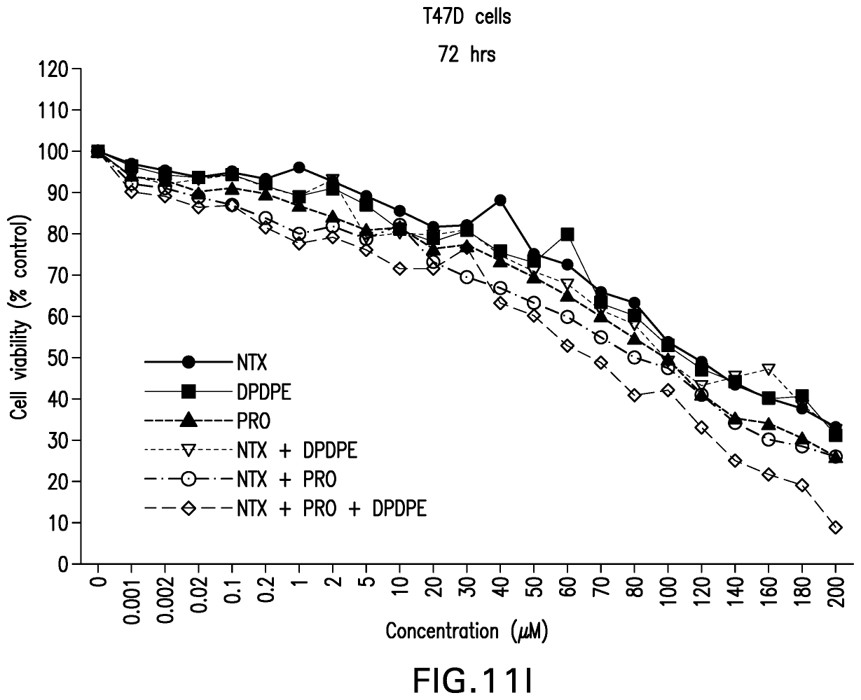

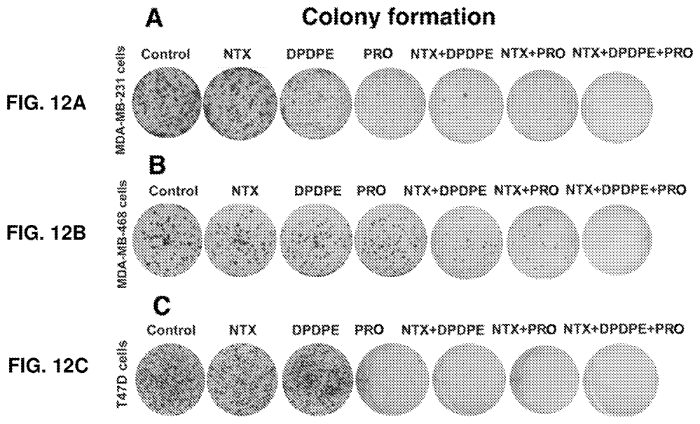

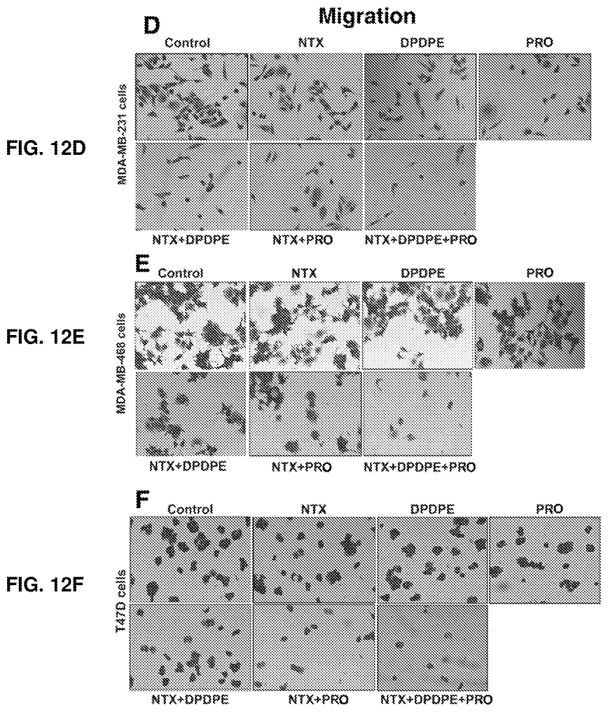

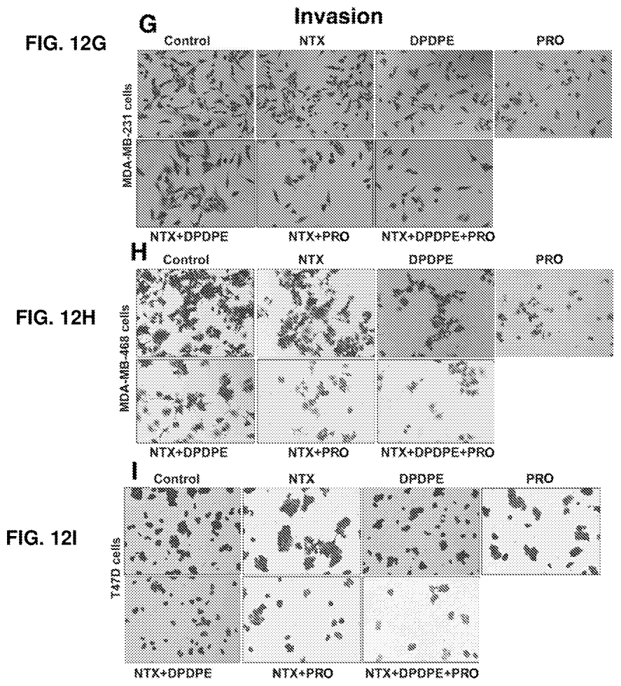

The effects of the 100 μM dose of beta-adrenergic and opioidergic agents on MDA-MB-231, MDA-MB-468, and T47D cell mobility were tested using transwell cell migration assay. The results of migration assay are shown (FIGS. 1D-F and 12D-F). These data indicate that single drugs, NTX, DPDPE, or PRO alone, produced significant but moderate inhibitory effects on MDA-MB-231 (FIGS. 1D and 12D), MDA-MB-468 (FIG. 1E and 12E), and T47D cells (FIGS. 1F and 12F). A combination of NTX+DPDPE, NTX+PRO, or NTX+DPDPE+PRO showed medium to high inhibition of cell migration, respectively, in these three cell lines.

Next, the effectiveness of a 100 μM dose of beta-adrenergic and opioidergic agents on cell invasion of MDA-MB-231 (FIGS. 1G and 12G), MDA-MB-468 (FIGS. 1H and 12H), and T47D cells (FIGS. 11 and 12I) was tested on a Matrigel™ coated membrane. The results show that NTX, DPDPE, and PRO decreased cell invasion alone and in combination. The combination of NTX+DPDPE+PRO produced the maximum inhibition of cell invasion in these three cell lines.

### Example 4

### Effects on Cell Cycle Changes and Apoptotic Regulatory Protein Levels in Cancer Cells

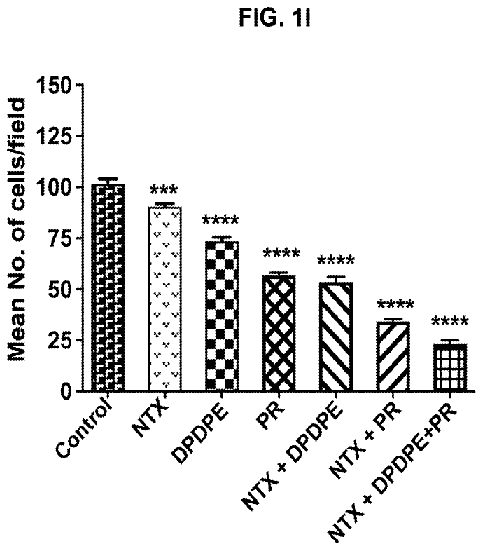

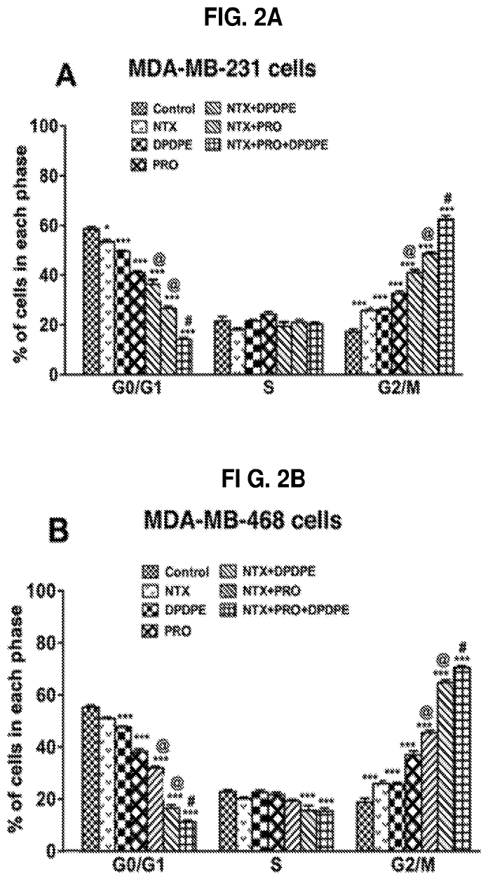

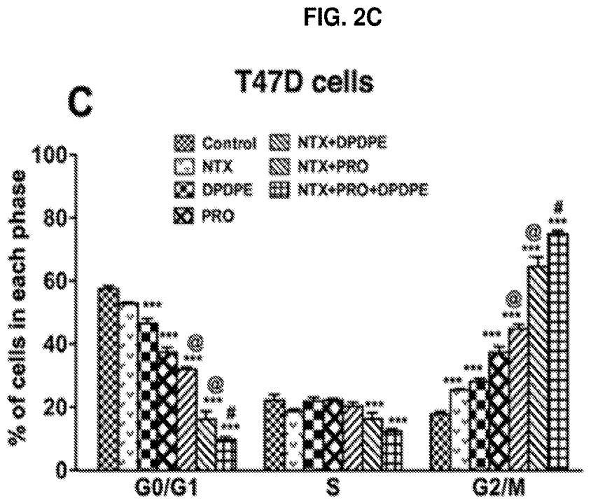

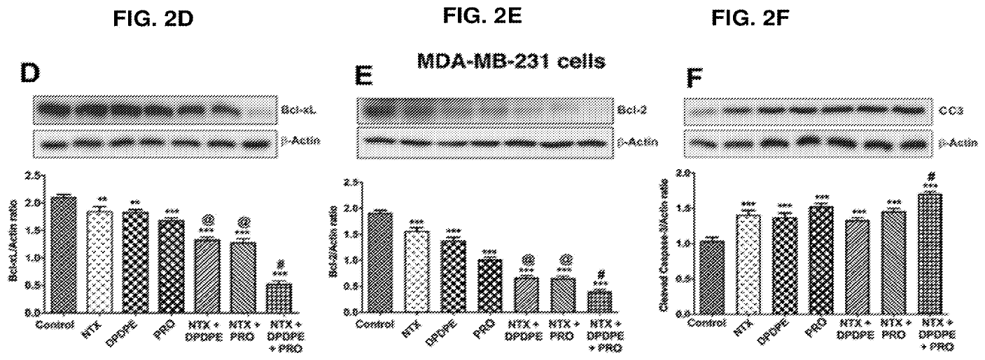

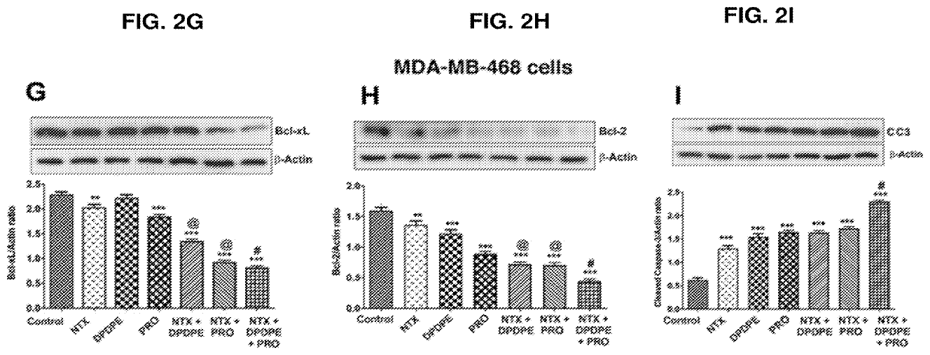

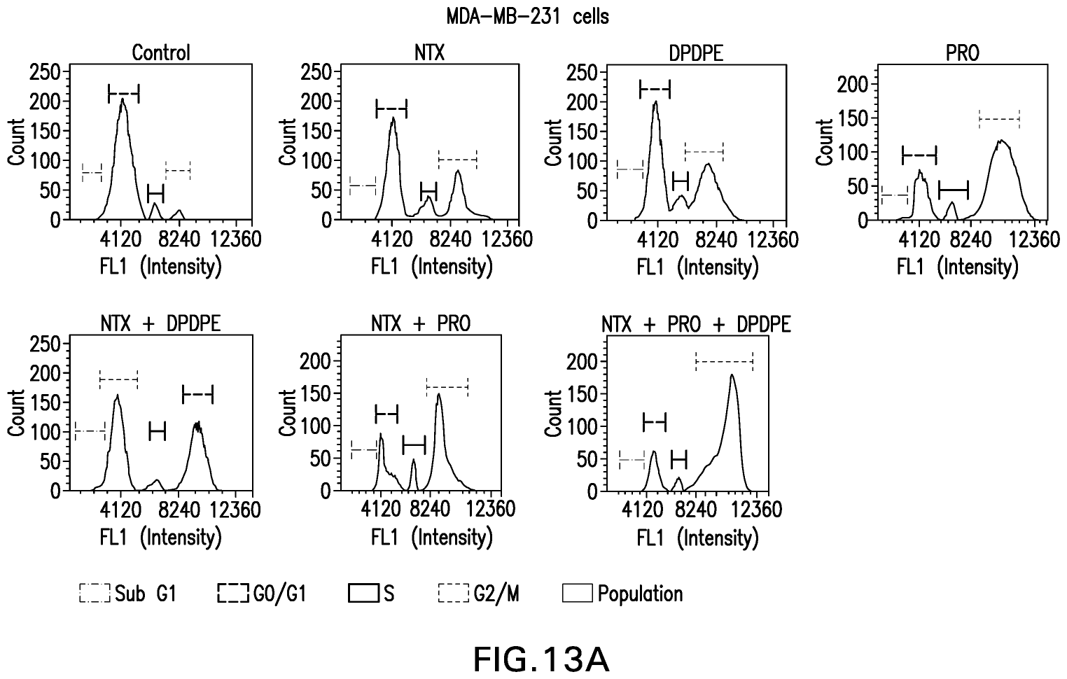

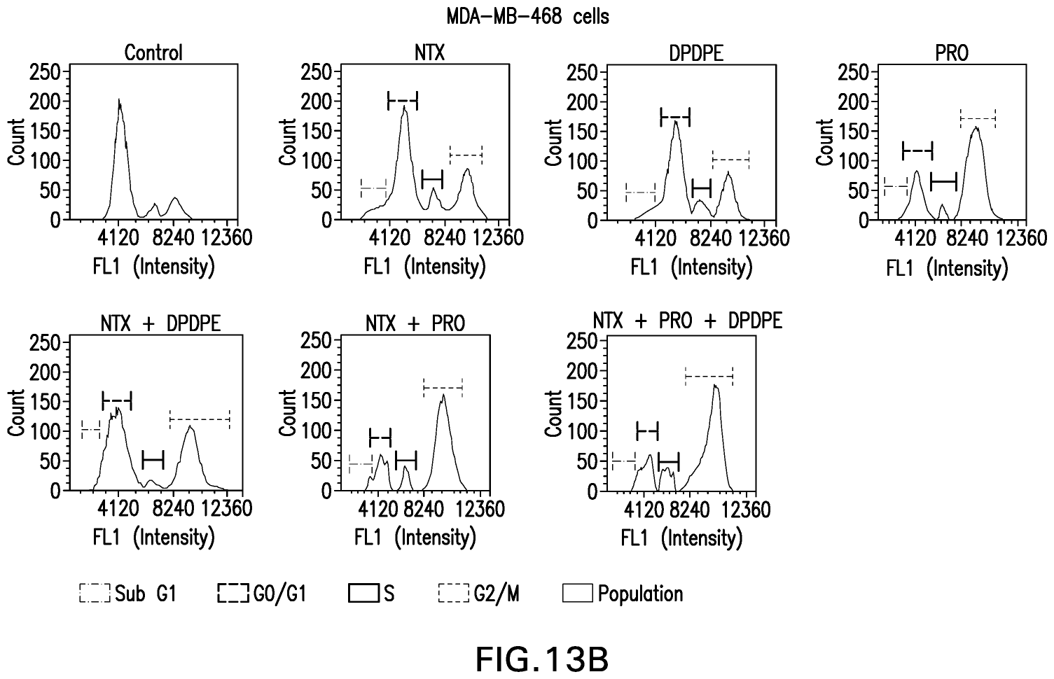

Since the beta-adrenergic and opioidergic agents decreased the breast cancer cell viability, it was determined if the effect was associated with the changes in cell cycle arrest (23). The cell cycle distribution of MDA-MB-231, MDA-MB-468, and T47D cells with or without the drug treatment was measured for 24 hours, and the data are shown in FIGS. 2A-C and FIGS. 13A-C. Treatment with 100 μM NTX, DPDPE, or PRO alone reduced the number of MDA-2 MB-231 cells in G0/G1 phase, produced no significant changes in the number in S phase, but increased the number of these cells in G2/M phase (FIG. 2A and FIG. 13A). The effects of these drugs on cell cycle distributions were enhanced when they were combined doubly or triply than when they were given singly. The largest effect on G2/M shift was observed following treatment with NTX+DPDPE+PRO. The effects of these drugs on cell cycle distributions of MDA-MB-468 and T47D cells were similar with some small differences; unlike in MDA-2 MB-231 cells, NTX treatment alone moderately but not significantly reduced the number of these cells in the G0/G1 cycle, and NTX+PRO and NTX+DPDPE+PRO treatments reduced the cell numbers in S phase in MDA-MB-468 and T47D cells (FIGS. 2B-C and FIGS. 13B-C).

Because these beta-adrenergic and opioidergic drugs promote breast cancer cells to undergo cell cycle arrest in G2/M, the possibility arises that these cells may be undergoing cellular death (23). A western blot analysis was done to determine anti-apoptotic BCL-2 family isoforms Bcl-xL and Bcl-2 (24) and proapoptotic cleaved caspase 3 (CC3; 25) levels in MDA-MB-231 (FIGS. 2D-F), MDA-MB-468 (FIGS. 2G-1), and T47D cells (FIGS. 2J-L) after treatment with a single drug (NTX, DPDPE or PRO) or a drug combination (NTX+DPDPE, NTX+PRO or NTX+DPDPE+PRO) for 48 hours at 100 μM concentration. It was found that the tested beta-adrenergic and opioidergic drugs decreased the levels of Bcl-xL and Bcl-2 while they increased the levels of CC-3 in all cell lines. The effects of these drugs on apoptotic regulatory proteins were enhanced when they were combined doubly or triply than when they were given singly. The NTX+DPDPE+PRO combination, in general, produced the maximal effects.

### Example 5

### Effects on Expanded Human NK Cell Cytolytic Activity

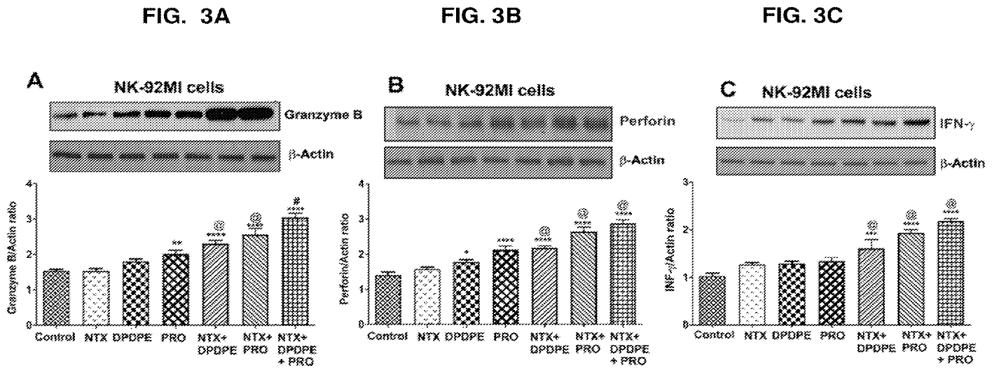

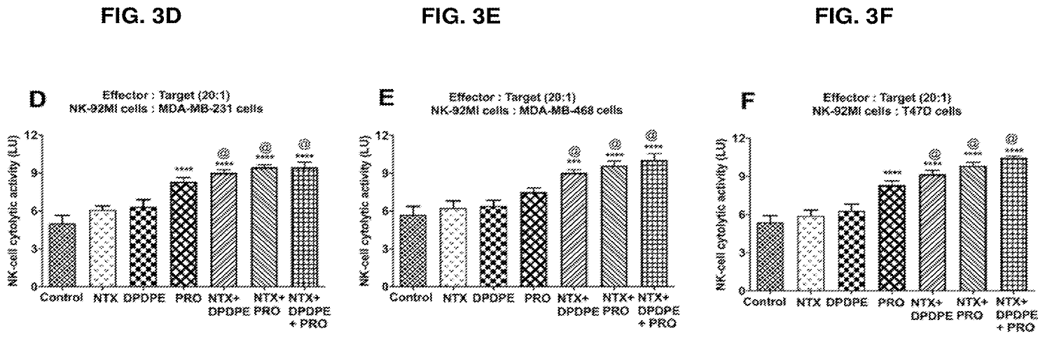

Tests were conducted to determine whether the levels of cytotoxic protein and the cytolytic activity of human NK cells (NK-92 MI) are responsive to the treatments with beta-adrenergic and/or opioidergic agents. It was found that NK cells positively responded to these drugs, particularly when they were combined (FIGS. 3A-C). In these cells, granzyme B levels (FIG. 3A) and perforin (FIG. 3B) levels were moderately elevated by PRO while markedly elevated by NTX+DPDPE, NTX+PRO, and NTX+DPDPE+PRO. IFN-γ levels were markedly elevated by NTX+DPDPE, NTX+PRO, and NTX+DPDPE+PRO (FIG. 3C). The cytolytic results of NK cells are shown in FIGS. 3D, 3E, and 3F for MDA-MB-231, MDA-MB-468, and T47D cells, respectively). The cytolytic activity of NK cells were significantly elevated on the breast cancer cells treated with combination drugs as compared to a single drug or no drug (control) treatment.

### Example 6

### Effect on the Growth of Cell Line Derived Tumor Xenograft

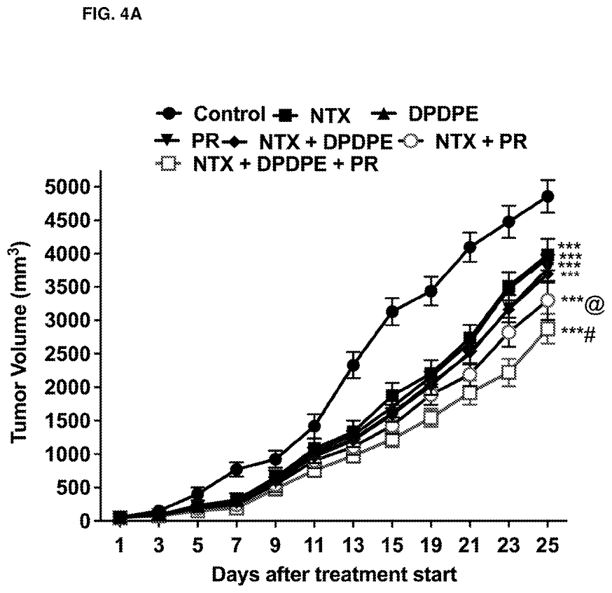

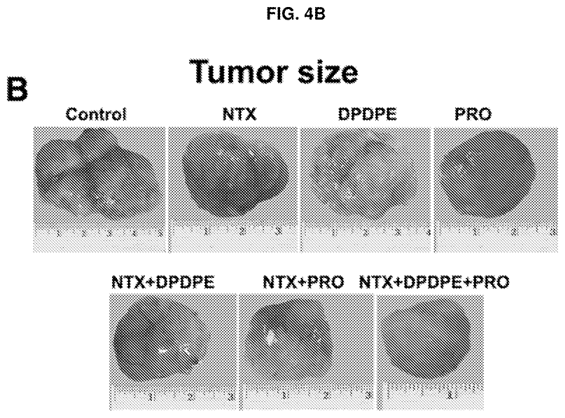

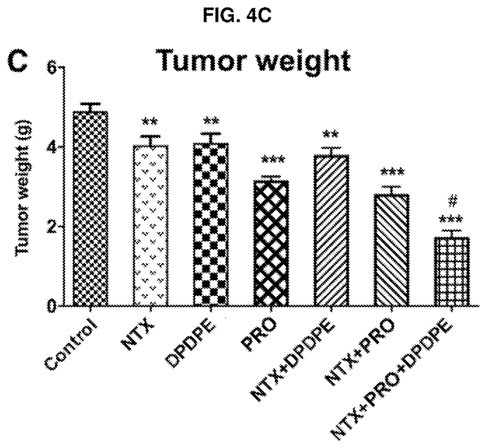

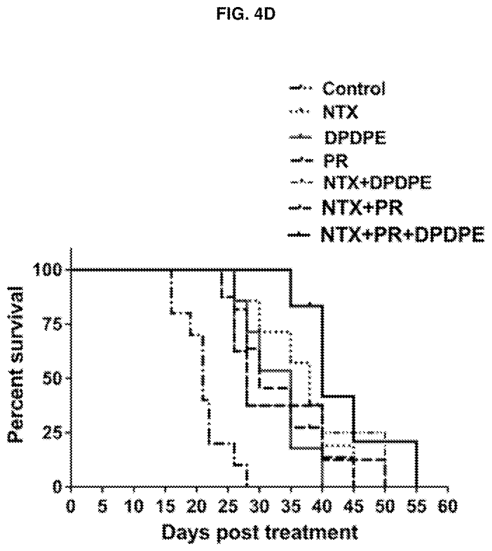

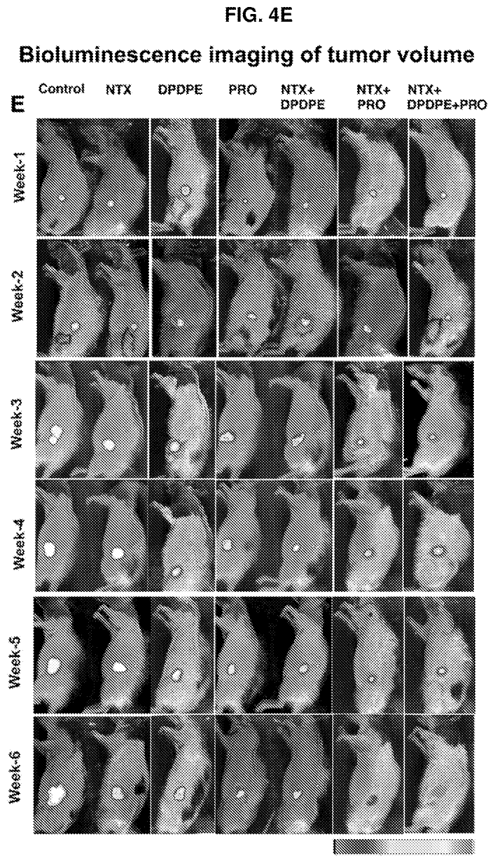

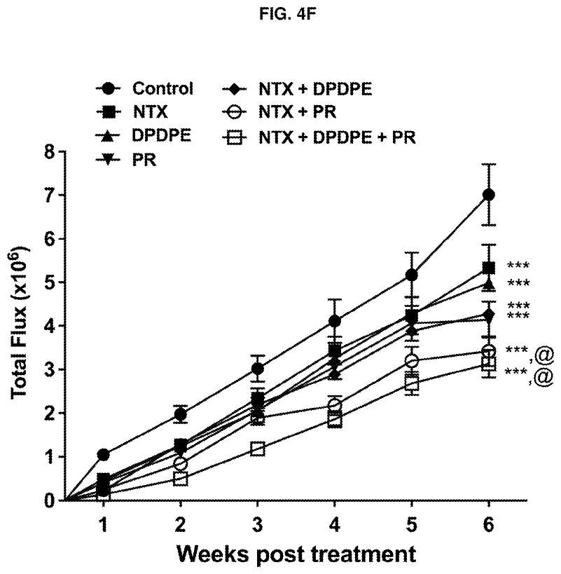

To investigate the in vivo effects of beta-adrenergic and opioidergic agents alone or in combination on breast cancer growth, MDA-MB-231 cells and T-cell-deficient athymic nude rats were used, where the predominant immune cells are NK cells (26). In vitro studies using three different breast cancer cell lines showed similar growth inhibitory effects of beta-adrenergic and opioidergic agents, and therefore, the in vivo studies were limited to using only MDA-MB-231 cells. After tumor xenotransplantation, when tumors reached palpable size (˜50 mm³), the animals were treated with a single drug or combination of drugs or saline (control) for a period of about 4 weeks. The xenograft tumor grew well and reached a volume of approximately 5000 mm³ within a few weeks in control animals (FIG. 4A). Tumor growth as determined by tumor volume was reduced by the treatment with all the drugs tested. The drug effect on tumor growth was more when drugs were combined than when given alone, and the maximum growth inhibitory effect, about 50% of control, was achieved by the triple-drug combination group (NTX+DPDPE+PRO).

At termination, xenograft tumors were excised from each of the seven treatment groups to verify the effect of drugs on tumor shrinkage/growth. The representative photographs of a tumor from each treatment group revealed that the tumors were homogenous and dense, surrounded by less areas of bleeding, and lower in tumor size in drug-treated animals than in the control animals (FIG. 4B). Visual inspections revealed that drug combination treatments, especially treatments with three-drug combinations, markedly reduced the vascularization of the tumor and the tumor size. Data of tumor wet weight also show that these agents were effective in reducing tumor weight, and the drug effect on tumor weight was more potent when combined, particularly the combination of NTX+DPDPE+PRO (FIG. 4C).

In another set of animals, the same drug treatment regimen was used for survival analysis using the Kaplan-Meier method (30). The results of the survival experiment show that the life span of control animals was about 28 days, and the animals treated with a single drug, NTX, PRO, or DPDPE, had mean life spans of 45, 45, and 40 days, respectively, which is significant when compared to 28 days in control animals (FIG. 4D). Interestingly, the combination of NTX+DPDPE, NTX+PRO, and NTX+PRO+DPDPE improved the survival of animals to 50, 50, and 55 days (mean values), respectively,

To monitor the effect of drugs on tumor shrinkage/growth in real time, MDA-MB 231/Luc-GFP cells in a subcutaneous flank tumor model for bioluminescence imaging (28) in nude rats was used. It was observed that bioluminescent intensity of the tumor generally showed an increasing trend like that of the tumor volume (FIGS. 4A and 4E-F). The combination therapy (NTX+DPDPE or NTX+PRO) resulted in the decrease of both luminescent signals and bioluminescent intensity over the 6-week period, and the effect was more pronounced in the triple combination of NTX+DPDPE+PRO treatment.

### Example 7

### Effect on Cell Mitosis, Cell Proliferation, Cellular Apoptosis, and Epithelial-Mesenchymal Transition in Tumor Xenograft

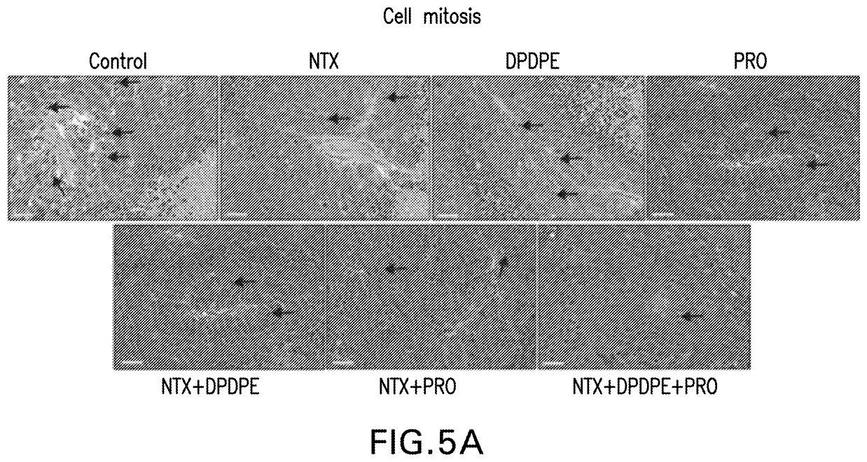

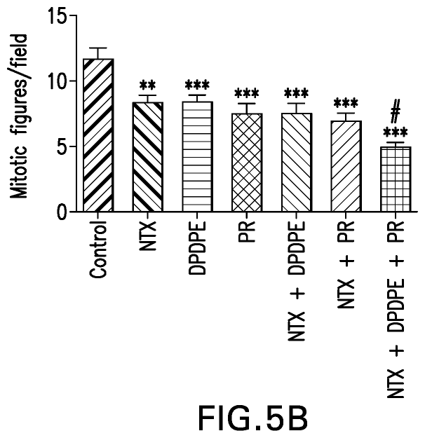

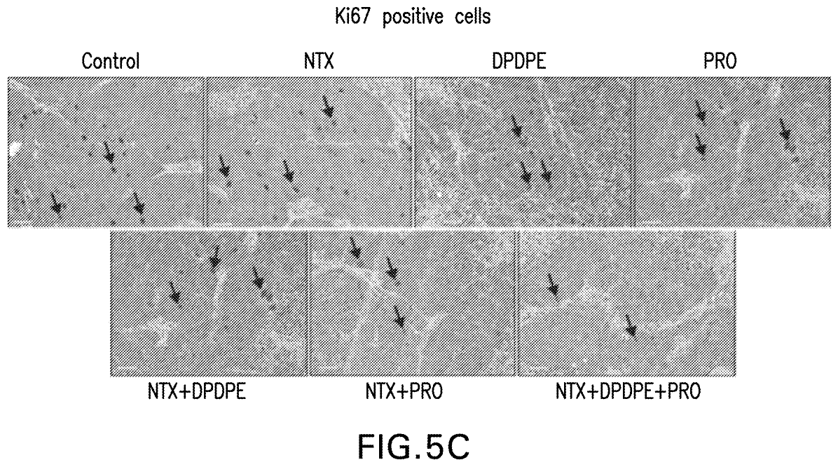

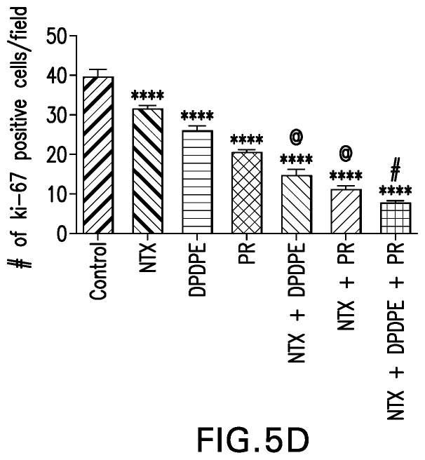

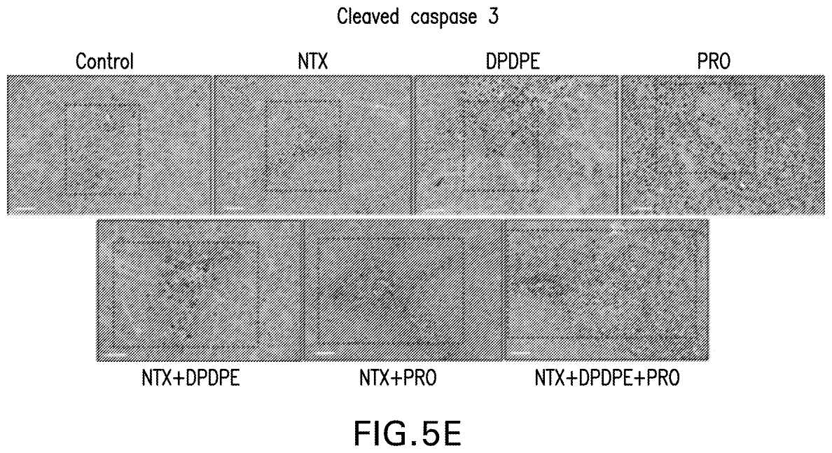

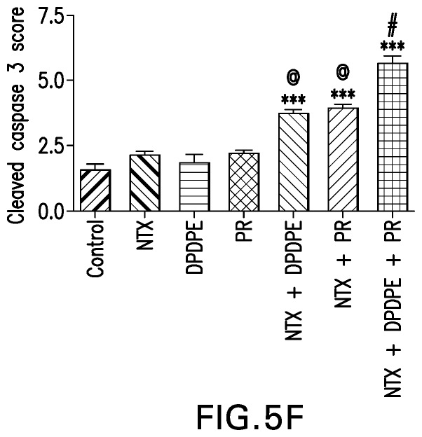

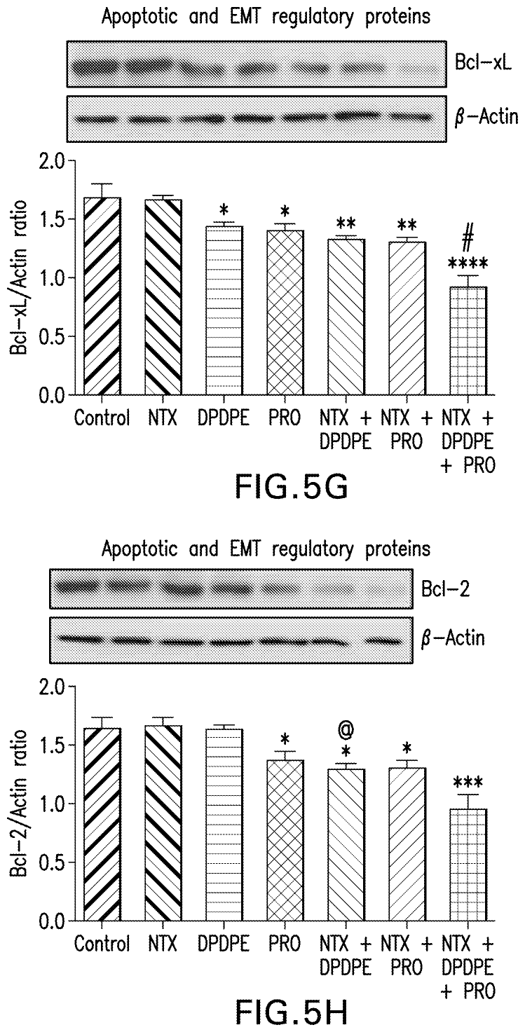

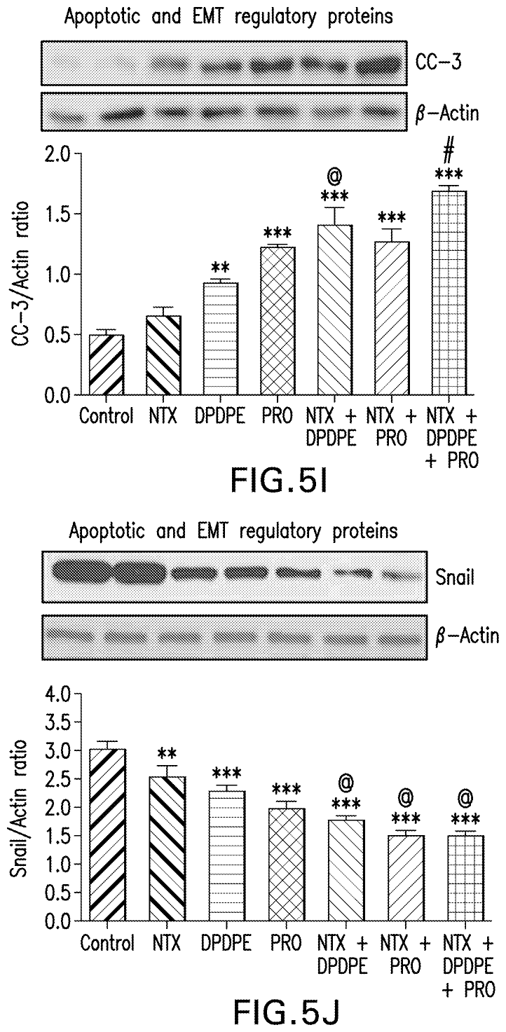

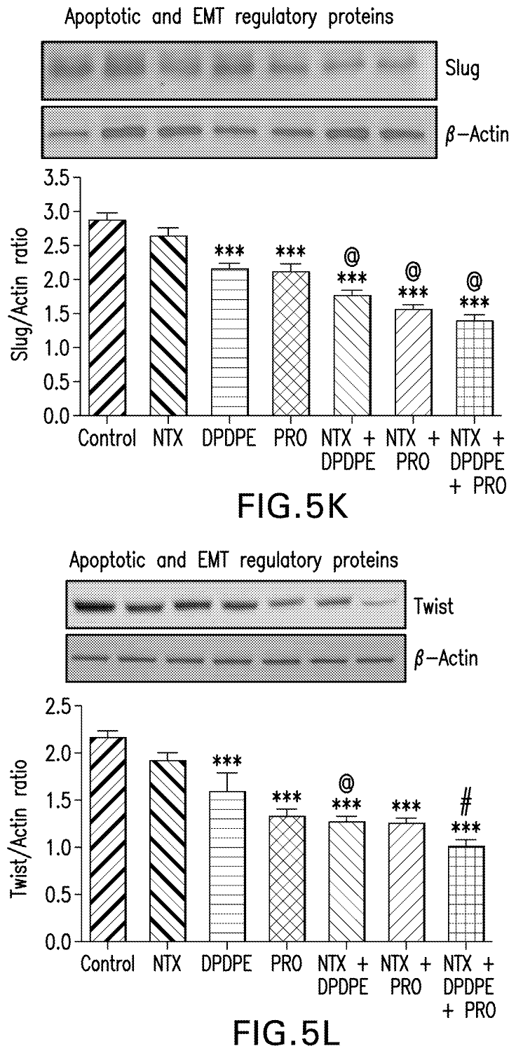

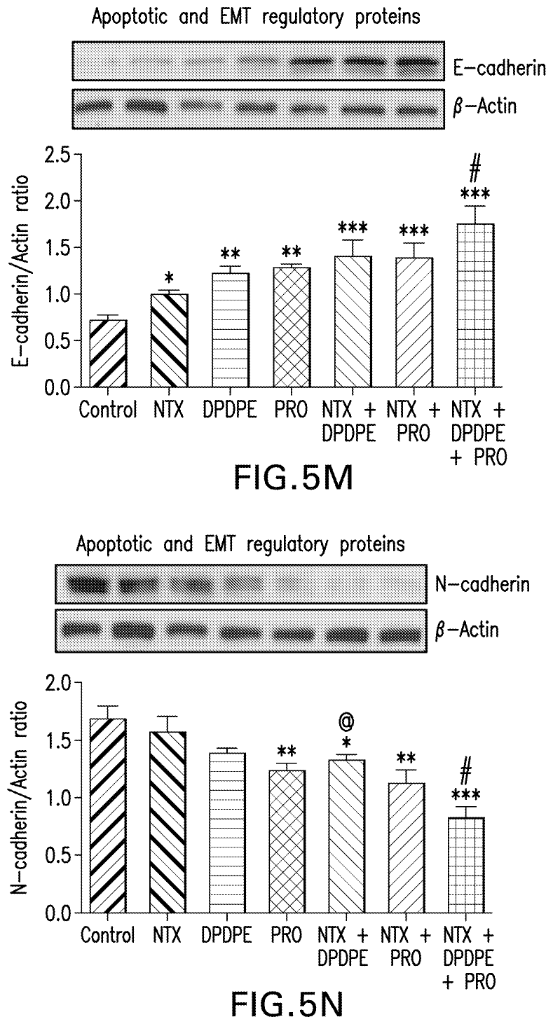

Histological examination of xenograft tumors from each group showed that control animals had healthy intact tumor cells (clear borders) with the presence of many mitotic figures (dividing cells shown by arrows) in the tumor (FIGS. 5A-B). Xenograft tumors from beta-adrenergic and opioidergic treated animals, especially with the combination therapy of NTX+DPDPE or NTX+PRO, had a decrease in the number of mitotic figures. The triple combination of NTX+DPDPE+PRO treatment showed more prominent reduction in mitotic figures (seen very rarely). An immunostaining procedure was used to evaluate the number of ki-67 positive cells as a marker for cell proliferation (29) and CC3 as an indicator of apoptotic bodies (30) in each group. Data show that control animals had a higher number of proliferating cells (FIG. 5C, arrows, and FIG. 5D), but they had a lower number of apoptotic cells in the center of the tumor (FIG. 5E, boxes with broken lines, and FIG. 5F). Single-drug treatment moderately decreased the ki-67 levels in the tumor while combined-drug treatment, particularly triple-drug treatments, markedly decreased the ki-67 levels in the tumor. Conversely, combined-drug treatments, particularly triple-drug treatments, markedly increased the CC3 levels in the tumor.

A growing body of research shows that Bcl-xL and Bcl-2 act as pro-survival factors for the resistance to apoptosis and survival of cancer cells (24). Whereas caspases, especially activated (cleaved) forms of caspases such as CC3, play a crucial role for initiating and executing apoptosis within a cell (25). Western blot data of Bcl-xL, Bcl-2, and CC3 levels in tumor tissue are shown in FIGS. 5G-1. Data show the treatment with NTX, DPDPE, or PRO alone or in combinations decreased the levels of Bcl-xL and Bcl-2 while they increased the level of CC3. The most potent effects of these drugs on the levels of these proteins in the tumor were observed when triple drugs were combined.

The levels of epithelial-mesenchymal transition (EMT) factors (Snail, Slug, and Twist), epithelial (E-cadherin) markers, and mesenchymal (N-cadherin) markers in tumor tissues were measured since these proteins are known to play important roles in acquiring the invasive and metastatic property of cancer cells (31). Immunoblotting results of EMT factors and markers indicated that single-drug (PRO or DPDPE) treatment reduced the levels of Snail (FIG. 5J), Slug (FIG. 5K), and Twist (FIG. 5L), with a concomitant decrease in the level of N-cadherin (FIG. 5N) and an increase in the level of E-cadherin (FIG. 5M) in tumors as compared with those in control tumors. Notably, this trend of modulation in these proteins was highly significant and showed an additive effect in the tumor of animals treated with combination drugs NTX+DPDPE or NTX+PRO, especially NTX+DPDPE+PRO.

### Example 8

### Effects of Drugs on Immune Cell Functions in the Tumor Xenograft Host

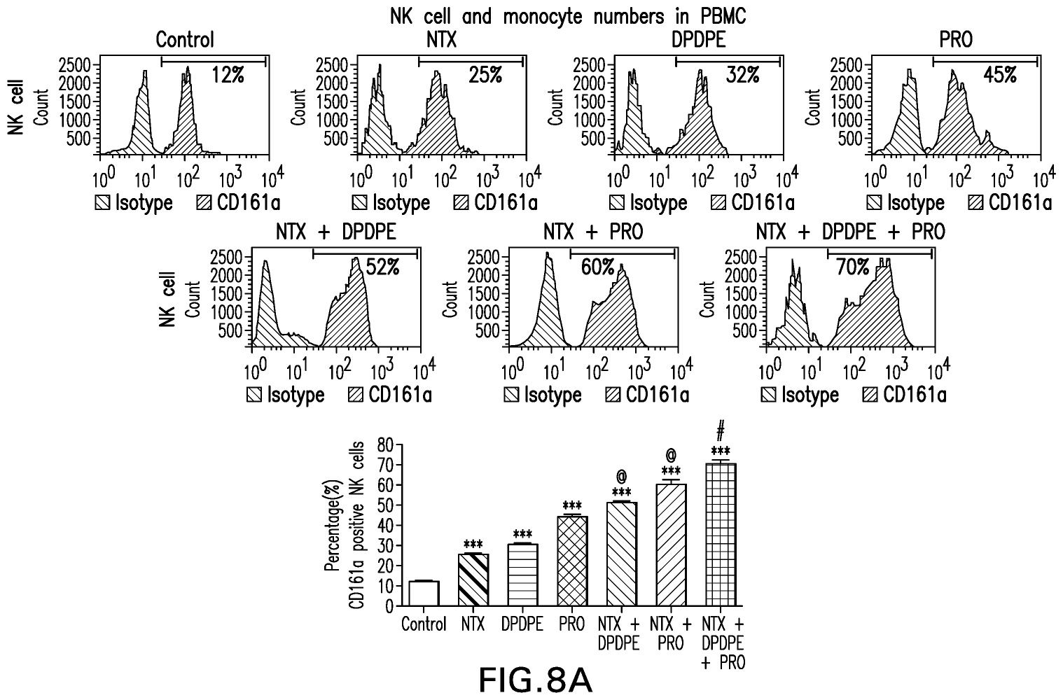

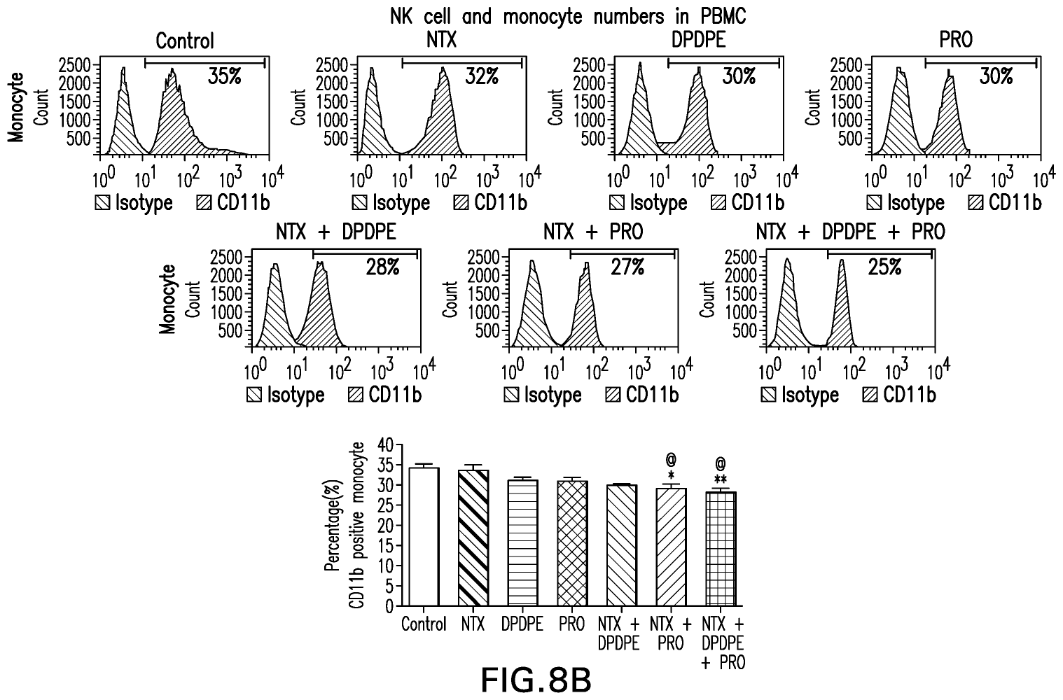

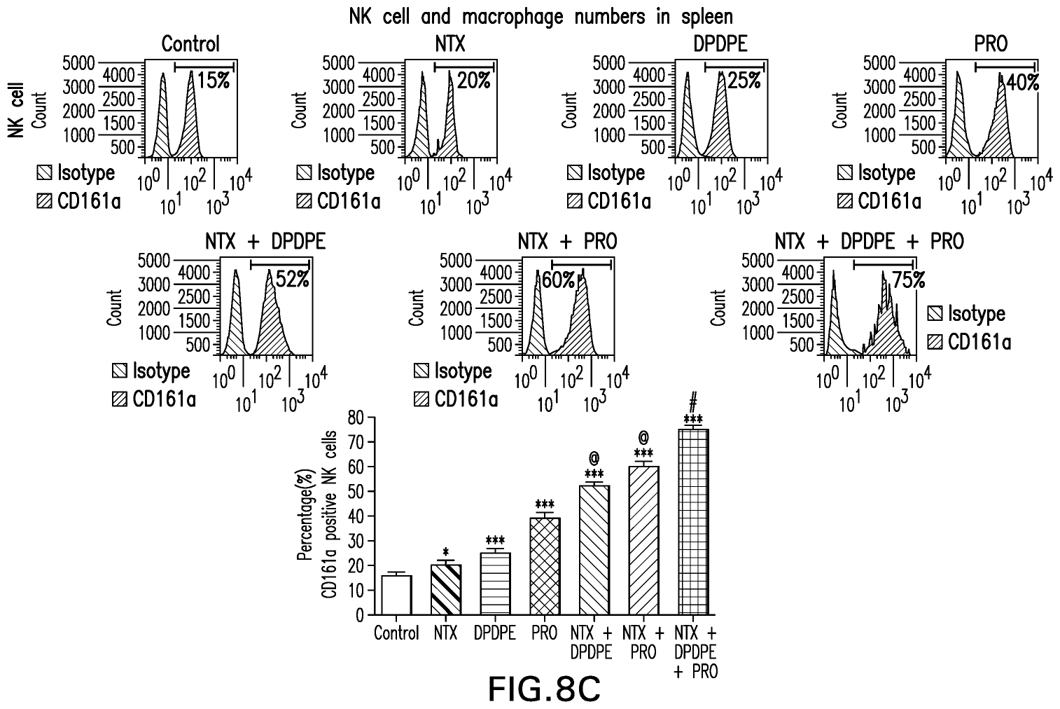

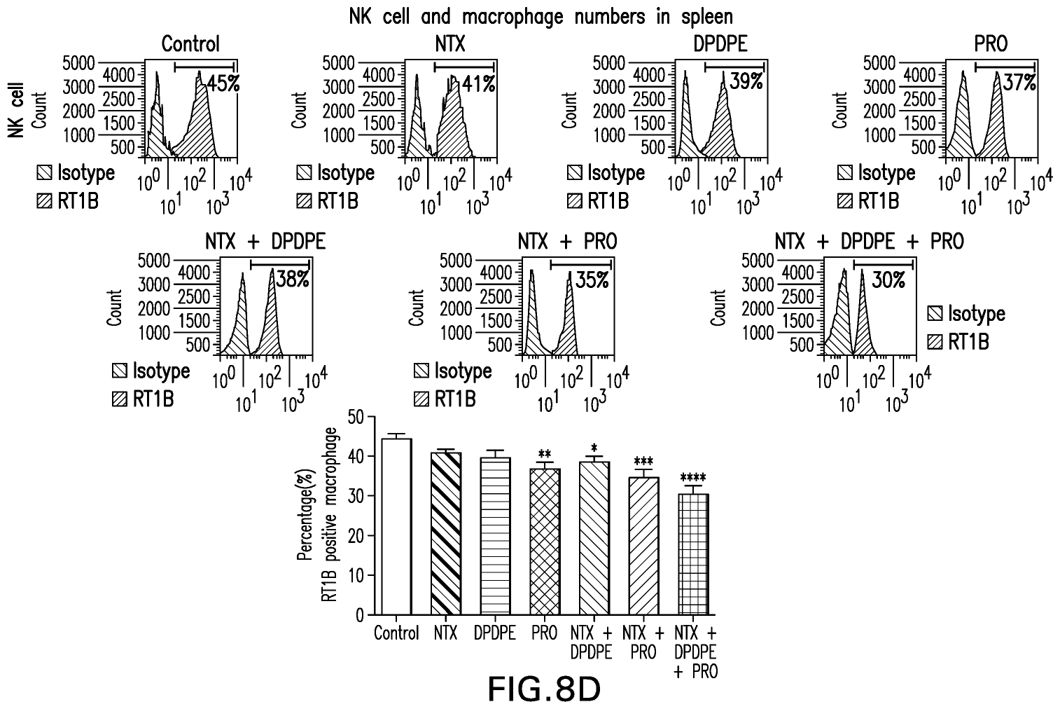

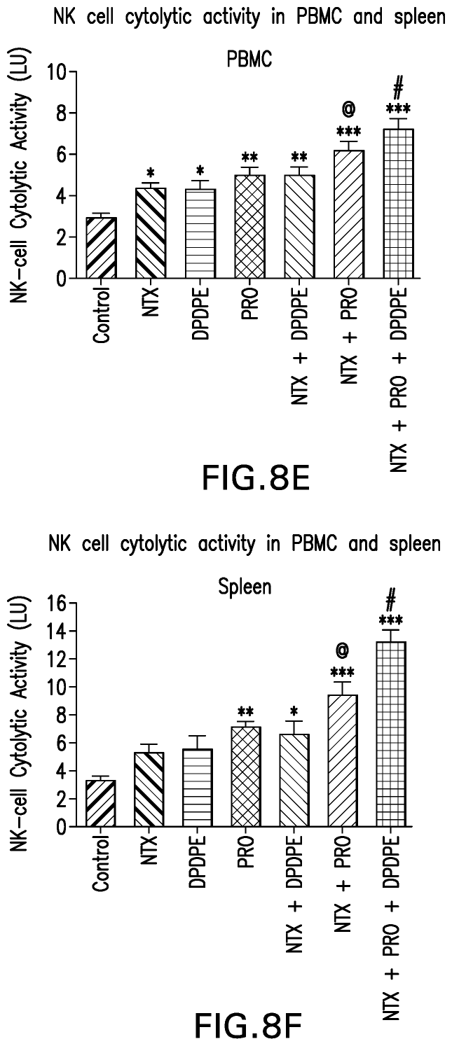

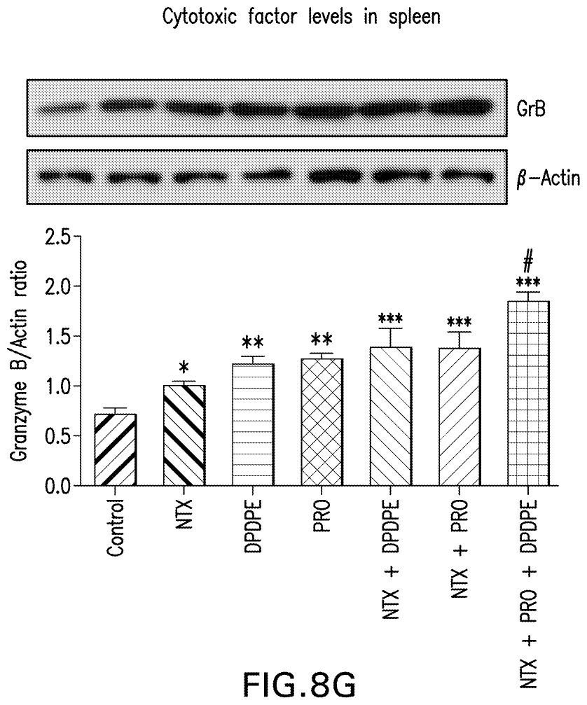

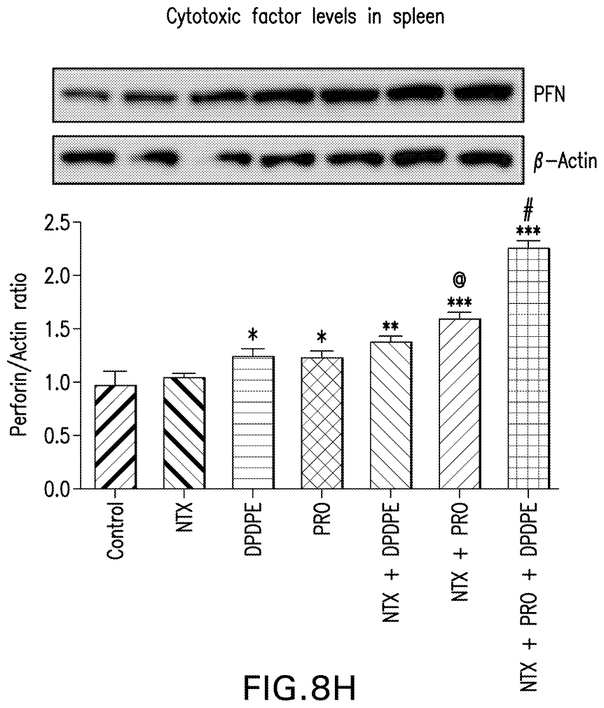

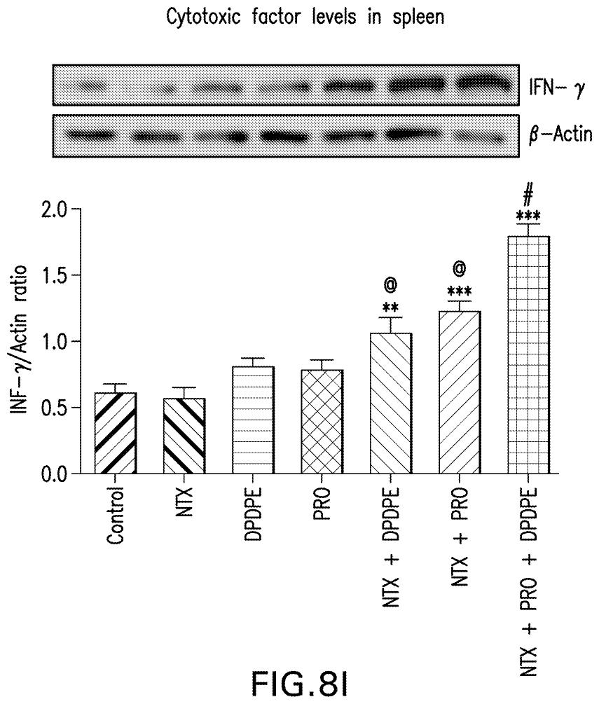

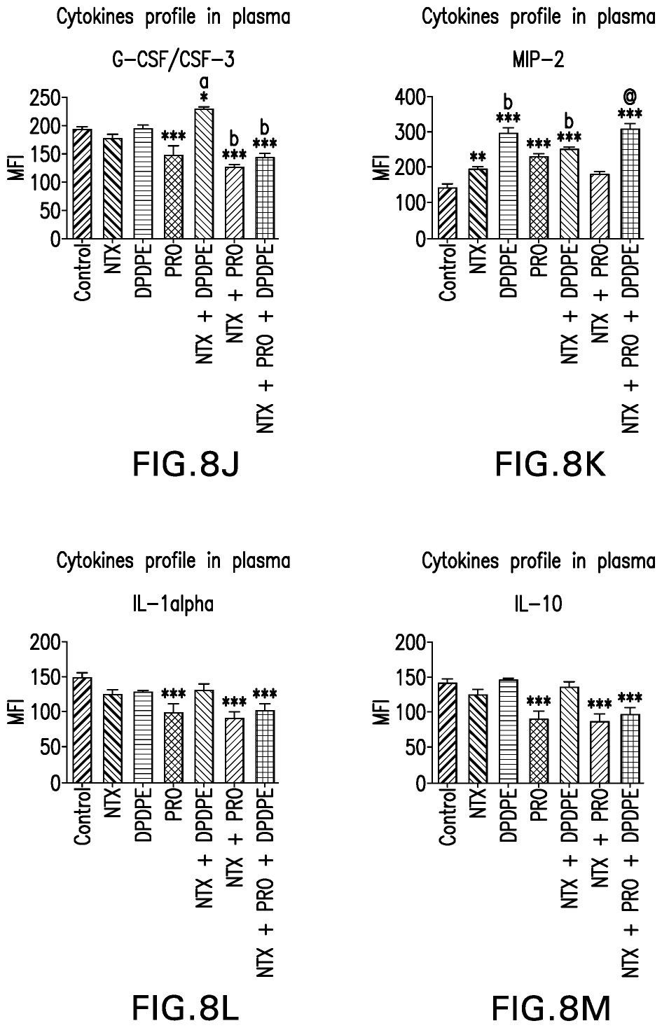

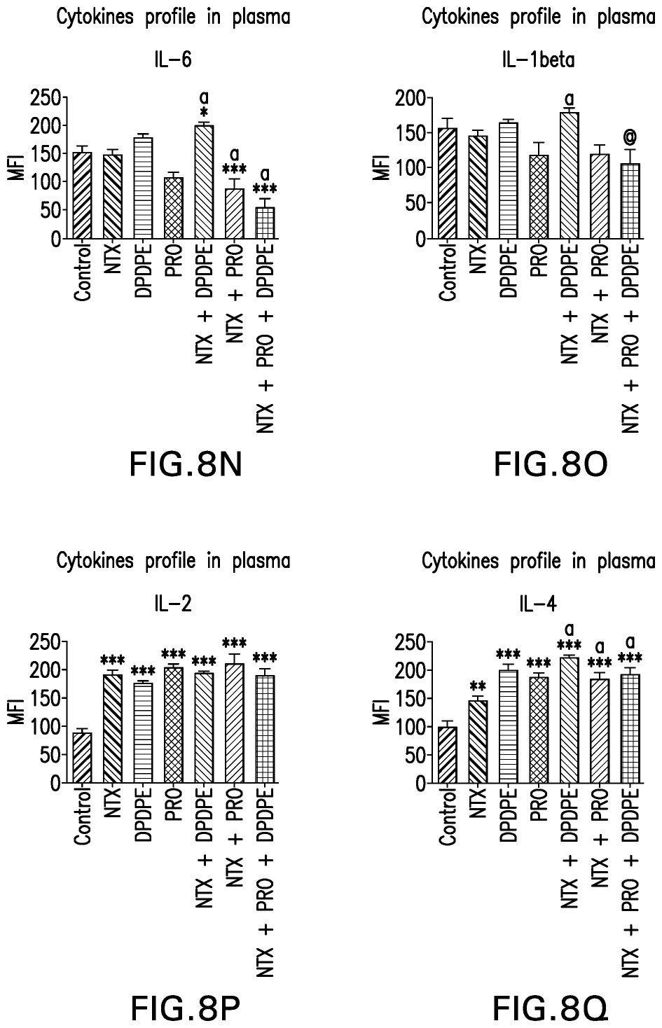

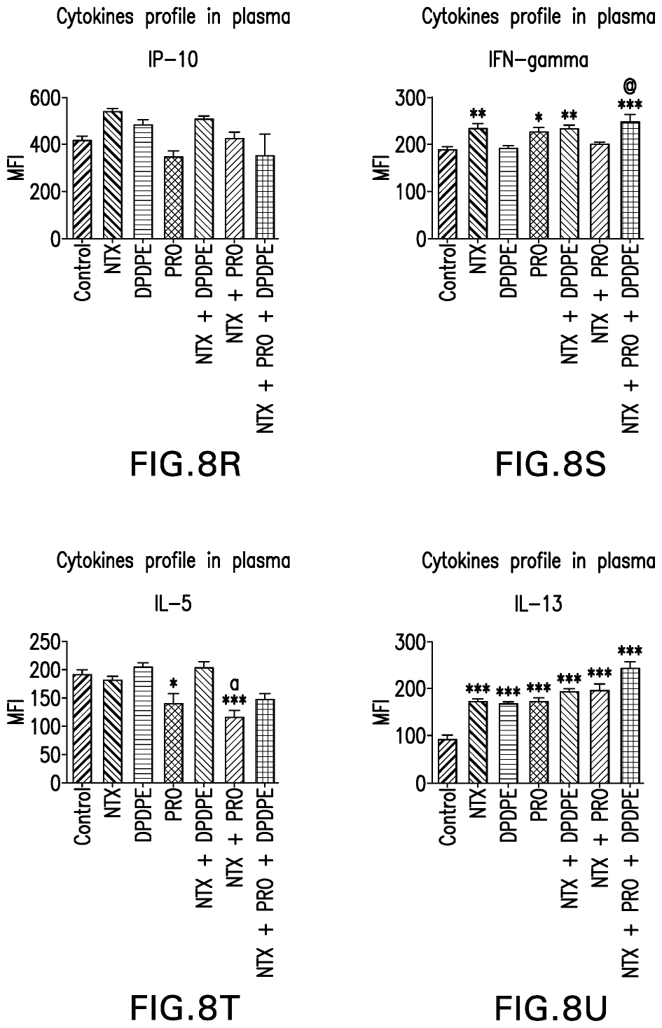

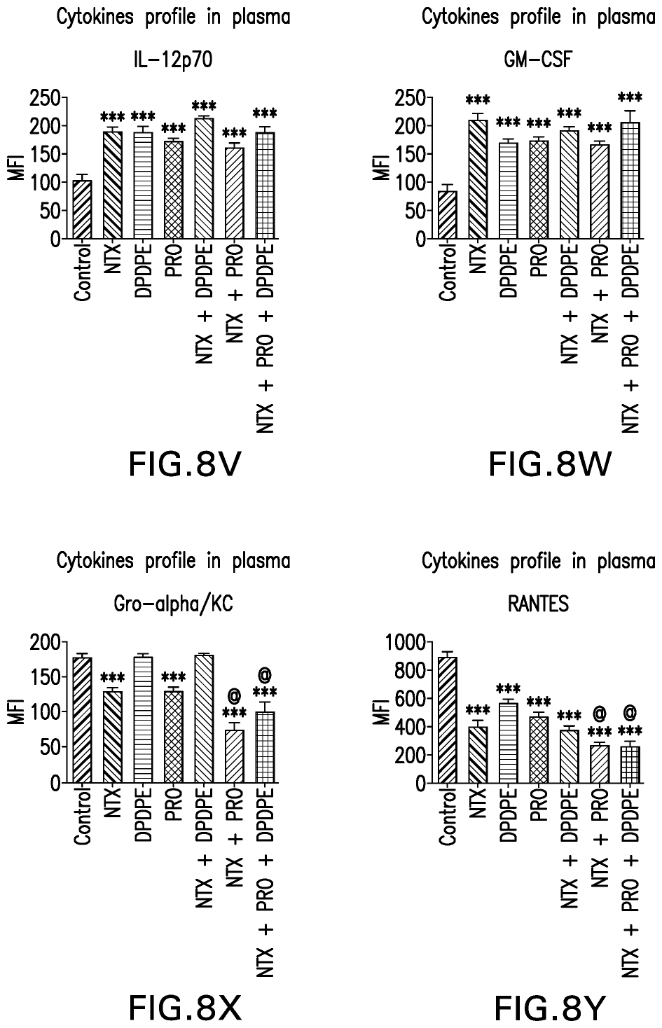

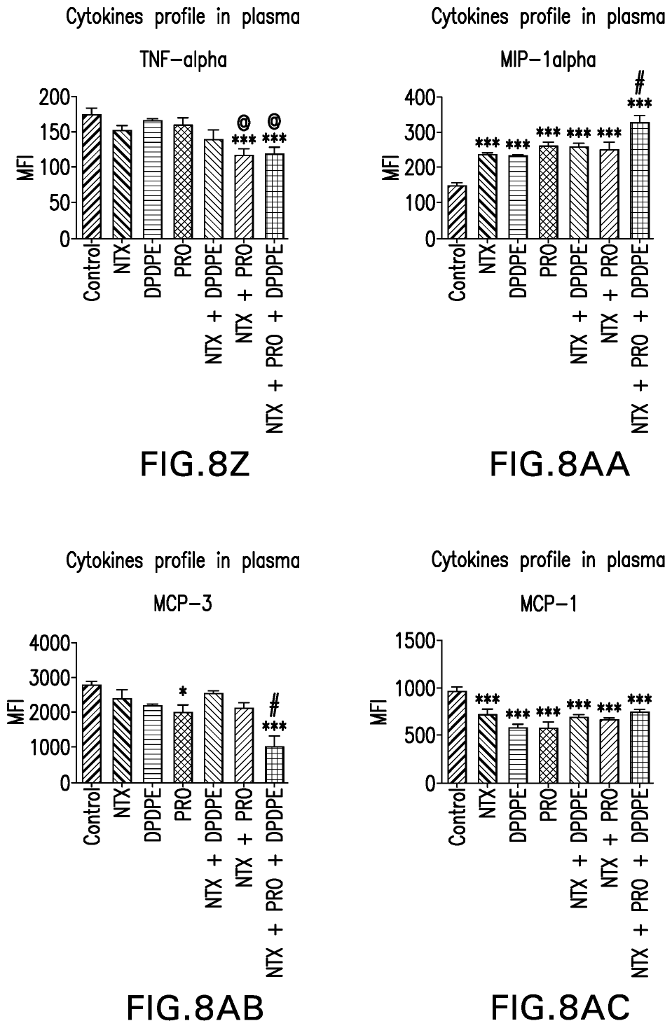

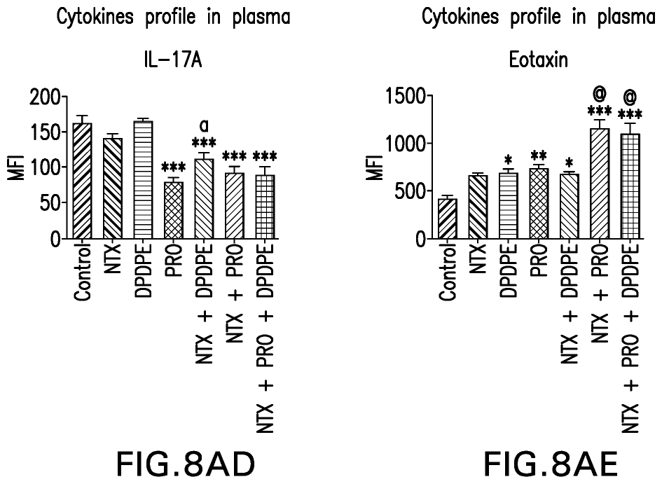

The tumor xenograft study was conducted in athymic nude rats, and therefore the major immune defense mechanism against cancer is controlled by NK cells in this host (26). Hence, it was determined the effects of beta-adrenergic and opioidergic drugs on NK cell numbers in peripheral blood mononuclear cells (PBMC) and cytolytic functions in PBMC and in the spleen as well as the levels of cytotoxic regulatory proteins in the spleen, which is considered an important immune organ where immune cells reside and differentiate depending on the inflammatory/anti-inflammatory conditions. The effects of these drugs on monocyte cell numbers in PBMC were measured, since these immune cells are known to mobilize from the bone marrow and spleen in response to chemotactic signals and to be recruited to tumor tissues guiding their further differentiation to macrophages. Tumor-associated macrophages, a very heterogeneous cell population in terms of phenotype and pro-tumor function, support tumor initiation, local progression, and distant metastasis (32). PBMC and spleen tissue of xenografted rats treated with different treatments and control animals were used for screening immune cell populations by flow cytometry to quantitate percentage (%) of CD161a⁺ NK cells in PBMC (FIG. 8A), CD11b⁺ monocytes in PBMC (FIG. 8B), CD161a⁺ NK cells in spleen (FIG. 8C), and RT1B⁺ macrophages in spleen (FIG. 8D). Treatment with NTX, DPDPE, or PRO alone increased NK cell numbers in PBMC and spleen. The effects of these drugs were potentiated when drugs were combined. The maximal increase in the NK cell numbers was observed when animals were given three drugs together. In contrast, monocyte numbers in PBMC were decreased following NTX+PRO or NTX+DPDPE+PRO treatments and macrophage numbers in spleen were reduced following PRO, NTX+DPDPE, NTX+PRO and NTX+DPDPE+PRO treatments.

The changes in the cytolytic activity of PBMC-derived and spleen-derived NK cells following the drugs treatments are shown in (FIGS. 8E-F). NK cell cytolytic activity was elevated by PRO treatment, and this effect of PRO was magnified when combined with NTX or NTX and DPDPE. The triple-drug combination (NTX+PRO+DPDPE) produced an about threefold increase in NK cell cytolytic activity of the PBMC. The beta-adrenergic and opioidergic drugs similarly affected the NK cell cytolytic activity of the spleen of the tumor xenograft host. The triple-drug combination produced an about fourfold enhancement of NK cell cytolytic activity in the spleen. Measurements of the levels of cytolytic proteins (granzyme B, perforin, and IFN-γ) in NK cells enriched from the spleen of the host animal also showed stimulatory effects of beta-adrenergic and opioidergic drugs (FIGS. 8GG-I). The combination of NTX with DPDPE or PRO produced an about twofold increase, while the triple-drug combination produced an about threefold increase in the levels of these cytotoxic proteins in NK cells of the spleen.

Cytokines are known to be critical autocrine and paracrine factors in tumor development, which are secreted into the tumor microenvironment to recruit and activate various inflammatory cells for promotion evasion from immune destruction (33). Measurements of cytokine levels in the plasma of these rats indicated that PRO inhibited a large number of inflammatory cytokines and chemokines such as G-CSF/CSF-3, IL-1 alpha, IL-10, IL-6, IL-5, Gro-α, TNF-α, MCP-3, and IL-17A (FIGS. 8J, 8L, 8M, 8N, 8T, 8X, 8Z, 8AB, and 8AD). The combination of the beta-adrenergic drug with NTX and/or DPDPE showed a greater inhibition of these inflammatory cytokines and chemokines, especially IL-6 (FIG. 8N), well known as a cytokine overrepresented in different types of cancer (34), which was at its lowest mean level after the treatment with NTX+PRO+DPDPE. The chemokines RANTES and MCP-1 (FIGS. 8Y and 8AC) were also inhibited by all three different drugs PRO, NTX, and DPDPE with a lowest mean observed for RANTES when they were combined as compared to control. On the contrary, the different combination treatments of PRO, NTX, and DPDPE increased the concentration in the plasma of cytokines involved in the response of NK cells: IL-2, IL-4, IL-13, and IL-12p70 (FIGS. 8P, 80, 8U, 8V). IL-2, IL13, and IL-12p70 showed similar increased responses to all drugs treated singly or in combination. Higher mean level of IL-4 was seen after the combined drug treatments. Also, NK cell activator IFN-γ (35) is elevated in the plasma of the rat treated by PRO or NTX compared to the control with the higher mean level reached for the triple treatment group (FIG. 8S). Finally, the macrophage inflammatory proteins MIP-2 and MIP-1α (FIGS. 8K, 8AA), granulocyte-macrophage colony-stimulating factor GM-CSF and Eotaxin (FIG. 8W, 8AE) were shown to be elevated in most of the treatment groups with the highest mean level achieved by treatment of NTX+PRO+DPDPE.

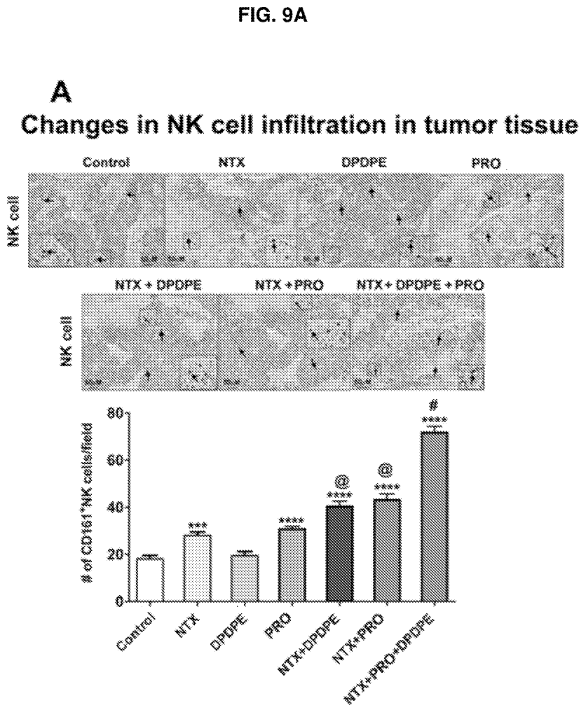

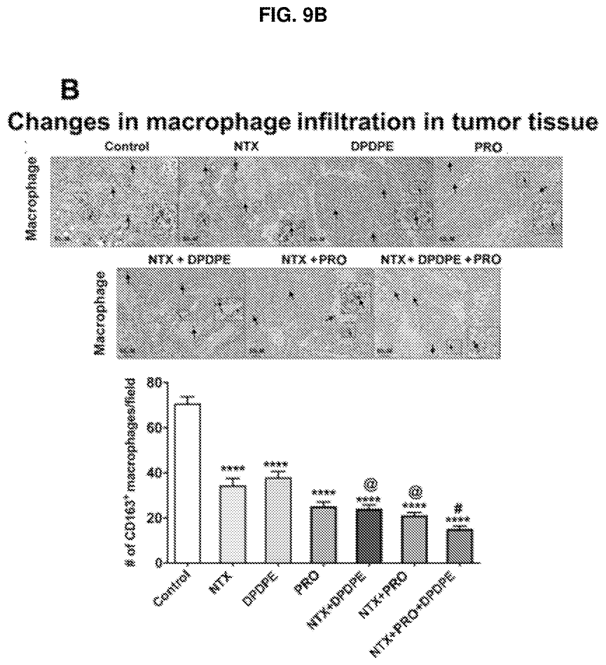

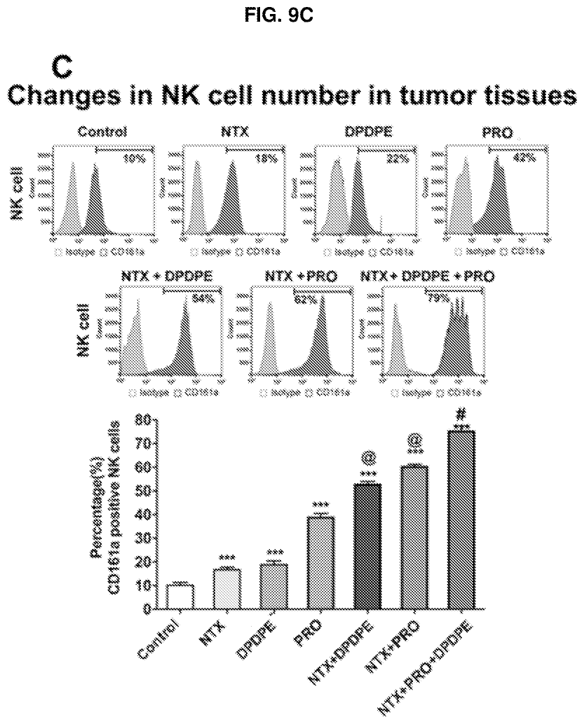

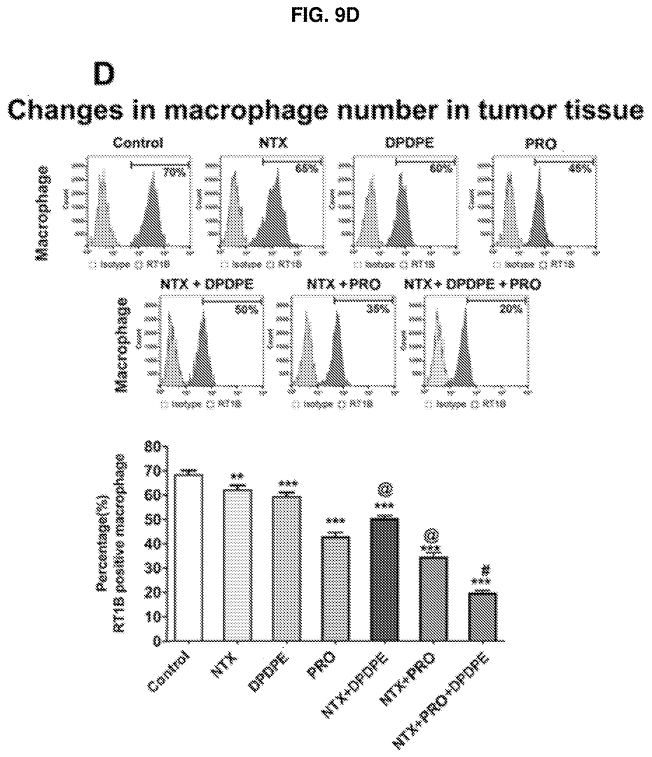

Further, for an in-depth understanding of how NTX, DPDPE, and PRO alone or in combination modulates the immune cell populations in the tumor microenvironments and contributes to the antitumor activities, immunocytochemical analysis of NK cells and macrophages infiltration in the tumor tissues (FIGS. 9A-B) and flow cytometry analyses of NK cells and macrophages number in tumor tissues (FIGS. 9C-D) was done. Both immunocytochemical data and flow cytometry analysis show that NTX, DPDPE, or PRO alone or NTX+DPDPE, NTX+PRO, or NTX+DPDPE+PRO combinations increased the percentage of CD161⁺ NK cells while they decreased the percentage of CD163⁺ macrophages at varying degrees in the tumor tissues. Like the effects observed for peripheral immunity, the maximal effects on immune cells in the tumor microenvironment by the treatment of NTX+PRO+DPDPE were also observed

Overall, these results demonstrate that single-agent and combination treatments enhance the influx of NK cells and decrease the accumulation of inflammatory macrophages in the tumor microenvironment and thereby possibly enhance the antitumor efficiency in addition to the drugs' direct antiproliferative effects on cancer cells.

### Example 9

### Effects on Apoptotic Regulatory Protein Levels and Signaling Pathway in Cancer Cells

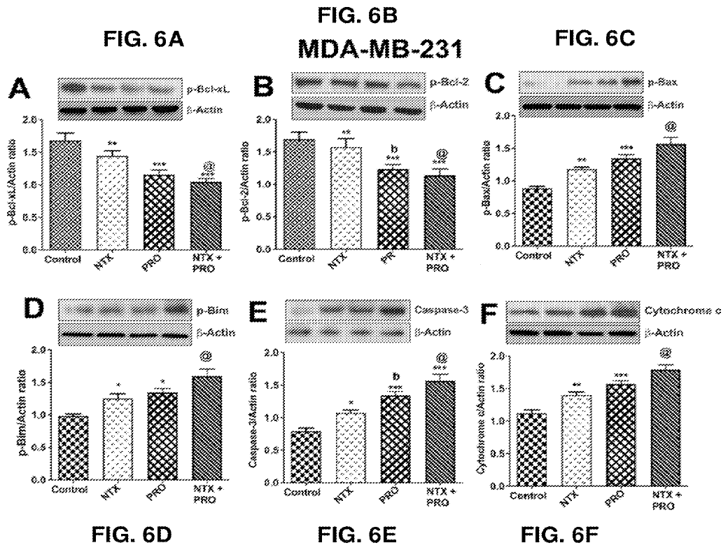

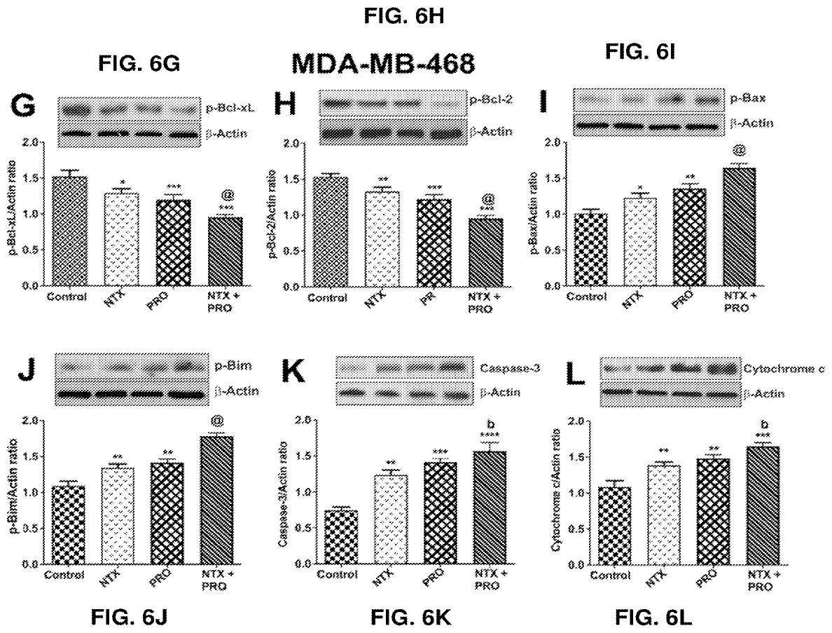

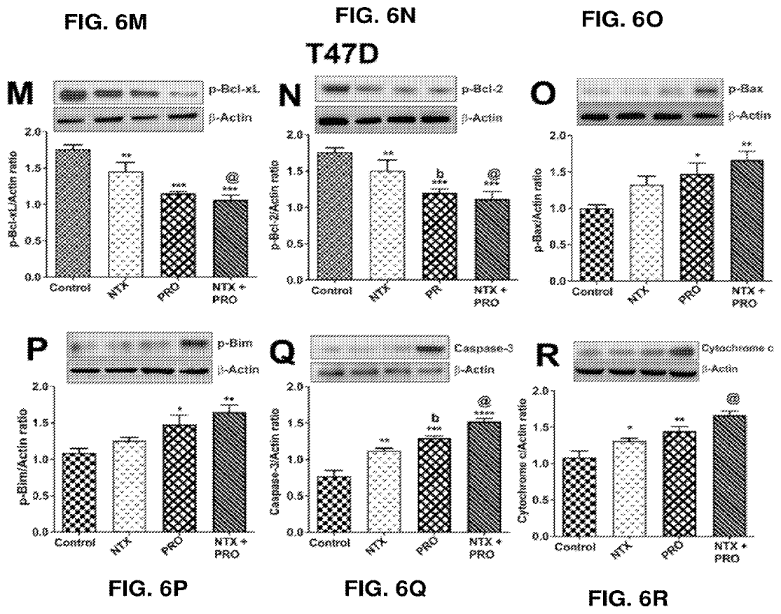

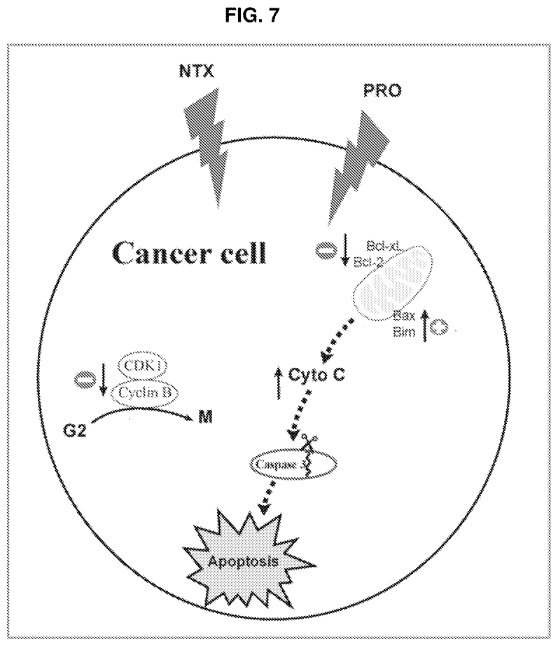

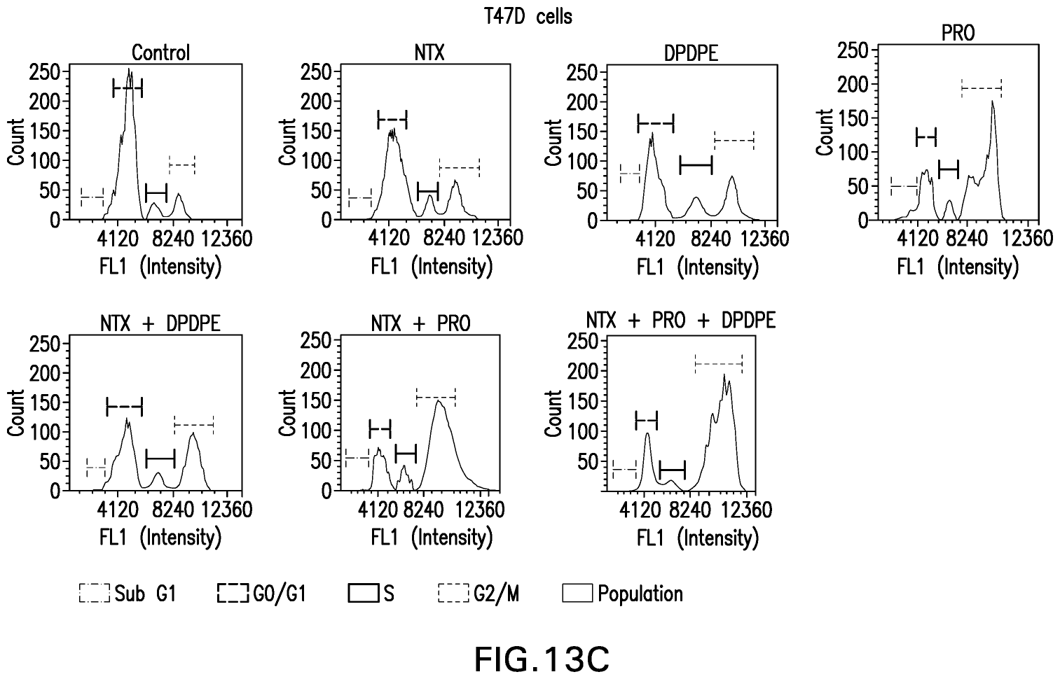

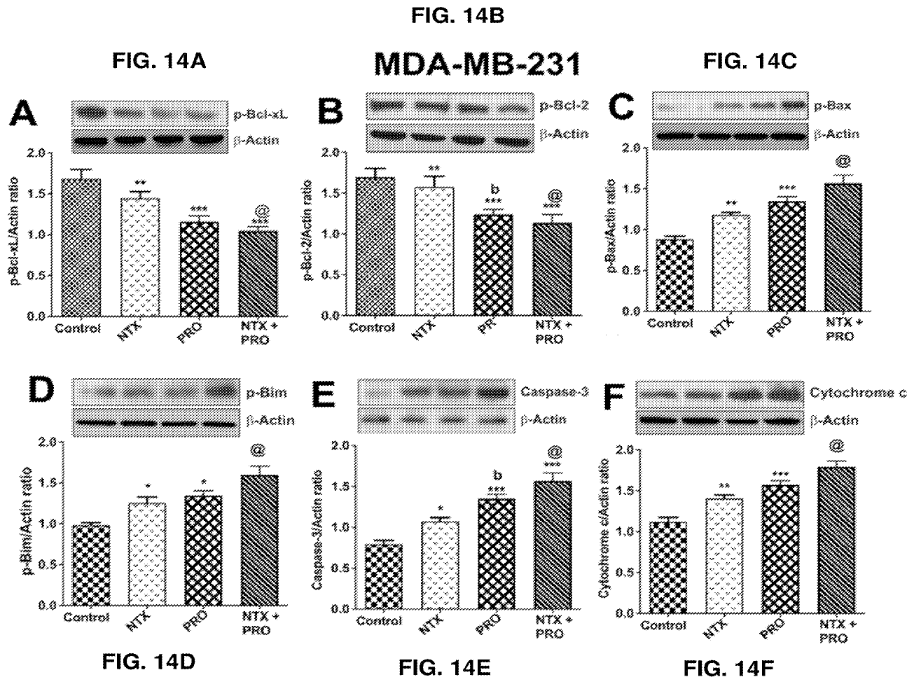

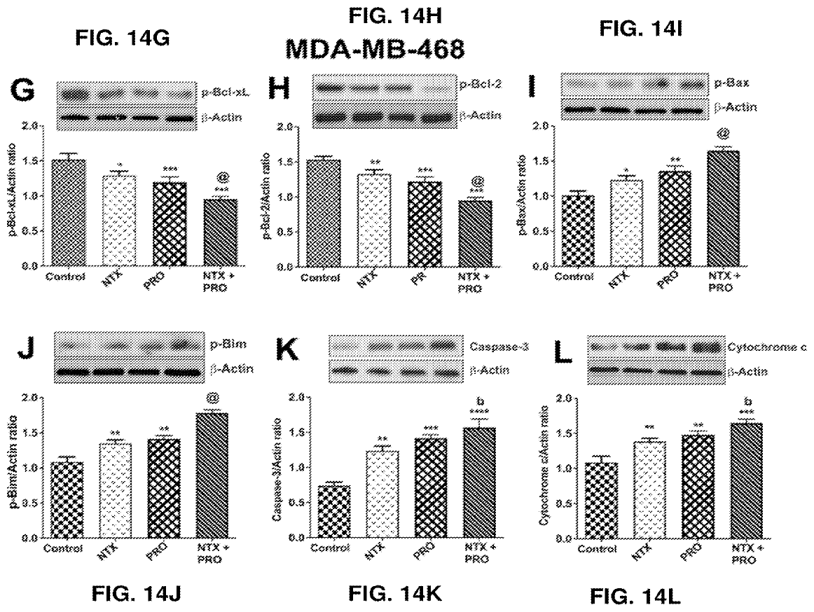

Because NTX and PRO promote breast cancer cells to undergo cell cycle arrest in G2/M, the following experiment was carried out to determine if these cells may be undergoing cellular death (23). MDA-MB-231, MDA-MB-468, and T47D cells were treated with a 100-μM concentration of NTX and PRO drugs alone or in combination or vehicle alone for 48 hours. After the treatment period, cell were extracted and used for Western blot analysis of anti-apoptotic p-Bcl-xL (FIGS. 6A, 6G, 6M) and p-Bcl2 (FIGS. 6B, 6H, 6N), effector proteins such as p-Bax (FIGS. 6C, 6I, 6O), pro-apoptotic molecule p-Bim (FIGS. 6D, 6J, 6P), effector caspase 3 (FIGS. 6E, 6K, 6Q) and Cytochrome c (FIGS. 6F, 6L, 6R) levels. The immunoblot results showed that the anti-apoptotic BCL-2 family isoforms such as Bcl-xL (FIGS. 14A-C), p-Bcl-xL (FIG. 6A, 6G, 6M), Bcl-2 (FIGS. 14D-F), and p-Bcl-2 (FIGS. 6B, 6H, 6N) were downregulated by NTX, PRO, and NTX+PRO treatment, whereas treatment of PRO or NTX or a combination of these drugs increased the levels of effector proteins such as Bax (FIGS. 14 G-I) and p-Bax (FIGS. 6C, 6I, 6O) and pro-apoptotic molecules like p-Bim (FIGS. 6D, 6J, 6P). In addition, effector caspase 3 (FIGS. 6E, 6K, 6Q), cleaved caspase 3 (CC3; FIGS. 14J-L). Representative immunoblots are presented on the top in FIG. 6, and mean densitometric values are presented as ratio of R-actin in the histograms. Data presented are mean±SEM (n=5-6 samples/group) and were analyzed using one-way ANOVA with the Newman-Keuls multiple comparisons post hoc test. *, p<0.05, **, p<0.01, ***, p<0.001 compared to control, b, p<0.05, compared with NTX. @, p<0.05, compared with the rest of the groups. A schematic illustration of the proposed mechanism of action of naltrexone and propranolol on induction of apoptosis in human breast cancer cells is provided in FIG. 7.

Importantly, NTX, PRO, and NTX+PRO treatment increased the levels of Cytochrome c (FIGS. 6F, 6L, 6R), which is known to play an important role in initiating apoptosis. The effects of these drugs on apoptotic regulatory proteins were enhanced when they were combined than when they were given singly by inducing mitochondria-mediated apoptosis. The results show that in breast cancer cells, NTX and PRO treatment causes G2/M phase cell cycle arrest by downregulation of CDK1, phosphorylation of CDK1 (activity) and cyclin B1 levels, up regulation of pro-apoptotic p-Bim, and effector proteins such as Bax and p-Bax and increased the levels of effector caspase 3 and CC3. Treatment of NTX and PRO caused increased levels of Cytochrome c, thereby causing apoptosis (FIG. 7).

### Discussion

PRO, NTX, and DPDPE inhibited the viability of basal MDA-MB-231 and MDA-MB-468 and luminal T47D breast cancer cells and showed additive effects when they were combined. Through in vitro studies with three different breast cancer-derived cells, it was shown that combined treatment was more effective in decreasing cell proliferation, migration, and invasion in all tumor cell types. In vivo studies using MDA-MB-231 cells xenotransplants confirmed the combination of NTX, DPDPE, and PRO reduced tumor growth, prevented metastasis, and increased the survival of the animal. Both in vitro and in vivo data show that these beta-adrenergic and opioidergic agents prevent tumor growth and metastasis by partly acting on tumor cells to induce G2/M cell phase arrest and cellular apoptosis as well as by increasing immune surveillance possibly by activating NK cell cytolytic functions and reducing systemic levels of inflammatory cytokines.

The data presented in the Examples provides evidence supporting the use of PRO as an anti-metastatic agent in breast cancer, particularly effective in the neo-adjuvant period or as a perioperative therapy (36). PRO used in early-stage breast cancer is associated with reduction of ki67 indices (37). Data from a neoadjuvant exposure trial in breast cancer on PRO+COX2 inhibitor also showed effects on EMT and immune microenvironment (38). Additionally, significant innervation of sympathetic nerves has been documented in human breast cancer (39). However, PRO is a non-specific P2 adrenergic blocker and acts via GPCRs, which are known to heterodimerize with closely related members resulting in the modulation of their functions (17). Evidence for a physical interaction between a B2AR and DOR has been documented (18, 19). It has been shown that DORs can form heteromeric complexes with B2ARs and increase the trafficking properties and decrease the signal transduction of B2ARs. Also, low doses of selective DORs can inhibit norepinephrine-mediated functions, possibly due to the activation of opposing pathways (opioids decrease intracellular cAMP and β2 receptors increase cAMP) and the physical regulation of these receptors (18, 19, 40). Here, it is shown that DOR agonist DPDPE potentiated B2AR antagonist PRO effects on breast cancer cells.

Opioid receptors have been shown to control breast cancer cell growth and proliferation (14, 41). Opioid receptors can also form heteromeric complexes between opioid receptor subtypes. For example, DOR can form heteromeric complexes with MOR, and NTX, a MOR antagonist, increases DPDPE, a DOR agonist's effects in immune and cancer cells (13, 14). The present application has shown that DPDPE was more effective in reducing breast cancer cell proliferation, migration, and invasion and NK cell functions when combined with NTX. Without being limited to a specific theory, the heteromeric interaction between B2AR, MOR, and DOR may also explain why PRO is more effective in reducing tumor growth, preventing metastasis, and increasing the survival of the animal with breast cancer when it is combined with NTX and DPDPE.

These Examples have identified that PRO significantly stimulates NK cell functions, and NTX and DPDPE promotes PRO effects on expanded NK cells from PBMCs of humans and from the spleens and PBMCs of tumor xenografted animals. Previously, it had been shown that the stimulatory guanine-nucleotide-binding protein GS coupled to B2AR is involved in transducing signals which inhibit NK cell lysis of tumor cells (42). Also, it has been shown that opioid receptors constitutively produce and function in rat spleens and PBMC-derived NK cells (13, 14) and human PBMC-derived NK cells (12). In the rat, NTX suppresses MOR to stimulate PBMC- and spleen-derived NK cell cytolytic functions. DOR agonist DPDPE also potentiates NTX stimulatory action on NK cell activity in rats (13, 14). Without being limited to a specific theory, the heteromeric interaction between B2AR, MOR, and DOR may also explain why PRO is more effective in inducing NK cell cytolytic functions when it is combined with NTX and DPDPE.

It has been observed that PRO, NTX, and DPDPE increased levels of cytokines involved in the modulation of NK cells while they reduced levels of Th1 inflammatory cytokines in blood in the xenograft animals. The data in these examples agree with the findings that PRO increases cytokines IL-2, IL-4, IL-12, IL-17, and IFN-γ in animal models of breast cancer (43). Propranolol was shown to increase survival of tumor-bearing mice and to increase IL-2, IL-4, IL-13, IL-12p70, and IFN-γ cytokines (44). The Examples in the present application have confirmed the activation effect of PRO on these cytokines, involved in the modulation of NK cells (33), and demonstrated that the combination of PRO with NTX and DPDPE potentiated the increased levels of these cytokines. Many pro-inflammatory cytokines are overexpressed in breast cancer and associated with a poor prognosis and a large impact on the latest stage of cancer such as angiogenesis and metastasis (45, 46). The Examples have shown a diminution of the plasma level of many pro-inflammatory cytokines such as G-CSF/CSF-3, IL-1 alpha, IL-10, IL-6, IL-5, Gro-α, TNF-α, MCP-3, and IL-17A, suggesting a less aggressive cancer profile following PRO, NTX, and/or DPDPE treatments. Because the xenograft animal models used were athymic rats and they lack T-cell function, the effects of these drugs on Th2 cytokines cannot be properly determined in this animal model. However, strong inhibitory effects of PRO and NTX were observed on plasma levels of inflammatory cytokines, which agree with previous findings that these agents reduce pro-inflammatory cytokines (47, 48).

The data from these Examples also identified significant changes in the tumor immune microenvironment. An increase in the number of antitumor NK cells but a decrease in the number of protumor monocytes/macrophages in the spleen, PBMC, and tumor tissues in association with a reduction in tumor volume in PRO-, NTX-, and/or DPDPE-treated animals was found. After their recruitment into the tumor tissue, monocytes are known to differentiate into tumor-associated macrophages (TAM), a very heterogeneous cell population in terms of phenotype and protumor function, and support tumor initiation, local progression, and distant metastasis (49). Classically activated macrophages (M1), following exposure to interferon, have antitumor activity and elicit tissue destructive reactions; however, in response to inflammatory cytokines, macrophages undergo alternative activation (M2) and gain protumor activity (50). Therefore, beta-adrenergic and opioidergic agents significantly influenced immune cell infiltration and tumor invasion to promote antitumor function.

In summary, these Examples demonstrated that the combination treatment of PRO, NTX, and DPDPE produces marked reduction in growth and progression of breast cancer in a preclinical animal model via direct action on cancer cell proliferation and epithelial to mesenchymal transition and via the enhancement of the host's immune functions for tumor invasion and killing and this combination therefore has therapeutic value for the treatment of cancers, such as breast cancer.

### Example 10

### Chemical and Reagents

Anti PD-1 mAb (RMP1-14) and rat IgG2a isotope control antibody (2A3) were purchased from BioXcell (West Lebanon, NH). Naltrexone and Propranolol were purchased from Sigma Aldrich (St. Louis, MO, USA).

### Colon Cancer Cell Cultures

Mouse CT26 colon cancer cells (RRID: CVCL_YJ79) were obtained from American Type Culture Collection (ATCC; Rockville, MD), and maintained in cultures with the RPMI-1640 medium suggested by the supplier at 37° C. in a humid environment containing 5% CO₂.

### Animal Maintenance

Male and female BALB/c mice aged 43-49 days old were purchased from Charles River (Charles River, Portage, MI) and maintained in a pathogen-free condition with a 12-hour light/dark cycle at Animal Research Facility of our institute. Animal care was done in accordance with institutional guidelines and complied with NIH policy.

### Establishment of Subcutaneous Tumors

For tumor growth study, Mouse CT26 colon cancer cells were grown until about 90% confluence just 1 day before injection. On the day of injection, after checking cell viability with trypan blue, CT26 colon cancer cells were diluted in PBS at a final concentration of 1×10⁶ cells/rat in 200-μl of PBS-50% Matrigel (BD Biosciences, San Jose, CA) mixture and injected subcutaneously (SC) into the right flank of the rats. After tumors reach a diameter of approximately 50 mm³, animals (n=6) were randomly assigned to different treatment groups as follows. Control (PBS+2A3, 200 μg/kg), Anti-PD-1 mAb (12.5 mg/kg q week IP), Naltrexone (10 mg/kg, subcutaneously, sc), Propranolol (10 mg/kg, sc), Propranolol (10 mg/kg; sc)+Naltrexone (10 mg/kg, sc), or Anti-PD-1 mAb (12.5 mg/kg IP, qweek)+Propranolol (10 mg/kg, sc)+Naltrexone (10 mg/kg, sc). Tumor shrinkage/growth was measured in animals daily and animal weights were measured every other day. Animals were euthanized when the tumor reaches 2000 mm³ and early euthanasia was performed if they displace signs of distress or 10% loss of body weight. Three dimensions of tumor were measured using electronic calipers, and tumor volumes were calculated by the formula as, L×W²/2. The mean±SE of tumor volume was calculated weekly for each experimental group and the data are expressed as mean absolute tumor volume. After animal sacrifice, the subcutaneous tumors were excised intact from rats to determine the tumor weight in grams and photographed to visualize differences in tumor morphology and compare size. A portion of excised tumors was snap-frozen by immersion in liquid nitrogen and stored at −80° C. until further use.

Enrichment of NK Cells from Spleen and PBMC

After the end of drug treatment period, the spleen tissues from control and drug treated rats was processed (53). RBCs and granulocytes were removed from splenocyte suspensions by density centrifugation using Histopaque 1083 (Sigma-Aldrich). Splenocytes (˜10×10⁷ cells per spleen) were extracted from the middle layer, washed with RPMI-1640 (Gibco), and resuspended in buffer (PBS, 0.5% BSA). Dead cells were removed using dead cell removal Kit (Miltenyi Biotec, catalog #130-090-101) and further NK cells were enriched using a cocktail of biotin-conjugated antibodies and anti-biotin micro beads (Mouse NK Cell Isolation Kit, catalog #130-115-818) according to the manufacturer's instructions (Miltenyi Biotec). NK cells were then enriched by magnetic separation (negative selection), using an AutoMACS Magnetic Separator (Miltenyi Biotec). The enriched fraction consistently yielded ˜5×10⁶ cells per spleen with a purity of ˜80-90% (53) and enriched NK cells were used for cytotoxicity assays or lysis with appropriate buffer for protein analyses. Whereas peripheral blood mononuclear cells (PBMC) from mouse were isolated from freshly obtained heparinized whole blood using SepMate 15 ml tube (Stemcell Technologies, Vancouver, BC, Canada) according to the manufacturer instructions. The pelleted cells (PBMC) were counted in a hemocytometer and assayed for viability by Trypan blue exclusion. PBMC cell pellets from 3 animals in each group were pooled, washed three times with PBS and finally re-suspended in 2 ml RPMI medium containing 10% FBS and used for NK cell cytotoxicity.

### NK Cell Cytotoxicity

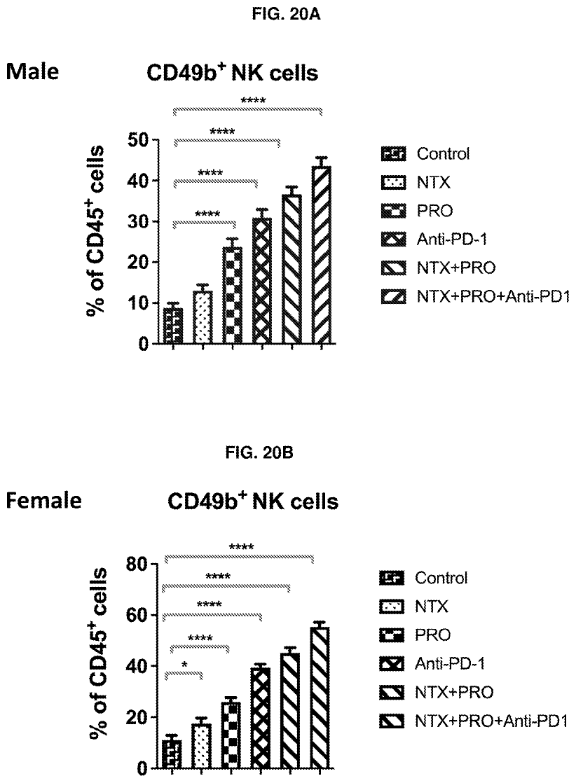

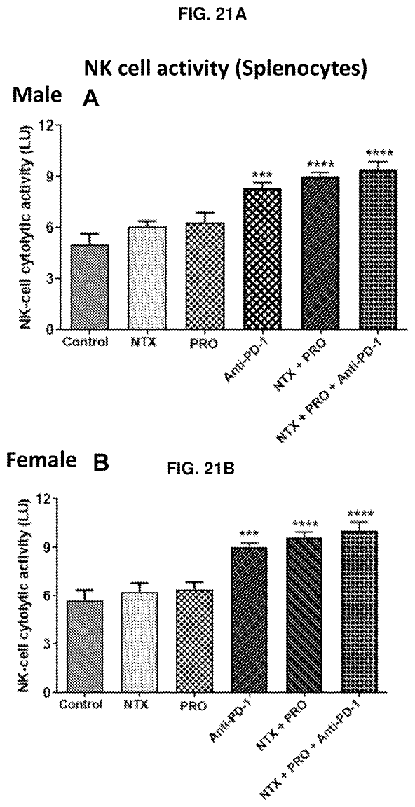

The cytotoxicity of enriched NK cells from PBMC and spleen against NK-sensitive YAC-1 (ATCC; Cat #TIB-160, Rockville, MD; RRID: CVCL 2244) target cell was determined by calcein AM assays (53). YAC-1 (murine lymphoma) cells were grown and maintained in RPMI 1640 without phenol red (Gibco), containing 1% penicillin-streptomycin (Gibco) and 10% FBS (Gibco). YAC-1 cells were washed and incubated with 5 mM calcein AM (Sigma-Aldrich) in serum-free RPMI-1640 for 10 min at 37° C. Labeled YAC-1 cells were washed and plated into U-bottom 96-well plates (Falcon) at a concentration of 5×104 cells per well. NK cells were added at E:T (20:1) ratio in triplicates. YAC-1 cells in RPMI alone were to determine spontaneous calcein AM release, whereas maximal release was achieved by lysing target cells in buffer (0.1% Triton X-100). Enriched NK cells from PBMC and spleen were preincubated for 12 h with IL-2 at 37° C. (100 ng/ml; R&D Systems) prior to 4-h incubation with YAC-1 target cells (53). All assays were analyzed and percent cytotoxicity for each sample was calculated. Data is presented in FIGS. 21A-B.

### Flow Cytometry Analysis of Immune Cells

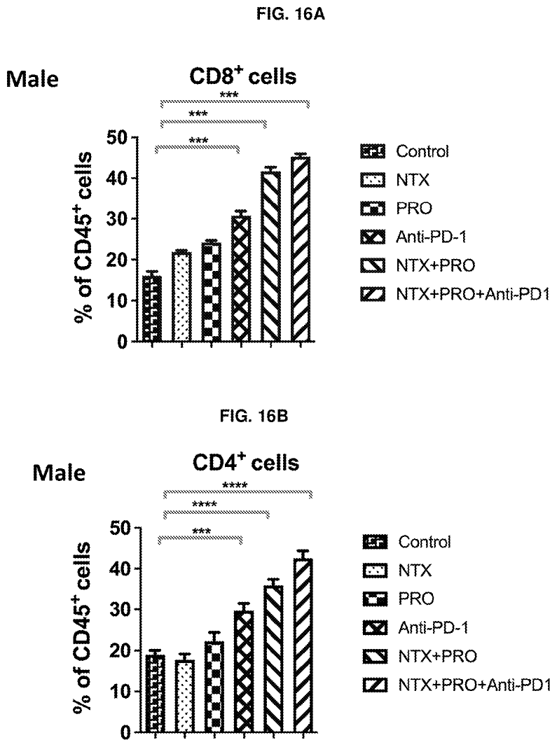

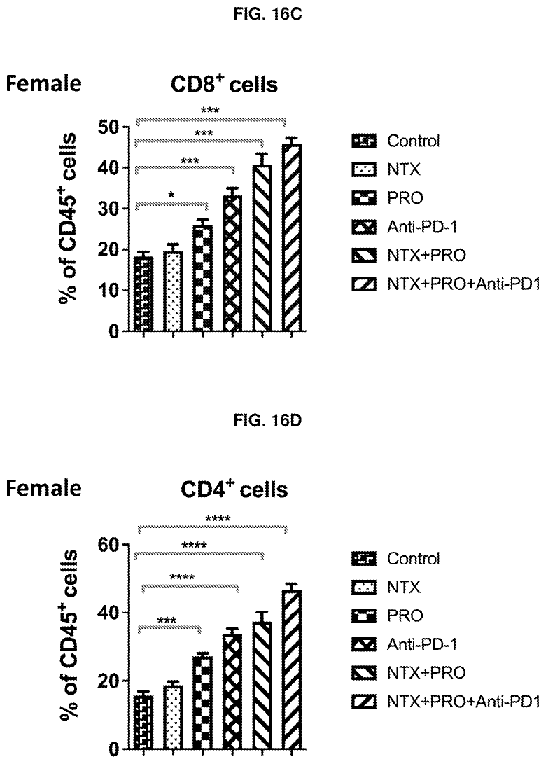

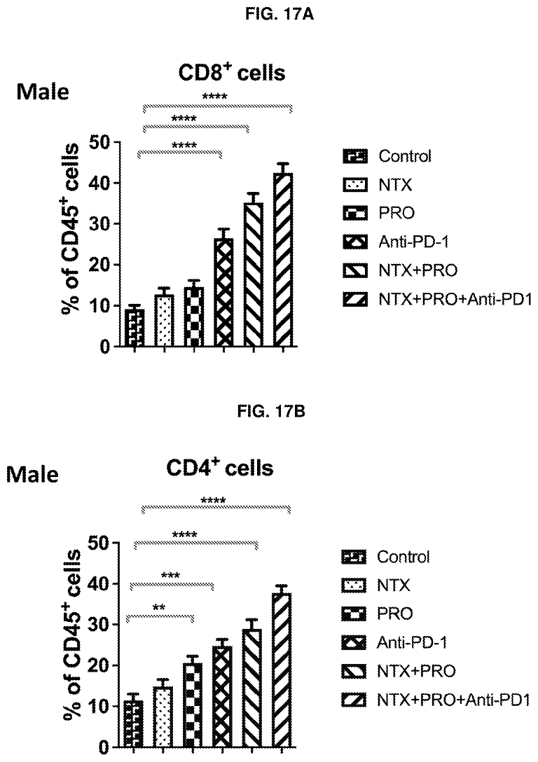

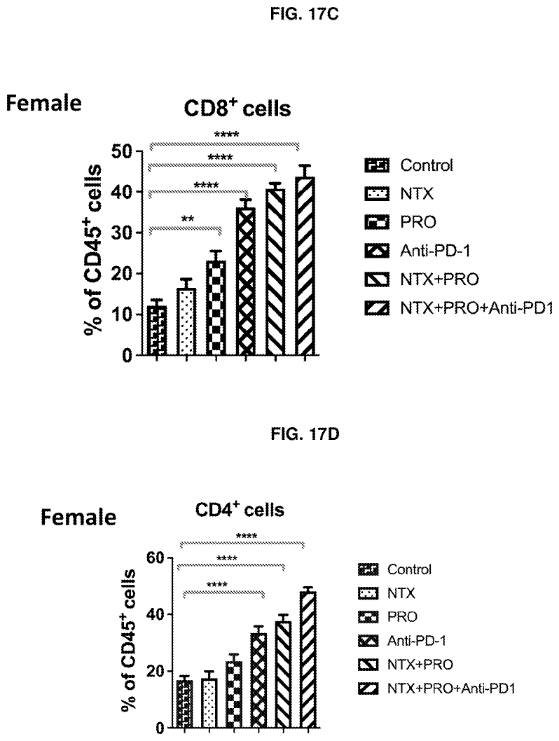

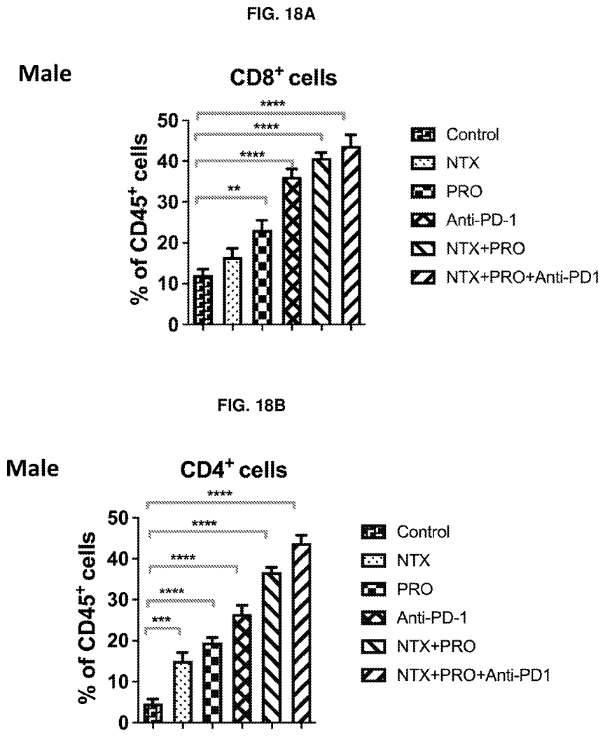

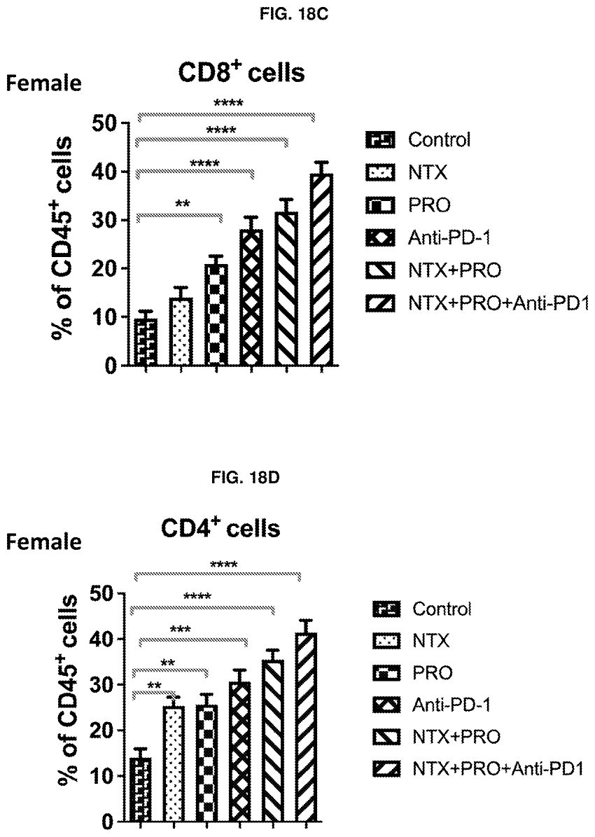

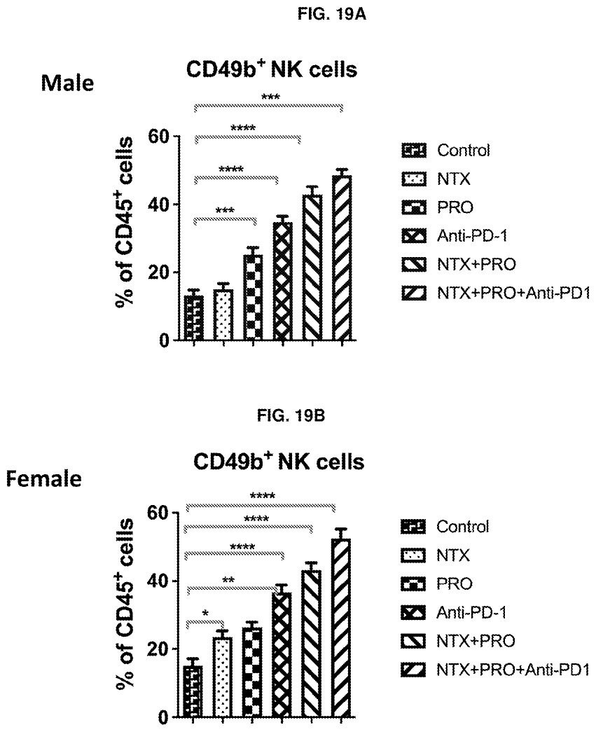

PBMC and splenocytes samples were prepared as described above. Tumor tissue was minced with a scalpel blade using a crisscross method and ground with 1 mL of ACK lysing buffer to remove red blood cells. The cell suspensions were filtered through a 70-micron sieve and then washed with PBS. Cells from the tumor were collected and dispersed with 100 μL of PBS and stained by the addition of fluorescence-conjugated antibodies. A list of antibodies, dye, isotype controls, and beads used for the flow cytometry experiment is given in Tables 2 and 3. Immune cells from the tumor, PBMCs, and splenocyte samples were examined by following the gating strategy (54). The data collected from each sample were exported and analyzed using FlowJo™ version 10.7 (BD Biosciences). The immune cells from PBMC, splenocytes and tumor cell suspensions were fluorescently stained with CD3-FITC, CD49b-APC, and Anti-NKp46-PE (A), and analyzed by flow cytometry using the Cytomics FC 500 flow cytometry analyzer (Beckman Coulter, Inc. Fullerton, CA). Cell debris and dead cells were excluded from the analysis based on scatter signals, CD45-VioGreen™, and 4′,6-diamidino-2-phenylindole (DAPI) fluorescence. Flow cytometry data is presented in FIGS. 16A-D and 20A-B (splenocytes), FIGS. 17A-D (PBMC), FIGS. 18A-D and 19A-D (tumor cells)

### Statistical Analysis

All the graphs, calculations, and statistical analyses were performed using GraphPad Prism software version 8.0 for Windows (GraphPad Software, San Diego, CA) and results are presented as mean±SD or mean±SEM. The significance between treatment and controls was assessed using one-way ANOVA between treatments and two-way ANOVA between treatments and days of exposure. P 0.05 was considered statistically significant.

### Results and Discussion

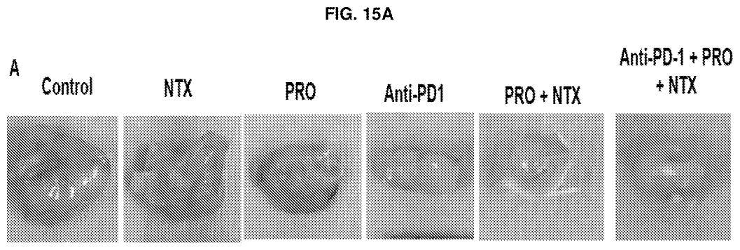

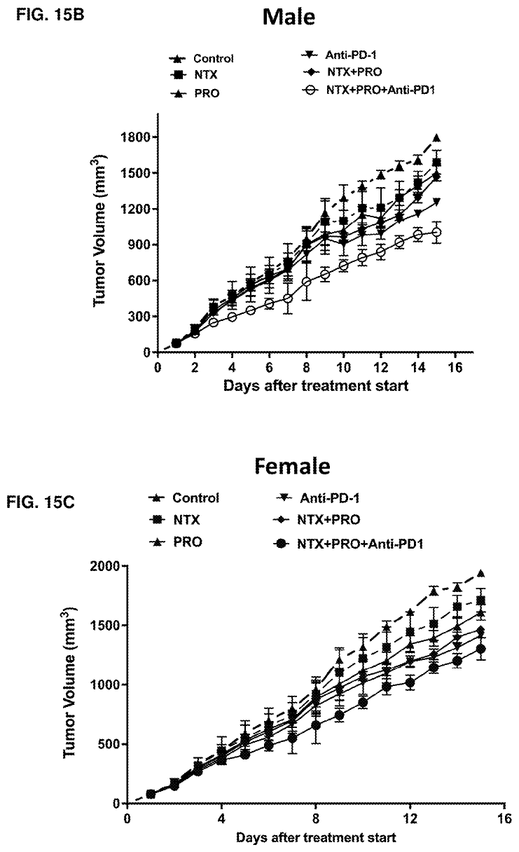

The data indicated that CT-26 tumor cells in mouse xenografts responded by decreasing tumor volume to PRO, NTX and anti-PD1 treatment alone and significantly potentiation of the drug effects were seen in combination treatment in both male and female recipient mice (FIG. 15A-C). Naltrexone and propranolol combination treatment was associated with CD8+ and CD4+ T cells and NK cell activation both peripherally and in the tumor microenvironment. Some of these immune parameters showed potentiation of the drug effects when combined with anti-PDX in both male and female recipient mice.

These data together shows that propranolol, a beta blocker used to treat high blood pressure; naltrexone, an opiate antagonist used for drug and alcohol dependence; and anti-PD1 when combined, produce marked inhibitory effects on tumor growth and tumor mass while increasing NK and T cell activities. The identify a novel treatment with a combination of approved classes of drugs in preclinical breast cancer models.

Many modifications and variations of this invention can be made without departing from its spirit and scope, as will be apparent to those skilled in the art. The invention is defined by the terms of the appended claims, along with the full scope of equivalents to which such claims are entitled. The specific embodiments described herein, including the following examples, are offered by way of example only, and do not by their details limit the scope of the invention.

All references cited herein are incorporated by reference to the same extent as if each individual publication, database entry (e.g., Genbank sequences or GeneID entries), patent application, or patent, was specifically and individually indicated to be incorporated by reference. This statement of incorporation by reference is intended by Applicants, pursuant to 37 C.F.R. § 1.57(b)(1), to relate to each and every individual publication, database entry (e.g., Genbank sequences or GeneID entries), patent application, or patent, each of which is clearly identified in compliance with 37 C.F.R. § 1.57(b)(2), even if such citation is not immediately adjacent to a dedicated statement of incorporation by reference. The inclusion of dedicated statements of incorporation by reference, if any, within the specification does not in any way weaken this general statement of incorporation by reference. Citation of the references herein is not intended as an admission that the reference is pertinent prior art, nor does it constitute any admission as to the contents or date of these publications or documents.

The present disclosure is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

The foregoing written specification is considered to be sufficient to enable one skilled in the art to practice the invention. Various modifications of the invention in addition to those shown and described herein will become apparent to those skilled in the art from the foregoing description and fall within the scope of the appended claims.

### REFERENCES (EXAMPLES 1-9)

1. National Cancer Institute. Cancer stat facts: female breast cancer. https://seer.cancer.gov/statfacts/html/breast.html. 2019.
2. K. T. Hwang et al., Impact of breast cancer subtypes on prognosis of women with operable invasive breast cancer: a population-based study using SEER database. Clin. Cancer. Res. 25, 1970-1979 (2019).
3. M D'Arcy et al., Race-associated biological differences among Luminal A breast tumors. Breast Cancer Res. Treat. 152, 437-448 (2015).
4. S. Burugu, K. Asleh-Aburaya, T. O. Nielsen, Immune infiltrates in the breast cancer microenvironment detection, characterization, and clinical implication. Breast Cancer 24, 3-15(2017).
5. C. N. Baxevanis, S. P. Fortis, S. A. Perez, The balance between breast cancer and immune system: challenges in prognosis and clinical benefit from immunotherapies. Semin. Cancer Biol., S1044-579X(19)30418-3, DOI: 10.1016/j.semcancer.2019.12.018 (2019).
6. S. K. Deshmukh et al., Inflammation, immunosuppressive microenvironment and breast cancer, opportunities for cancer prevention and therapy. Ann. Transl. Med. 7, 593 (2019).
7. D. K. Sarkar et al., Transplantation of β-endorphin neurons into the hypothalamus promotes immune function and restricts the growth and metastasis of mammary carcinoma. Cancer Res. 71, 6282-6291 (2011).
8. C. Zhang et al., Beta-endorphin cell therapy for cancer prevention. Cancer Prev. Res. (Phila.) 8, 56-67 (2015).
9. D. K. Sarkar, S. Murugan, C. Zhang, N. Boyadjieva, Regulation of cancer progression by R-endorphin neuron. Cancer Res. 72, 836-840 (2012).
10. D. P. Maher, D. Walia, N. M. Heller, Suppression of human natural killer cells by different classes of opioids. Anesth. Analg. 128, 1013-1021 (2019).
11. G. Shakhar, S. Ben-Eliyahu, In vivo beta-adrenergic stimulation suppresses natural killer activity and compromises resistance to tumor metastasis in rats. J. Immunol. 160, 3251-3258 (1998).
12. C. Pérez Piñero, A. Bruzzone, M. G. Sarappa, L. F. Castillo, I. A. Lüthy, Involvement of β- and β2-adrenoceptors on breast cancer cell proliferation and tumour growth regulation. Br. J. Pharmacol. 166, 721-736 (2012).
13. N. 1. Boyadjieva, K. Chaturvedi, M. M. Poplawski, D. K. Sarkar, Opioid antagonist naltrexone disrupts feedback interaction between mu and delta opioid receptors in splenocytes to prevent alcohol inhibition of NK cell function. J. Immunol. 173, 42-49 (2004).
14. D. K. Sarkar, A. Sengupta, C. Zhang, N. Boyadjieva, S. Murugan, Opiate antagonist prevents μ- and δ-opiate receptor dimerization to facilitate ability of agonist to control ethanol-altered natural killer cell functions and mammary tumor growth. J. Biol. Chem. 287, 16734-16747 (2012).
15. M. Dokur, N. Boyadjieva, D. K. Sarkar, Catecholaminergic control of NK cell cytolytic activity regulatory factors in the spleen. J. Neuroimmunol. 151, 148-157 (2004).
16. Gomes et al., G protein-coupled receptor heteromers. Annu Rev Pharmacol Toxicol. 56, 403-425 (2016).
17. S. Pepe, R. P. Xiao, C. Hohl, R. Altschuld, E. G. Lakatta, ‘Cross talk’ between opioid peptide and adrenergic receptor signaling in isolated rat heart. Circulation 95, 2122-2129 (1997).
18. B. A. Jordan, N. Trapaidze, I. Gomes, R. Nivarthi, L. A. Devi, Oligomerization of opioid receptors with beta 2-adrenergic receptors: a role in trafficking and mitogen-activated protein kinase activation. Proc. Natl. Acad. Sci. U.S.A. 98, 343-348 (2001).
19. R. Root-Bernstein, B. Churchill, M. Turke, U. K. T. Subhramanyam, J. Labahn, Mutual enhancement of opioid and adrenergic receptors by combinations of opioids and adrenergic ligands Is reflected in molecular complementarity of ligands: drug development possibilities. Int J Mol Sci. 20, 4137 (2019).
20. X. Dai, H. Cheng, Z. Bai, J. Li, Breast cancer cell line classification and Its relevance with breast tumor subtyping. J Cancer 8, 3131-3141 (2017).
21. N. A. Franken, H. M. Rodermond, J. Stap, J. Haveman, C. van Bree, Clonogenic assay of cells in vitro. Nat. Protoc. 1, 2315-2319 (2006).
22. C. R. Justus, N. Leffler, M. Ruiz-Echevarria, L. V. Yang, In vitro cell migration and invasion assays. J. Vis. Exp., e51046 (2014). doi: 10.3791/51046.
23. N. Nordin et al., In vitro cytotoxicity and anticancer effects of citral nanstructured lipid carrier on MDAMBA-231 human breast cancer cells. Sci Rep. 9, 1614 (2019).
24. C. F. A Warren, M. W. Wong-Brown, N. A. Bowden, BCL-2 family isoforms in apoptosis and cancer. Cell Death Dis. 10, 177 (2019).
25. L. C. Crowley, N. J. Waterhouse, Detecting cleaved caspase-3 in apoptotic cells by flow cytometry. Cold Spring Harb Protoc. 2016, 11 (2016).
26. H. P. Hougen, The athymic nude rat. Immunobiological characteristics with special reference to establishment of non-antigen-specific T-cell reactivity and induction of antigen-specific immunity. APMIS Suppl. 21, 1-39 (1991).
27. N. M. Villanueva, M. Sestelo, L. Meira-Machado, A method for determining groups in multiple survival curves. Stat Med. 38, 866-877 (2019).
28. L. Mezzanotte et al., In vivo bioluminescence imaging of murine xenograft cancer models with a red-shifted thermostable luciferase. Mol Imaging Biol. 12, 406-414 (2010).
29. M. Jursková, L'. Danihel, S. Polak, I. Varga, Ki67, PCNA and MCM proteins: markers of proliferation in the diagnosis of breast cancer. Acta Histochem. 118, 544-552 (2016).
30. A. G. Porter, R. U. Emerging roles of caspase-3 in apoptosis. Cell Death Differ. 6(2):99-104 (1999).
31. J. Felipe Lima, S. Nofech-Mozes, J. Bayani, J. M. Bartlett, EMT in breast carcinoma—a review. J Clin Med. 5, 65 (2016).
32. D. M. Richards, J. Hettinger, M. Feuerer, Monocytes and macrophages in cancer: development and functions. Cancer Microenviron. 6, 179-191 (2013).
33. Z. Fasoulakis, G. Kolios, V. Papamanolis, E. N. Kontomanolis, Interleukins associated with breast cancer. Cureus 10, e3549 (2018).
34. A. Masjedi et al., The significant role of interleukin-6 and its signaling pathway in the immunopathogenesis and treatment of breast cancer. Biomed Pharmacother. 108, 1415-1424 (2018).
35. R. Paolini, G. Bernardini, R. Molfetta, A. Santoni, NK cells and interferon. Cytokine Growth Factor Rev. 26, 113-120 (2015).
36. L. Shaashua et al., Perioperative COX-2 and β-Adrenergic Blockade Improves Metastatic Biomarkers in Breast Cancer Patients in a Phase-II Randomized Trial. Clin Cancer Re. 23, 4651-4661 (2017).s
37. A. Montoya et al., Use of non-selective β-blockers is associated with decreased tumor proliferative indices in early-stage breast cancer. Oncotarget 8, 6446-6460 (2017).
38. E. K. Sloan et al., The sympathetic nervous system induces a metastatic switch in primary breast cancer. Cancer Res. 70, 7042-7052 (2010)
39. S. Faulkner, P. Jobling, B. March, C. C. Jiang, H Hondermarck. Tumor Neurobiology and the War of Nerves in Cancer. Cancer Discov. 9, 702-710 (2019).
40. R. P. Xiao, S. Pepe, H. A. Spurgeon, M. C. Capogrossi, E. G. Lakatta, Opioid peptide receptor stimulation reverses beta-adrenergic effects in rat heart cells. Am. J. Physiol. 272, H797-805 (1997).
41. S. Bimonte et al., The effects of naloxone on human breast cancer progression: in vitro and in vivo studies on MDA.MB231 cells. Onco. Targets Ther. 11, 185-191 (2018).
42. M. M. Whalen, A. D. Bankhurst, Effects of beta-adrenergic receptor activation, cholera toxin and forskolin on human natural killer cell function. Biochem. J. 272, 327-331 (1990).
43. S. Ashrafi, R. Shapouri, A. Shirkhani, M. Mahdavi, Anti-tumor effects of propranolol: adjuvant activity on a transplanted murine breast cancer model. Biomed Pharmacother Biomedecine Pharmacother. 104, 45-51 (2018).
44. P. Pantziarka, B. A. Bryan, S. Crispino, E. B. Dickerson, Propranolol and breast cancer—a work in progress. Ecancermedicalscience 12, ed82 (2018).
45. M. Hollmén et al., G-CSF regulates macrophage phenotype and associates with poor overall survival in human triple-negative breast cancer. Oncoimmunology 5, e1115177 (2015).
46. P. Dutta, M. Sarkissyan, K. Paico, Y. Wu, J. V. Vadgama, MCP-1 is overexpressed in triple-negative breast cancers and drives cancer invasiveness and metastasis. Breast Cancer Res Treat. 70, 477-486 (2018).
47. L. I. Partecke et al., Chronic stress increases experimental pancreatic cancer growth, reduces survival, and can be antagonized by beta-adrenergic receptor blockade. Pancreatology 16, 423-433 (2016).
48. L. Parkitny, J. Younger, Reduced pro-inflammatory cytokines after eight weeks of low-dose naltrexone for fibromyalgia. Biomedicines 5, 16 (2017).
49. A. M. Abel, C. Yang, M. S. Thakar, S. Malarkannan, Natural killer cells: development, maturation, and clinical utilization. Front Immunol. 9, 1869 (2018).
50. A. Sica, Role of tumour-associated macrophages in cancer-related inflammation. Exp Oncol. 32, 153-158 (2010).

### REFERENCES (EXAMPLE 10)

51. Murugan, S.; Rousseau, B.; Sarkar, D. K. Beta 2 Adrenergic Receptor Antagonist Propranolol and Opioidergic Receptor Antagonist Naltrexone Produce Synergistic Effects on Breast Cancer Growth Prevention by Acting on Cancer Cells and Immune Environment in a Preclinical Model of Breast Cancer. Cancers 2021, 13, 4858.
52. Franken, N. A.; Rodermond, H. M.; Stap, J.; Haveman, J.; van Bree, C. Clonogenic assay of cells in vitro. Nat. Protoc. 2006, 1, 2315-2319.
53. Justus, C. R.; Leffler, N.; Ruiz-Echevarria, M.; Yang, L. V. In vitro cell migration and invasion assays. J. Vis. Exp. 2014, e51046.
54. C. P. Verschoor, A. Lelic, J. L. Brarnson, D. M. E. Bowdish, An introduction to automated flow cytometry gating tools and their implementation Front Immunol. 6:380 (2015).
   Exemplary compositions, methods and uses are set out in the following items:
   Item 1. A pharmaceutical composition, comprising:
      a. at least one MU (μ) opioid receptor (MOR) antagonist;
      b. at least one DELTA (Δ) opioid receptor (DOR) agonist;
      c. at least one BETA (β) 2-adrenergic receptor (B2AR) antagonist; and
      d. at least one pharmaceutically acceptable carrier or diluent.
   Item 2. The pharmaceutical composition according to item 1, wherein the at least one MOR antagonist is at least one oxymorphone.
   Item 3. The pharmaceutical composition according to item 2, wherein the at least one oxymorphone is selected from the group consisting of oxymorphone, naloxone, nalophrine, methylnaltrexone, alvimopan, naloxegol, naldemedine, naltrexone, methylnaltrexone, nalodeine, samidorphan, nalmefene, and cyclazocine.
   Item 4. The pharmaceutical composition according to item 2, wherein the at least one MOR antagonist has at least a 1:2 affinity (Kᵢ) for the μ-opioid receptor over the κ-opioid receptor.
   Item 5. The pharmaceutical composition according to item 2, wherein the at least one MOR antagonist has at least a 1:50 affinity (Kᵢ) for the μ-opioid receptor over the Δ-opioid receptor.
   Item 6. The pharmaceutical composition according to item 3, wherein the at least one oxymorphone is naltrexone.
   Item 7. The pharmaceutical composition according to item 1, wherein the at least one DOR agonist is a peptide selected from the group consisting of leu-enkephalin, met-enkephalin, deltorphin I, deltorphin II, (D-Pen², D-Pen⁵)-enkephalin (DPDPE), and (D-ALA², D-Leu⁵)-enkephalin (DADLE).
   Item 8. The pharmaceutical composition according to item 7, wherein the at least one peptide is DPDPE.
   Item 9. The pharmaceutical composition according to item 7, wherein the at least one peptide has greater than 80% binding at the Δ-opioid receptor.
   Item 10. The pharmaceutical composition according to item 1, wherein the at least one B2AR antagonist is a β-hydroxy amine containing compound.
   Item 11. The pharmaceutical composition according to item 10, wherein the at least one β-hydroxy amine containing compound is selected from the group consisting of IDI-118,551, bucindolol, butaxamine, carteolol, carvediol, labetalol, nadolol, oxprenolol, penbutolol, pindolol, propranolol, sotalol, and timolol.
   Item 12. The pharmaceutical composition according to item 11, wherein the at least one β-hydroxy amine containing compound is propranolol.
   Item 13. The pharmaceutical composition according to item 1, wherein the at least one MOR antagonist is present in an amount from 1 mg/kg to 20 mg/kg of body weight, preferably 1 to 10 mg/kg of body weight, and more preferably 10 mg/kg of body weight.
   Item 14. The pharmaceutical composition according to item 1, wherein the at least one DOR agonist is present in an amount from 50 μg/kg to 150 μg/kg of body weight, preferably 50 to 100 μg/kg of body weight, and more preferably 100 μg/kg of body weight.
   Item 15. The pharmaceutical composition according to item 1, wherein the at least one B2AR antagonist is present in an amount from 1 mg/kg to 20 mg/kg of body weight, preferably 1 to 10 mg/kg, and more preferably 10 mg/kg of body weight.
   Item 16. A pharmaceutical composition for affecting the growth and progression of breast cancers, comprising:
      a. at least one MU (μ) opioid receptor (MOR) antagonist;
      b. at least one DELTA (Δ) opioid receptor (DOR) agonist;
      c. at least one BETA (β) 2-adrenergic receptor (B2AR) antagonist; and
      d. at least one pharmaceutically acceptable carrier or diluent.
   Item 17. The pharmaceutical composition according to item 16, wherein the composition affects the growth and progression of breast cancer through at least one physiological pathway selected from the group consisting of increased cell growth arrest, elevated levels of apoptotic proteins, reduced production of epithelial-mesenchymal transition factors in tumor cells, increased innate immune activation, increased infiltration of NK cells in the tumor microenvironment, and the reduced inflammatory cytokine levels in plasma.
   Item 18. The pharmaceutical composition according to item 16, wherein the composition affects the growth and progression of at least one molecular subtype of breast cancer selected from the group consisting of luminal A, luminal B, triple-negative/basal-like, and Her2 type.
   Item 19. The pharmaceutical composition according to item 16, wherein the at least one MOR antagonist is naltrexone, the at least one DOR agonist is DPDPE, and the at least one B2AR antagonist is propranolol.
   Item 20. A method of treatment for affecting the growth and progression of a cancer in a patient in need thereof, comprising administering to the patient a therapeutically effective amount of a pharmaceutical composition, comprising:
      a. at least one MU (μ) opioid receptor (MOR) antagonist;
      b. at least one DELTA (Δ) opioid receptor (DOR) agonist;
      c. at least one BETA (β) 2-adrenergic receptor (B2AR) antagonist; and
      d. at least one pharmaceutically acceptable carrier or diluent.
   Item 21. The method of treatment of item 21, wherein the pharmaceutical composition comprises the pharmaceutical composition of item 2, 3, 4, 5, 6, 7, 8, 9, 10, 11, 12, 13, 14 or 15.
   Item 22. The pharmaceutical composition of item 1, 2, 3, 4, 5, 6, 7, 8, 9, 10, 11, 12, 13, 14 or 15 for use in a treatment for affecting the growth and progression of a cancer.
   Item 23. The pharmaceutical composition of item 16, 17, 18 or 19 for use in a treatment for affecting the growth and progression of breast cancer.

### Additional Exemplary Compositions, Methods and Uses are Set Out in the Following Items:

Item 1A. A pharmaceutical combination, comprising:
   a. at least one MU (μ) opioid receptor (MOR) antagonist;
   b. At least one BETA (β) 2-adrenergic receptor (B2AR) antagonist;
   c. at least one compound selected from the group consisting of:
      a. a DELTA (Δ) opioid receptor (DOR) agonist; and
      b. an immune checkpoint inhibitor (IC); and
   d. at least one pharmaceutically acceptable carrier or diluent.
Item 2A. The pharmaceutical combination according to item 1A, comprising:
   a. at least one MU (μ) opioid receptor (MOR) antagonist;
   b. at least one DELTA (Δ) opioid receptor (DOR) agonist;
   c. at least one BETA (β) 2-adrenergic receptor (B2AR) antagonist; and
   d. at least one pharmaceutically acceptable carrier or diluent.
Item 3A. The pharmaceutical combination according to item 1A, comprising:
   a. at least one MU (μ) opioid receptor (MOR) antagonist;
   b. at least one BETA (β) 2-adrenergic receptor (B2AR) antagonist;
   c. at least one immune checkpoint inhibitor (ICI); and
   d. at least one pharmaceutically acceptable carrier or diluent.
Item 4A. The pharmaceutical combination according to any one of items 1A-3A, wherein the at least one MOR antagonist is a 14-hydroxydihydromorphinone containing compound, or a salt thereof.
Item 5A. The pharmaceutical combination according to any one of items 1A-4A, wherein the at least one MOR antagonist is selected from the group consisting of naltrexone, oxymorphone, naloxone, nalophrine, methylnaltrexone, alvimopan, naloxol, naloxegol, naldemedine, nalodeine, samidorphan, nalmefene, levallorphan, nalbuphine, buprenorphine, diprenorphine, cyclazocine, and salts thereof.
Item 6A. The pharmaceutical combination according to any one of items 1A-5A, wherein the at least one MOR antagonist has at least a 1:2 affinity (Kᵢ) for the μ-opioid receptor over the κ-opioid receptor.
Item 7A. The pharmaceutical combination according to any one of items 1A-6A, wherein the at least one MOR antagonist has at least a 1:50 affinity (Kᵢ) for the μ-opioid receptor over the Δ-opioid receptor.
Item 8A. The pharmaceutical combination according to any one of items 1A-7A, wherein the at least one MOR antagonist is naltrexone, or a salt thereof.
Item 9A. The pharmaceutical combination according to item 1A or 2A, wherein the at least one DOR agonist is selected from the group consisting of (D-Pen², D-Pen⁵)-enkephalin (DPDPE), leu-enkephalin, met-enkephalin, deltorphin I, deltorphin 11, (D-ALA², D-Leu⁵)-enkephalin (DADLE), and salts thereof.
Item 10A. The pharmaceutical combination according to item 9A, wherein the at least one DOR agonist is DPDPE or a salt thereof.
Item 11A. The pharmaceutical combination according to item 9A, wherein the at least one DOR agonist has greater than 80% binding at the Δ-opioid receptor.
Item 12A. The pharmaceutical combination according to any one of items 1A-3A, wherein the at least one B2AR antagonist is a β-hydroxy amine containing compound, or a salt thereof.
Item 13A. The pharmaceutical combination according to item 12A, wherein the at least one β-hydroxy amine containing compound is selected from the group consisting of propranolol, IDI-118,551, bucindolol, butaxamine, carteolol, carvediol, labetalol, nadolol, oxprenolol, penbutolol, pindolol, sotalol, timolol, and salts thereof.
Item 14A. The pharmaceutical combination according to item 12A or 13A, wherein the at least one p3-hydroxy amine containing compound is propranolol, or a salt thereof.
Item 15A. The pharmaceutical combination according to item 1A or 3A, wherein the at least one ICI is selected from the group consisting of an anti-programmed cell death protein 1 (PD-1) inhibitor, an anti-programmed cell death protein 1 ligand (PD-L1) inhibitor, and a cytotoxic T lymphocyte antigen-4 (CTLA-4) inhibitor.
Item 16A. The pharmaceutical combination according to item 15A, wherein the at least one ICI is a PD-1 inhibitor.
Item 17A. The pharmaceutical combination according to item 16A, wherein the PD-1 inhibitor is an anti-PD1 antibody selected from the group consisting of nivolumab (Opdivo), pembrolizumab (Keytruda), cemiplimab (Libtayo), dostarlimab (Jemperli), JTX-4014, spartalizumab (PDR001), camrelizumab (SHR1210), sintilimab (1B1308), tislelizumab (BGB-A317), toripalimab (JS 001), pidilizumab, INCMGA00012 (MGA012), AMP-224, AMP-514, RMP1-14, and salts thereof.
Item 18A. The pharmaceutical combination according to any one of items 15A-17A, wherein the at least one ICI is nivolumab.
Item 19A. The pharmaceutical combination according to item 15A, wherein the at least one ICI is a PD-L1 inhibitor.
Item 20A. The pharmaceutical combination according to item 19A, wherein the PD-L1 inhibitor is an anti-PD-L1 antibody selected from the group consisting of atezolizumab (Tecentriq), avelumab (Bavencio), and durvalumab (Imfizi).
Item 21A. The pharmaceutical combination according to item 15A, wherein the at least one ICI is a CTLA-4 inhibitor.
Item 22A. The pharmaceutical combination according to item 21A, wherein CTLA-4 inhibitor is ipilimumab or tremelimumab.
Item 23A. The pharmaceutical combination according to any one of items 15A-22A, comprising two or more immune checkpoint inhibitors (ICIs).
Item 24A. The pharmaceutical combination according to item 23A, comprising a PD-1 inhibitor and a CTLA-4 inhibitor.
Item 25A. The pharmaceutical combination according to item 23A or 24A, comprising nivolumab and ipilimumab.
Item 26A. The pharmaceutical combination according to any one of items 1-A, wherein the at least one MOR antagonist is present in an amount from about 1 mg/kg to about 20 mg/kg of body weight, preferably from about 1 to about 10 mg/kg of body weight, and more preferably about 10 mg/kg of body weight.
Item 27A. The pharmaceutical combination according to any one of items 1A-3A, wherein the at least one B2AR antagonist is present in an amount from 1 mg/kg to 20 mg/kg of body weight, preferably 1 to 10 mg/kg, and more preferably 10 mg/kg of body weight.
Item 28A. The pharmaceutical combination according to any one of items 1A or 2A, wherein the at least one DOR agonist is present in an amount from about 50 μg/kg to about 150 μg/kg of body weight, preferably from about 50 to about 100 μg/kg of body weight, and more preferably about 100 μg/kg of body weight.
Item 29A. The pharmaceutical combination according to item 1A or 3A, wherein the at least one ICI is present in an amount from about 1 mg/kg to about 20 mg/kg of body weight, preferably from about 1 to about 5 mg/kg of body weight, and more preferably about 1 mg/kg or about 3 mg/kg of body weight.
Item 30A. The pharmaceutical combination according to any one of items 1A-29A, wherein the at least one MOR antagonist, DOR agonist and B2AR, antagonist or at least one MOR antagonist, B2AR antagonist and ICI are present in separate pharmaceutical compositions.
Item 31A. The pharmaceutical combination according to any one of items 1A-29A, wherein the at least one MOR antagonist, DOR and B2AR antagonist, or at least one MOR antagonist, B2AR antagonist and ICI are present in separate pharmaceutical compositions.
Item 32A. A pharmaceutical combination for use in a method of affecting growth and progression of cancer, the combination comprising:
   a. at least one MU (μ) opioid receptor (MOR) antagonist;
   b. at least one BETA (β) 2-adrenergic receptor (B2AR) antagonist;
   c. at least one compound selected from the group consisting of:
      i. a DELTA (Δ) opioid receptor (DOR) agonist; and
      ii. an immune checkpoint inhibitor (ICI); and
   d. at least one pharmaceutically acceptable carrier or diluent.
Item 33A. The pharmaceutical combination for use according to item 32A, wherein the cancer is selected from the group consisting of breast cancer, colorectal cancer, melanoma, infantile hemangiomas, glioblastoma, hepatocellular carcinoma, cutaneous squamous cell carcinoma, pancreatic cancer, oral cancer, and ovarian cancer.
Item 34A. The pharmaceutical combination for use according to item 32A or 33A, wherein the cancer is breast cancer.
Item 35A. The pharmaceutical combination for use according to item 32A or 33A, wherein the cancer is colorectal cancer.
Item 36A. The pharmaceutical combination for use according to item 32A or 33A, wherein the cancer is melanoma.
Item 37A. The pharmaceutical combination for use according to item 32A, the combination comprising:
   a. at least one MU (μ) opioid receptor (MOR) antagonist;
   b. at least one DELTA (Δ) opioid receptor (DOR) agonist;
   c. at least one BETA (β) 2-adrenergic receptor (B2AR) antagonist; and
   d. at least one pharmaceutically acceptable carrier or diluent.
Item 38A. The pharmaceutical combination for use according to item 37A, wherein the cancer is breast cancer.
Item 39A. The pharmaceutical combination for use according to item 38A, wherein the combination affects the growth and progression of breast cancer through at least one physiological pathway selected from the group consisting of increased cell growth arrest, elevated levels of apoptotic proteins, reduced production of epithelial-mesenchymal transition factors in tumor cells, increased innate immune activation, increased infiltration of NK cells in the tumor microenvironment, and the reduced inflammatory cytokine levels in plasma.
Item 40A. The pharmaceutical combination for use according to item 38A or 39A, wherein the combination affects the growth and progression of at least one molecular subtype of breast cancer selected from the group consisting of luminal A, luminal B, triple-negative/basal-like, and Her2 type.
Item 41A. The pharmaceutical combination for use according to any one of items 37A-40A, wherein the at least one MOR antagonist is naltrexone or a salt thereof, the at least one DOR agonist is DPDPE or a salt thereof, and the at least one B2AR antagonist is propranolol or a salt thereof.
Item 42A. The pharmaceutical combination according to any one of items 37A-41A, wherein the at least one MOR antagonist, DOR agonist and B2AR antagonist are present in separate pharmaceutical compositions.
Item 43A. The pharmaceutical combination according to any one of items 37A-41A, wherein the at least one MOR antagonist, DOR agonist and B2AR antagonist are present in the same pharmaceutical composition.
Item 44A. The pharmaceutical combination for use according to item 32A, the combination comprising:
   a. at least one MU (μ) opioid receptor (MOR) antagonist;
   b. at least one BETA (β) 2-adrenergic receptor (B2AR) antagonist;
   c. at least one immune checkpoint inhibitor (ICI); and
   d. at least one pharmaceutically acceptable carrier or diluent.
Item 45A. The pharmaceutical combination for use according to item 44, wherein the cancer is colorectal cancer or melanoma.
Item 46A. The pharmaceutical combination for use according to item 44A or 45A, wherein the at least one MOR antagonist is naltrexone, the at least one B2AR antagonist is propranolol, and the at least one ICI is ipilimumab and nivolumab.
Item 47A. The pharmaceutical combination according to any one of items 44A-46A, wherein the at least one MOR antagonist, B2AR antagonist and ICI are present in separate pharmaceutical compositions.
Item 48A. The pharmaceutical combination according to any one of items 4A4-46A, wherein the at least one MOR antagonist, B2AR antagonist and ICI are present in the same pharmaceutical composition.
Item 49A. A method of treatment for affecting the growth and progression of a cancer in a patient in need thereof, comprising administering to the patient a therapeutically effective amount of a pharmaceutical combination, the combination comprising:
   a. at least one MU (μ) opioid receptor (MOR) antagonist;
   b. At least one BETA (β) 2-adrenergic receptor (B2AR) antagonist;
   c. at least one compound selected from the group consisting of:
      i. a DELTA (Δ) opioid receptor (DOR) agonist; and
      ii. an immune checkpoint inhibitor (IC); and
         at least one pharmaceutically acceptable carrier or diluent.
Item 50A. The method according to item 49A, wherein the cancer is selected from the group consisting of breast cancer, colorectal cancer, melanoma, infantile hemangiomas, glioblastoma, hepatocellular carcinoma, cutaneous squamous cell carcinoma, pancreatic cancer, oral cancer, and ovarian cancer.
Item 51A. The method according to item 49A or 50A, wherein the cancer is breast cancer.
Item 52A. The method according to item 49A or 50A, wherein the cancer is colorectal cancer.
Item 53A. The method according to item 49A or 50A, wherein the cancer is melanoma.
Item 54A. The method according to item 49A, wherein the pharmaceutical combination comprises:
   e. at least one MU (μ) opioid receptor (MOR) antagonist;
   f. at least one DELTA (Δ) opioid receptor (DOR) agonist;
   g. at least one BETA (β) 2-adrenergic receptor (B2AR) antagonist; and
   h. at least one pharmaceutically acceptable carrier or diluent.
Item 55A. The method according to item 54A, wherein the cancer is breast cancer.
Item 56A. The method according to item 55A, wherein the combination affects the growth and progression of breast cancer through at least one physiological pathway selected from the group consisting of increased cell growth arrest, elevated levels of apoptotic proteins, reduced production of epithelial-mesenchymal transition factors in tumor cells, increased innate immune activation, increased infiltration of NK cells in the tumor microenvironment, and the reduced inflammatory cytokine levels in plasma.
Item 57A. The method according to item 55A or 56A, wherein the combination affects the growth and progression of at least one molecular subtype of breast cancer selected from the group consisting of luminal A, luminal B, triple-negative/basal-like, and Her2 type.
Item 58A. The method according to any one of items 54A-57A, wherein the at least one MOR antagonist is naltrexone, the at least one DOR agonist is DPDPE, and the at least one B2AR antagonist is propranolol.
Item 59A. The method according to item 49A, wherein the pharmaceutical combination comprises:
   a. at least one MU (μ) opioid receptor (MOR) antagonist;
   b. at least one BETA (β) 2-adrenergic receptor (B2AR) antagonist;
   c. at least one immune checkpoint inhibitor (ICI); and
   d. at least one pharmaceutically acceptable carrier or diluent.
Item 60A. The method according to item 59A, wherein the cancer is a colorectal cancer or melanoma.
Item 61A. The method according to item 59A or 60A, wherein the at least one MOR antagonist is naltrexone or a salt thereof, the at least one B2AR antagonist is propranolol or a salt thereof, and the at least one ICI comprises two ICI, wherein the two ICI are ipilimumab and nivolumab.
Item 62A. The method according to any one of items 59A-61A, wherein the at least one MOR antagonist, B2AR antagonist and ICI are present in separate pharmaceutical compositions.
Item 63A. The method according to any one of items 59A-61A, wherein the at least one MOR antagonist, B2AR antagonist and ICI are present in the same pharmaceutical composition.

1. A pharmaceutical combination, comprising:
a. at least one MU (μ) opioid receptor (MOR) antagonist;
b. at least one BETA (β) 2-adrenergic receptor (B2AR) antagonist;
c. at least one compound selected from the group consisting of:
a. a DELTA (Δ) opioid receptor (DOR) agonist; and
b. an immune checkpoint inhibitor (ICI); and
d. at least one pharmaceutically acceptable carrier or diluent.

2. The pharmaceutical combination according to claim 1, comprising:
a. at least one MU (μ) opioid receptor (MOR) antagonist;
b. at least one DELTA (Δ) opioid receptor (DOR) agonist;
c. at least one BETA (β) 2-adrenergic receptor (B2AR) antagonist; and
d. at least one pharmaceutically acceptable carrier or diluent.

3. The pharmaceutical combination according to claim 1, comprising:
a. at least one MU (μ) opioid receptor (MOR) antagonist;
b. at least one BETA (β) 2-adrenergic receptor (B2AR) antagonist;
c. at least one immune checkpoint inhibitor (ICI); and
d. at least one pharmaceutically acceptable carrier or diluent.

4. The pharmaceutical combination according to claim 1, wherein the at least one MOR antagonist is a 14-hydroxydihydromorphinone containing compound, or a salt thereof.

5. The pharmaceutical combination according to claim 1, wherein the at least one MOR antagonist is selected from the group consisting of naltrexone, oxymorphone, naloxone, nalophrine, methylnaltrexone, alvimopan, naloxol, naloxegol, naldemedine, nalodeine, samidorphan, nalmefene, levallorphan, nalbuphine, buprenorphine, diprenorphine, cyclazocine, and salts thereof.

6. The pharmaceutical combination according to claim 1, wherein the at least one MOR antagonist has at least a 1:2 affinity (Kᵢ) for the μ-opioid receptor over the κ-opioid receptor.

7. The pharmaceutical combination according to claim 1, wherein the at least one MOR antagonist has at least a 1:50 affinity (Kᵢ) for the μ-opioid receptor over the Δ-opioid receptor.

8. The pharmaceutical combination according to claim 1, wherein the at least one MOR antagonist is naltrexone, or a salt thereof.

9. The pharmaceutical combination according to claim 1, wherein the at least one DOR agonist is selected from the group consisting of (D-Pen², D-Pen⁵)-enkephalin (DPDPE), leu-enkephalin, met-enkephalin, deltorphin I, deltorphin II, (D-ALA², D-Leu⁵)-enkephalin (DADLE), and salts thereof.

10. The pharmaceutical combination according to claim 9, wherein the at least one DOR agonist is DPDPE or a salt thereof.

11. The pharmaceutical combination according to claim 9, wherein the at least one DOR agonist has greater than 80% binding at the Δ-opioid receptor.

12. The pharmaceutical combination according to claim 1, wherein the at least one B2AR antagonist is a β-hydroxy amine containing compound, or a salt thereof.

13. The pharmaceutical combination according to claim 12, wherein the at least one β-hydroxy amine containing compound is selected from the group consisting of propranolol, IDI-118,551, bucindolol, butaxamine, carteolol, carvediol, labetalol, nadolol, oxprenolol, penbutolol, pindolol, sotalol, timolol, and salts thereof.

14. The pharmaceutical combination according to claim 12, wherein the at least one β-hydroxy amine containing compound is propranolol, or a salt thereof.

15. The pharmaceutical combination according to claim 1, wherein the at least one ICI is selected from the group consisting of an anti-programmed cell death protein 1 (PD-1) inhibitor, an anti-programmed cell death protein 1 ligand (PD-L1) inhibitor, and a cytotoxic T lymphocyte antigen-4 (CTLA-4) inhibitor.

16. The pharmaceutical combination according to claim 15, wherein the at least one ICI is a PD-1 inhibitor.

17. The pharmaceutical combination according to claim 16, wherein the PD-1 inhibitor is an anti-PD1 antibody selected from the group consisting of nivolumab (Opdivo), pembrolizumab (Keytruda), cemiplimab (Libtayo), dostarlimab (Jemperli), JTX-4014, spartalizumab (PDR001), camrelizumab (SHR1210), sintilimab (1B1308), tislelizumab (BGB-A317), toripalimab (JS 001), pidilizumab, INCMGA00012 (MGA012), AMP-224, AMP-514, RMP1-14, and salts thereof.

18. The pharmaceutical combination according to claim 15, wherein the at least one ICI is nivolumab.

19. The pharmaceutical combination according to claim 15, wherein the at least one ICI is a PD-L1 inhibitor.

20. The pharmaceutical combination according to claim 19, wherein the PD-L1 inhibitor is an anti-PD-L1 antibody selected from the group consisting of atezolizumab (Tecentriq), avelumab (Bavencio), and durvalumab (Imfizi).

21. The pharmaceutical combination according to claim 15, wherein the at least one ICI is a CTLA-4 inhibitor.

22. The pharmaceutical combination according to claim 21, wherein CTLA-4 inhibitor is ipilimumab or tremelimumab.

23. The pharmaceutical combination according to claim 15, comprising two or more immune checkpoint inhibitors (ICIs).

24. The pharmaceutical combination according to claim 23, comprising a PD-1 inhibitor and a CTLA-4 inhibitor.

25. The pharmaceutical combination according to claim 23, comprising nivolumab and ipilimumab.

26. The pharmaceutical combination according to claim 1, wherein the at least one MOR antagonist is present in an amount from about 1 mg/kg to about 20 mg/kg of body weight, preferably from about 1 to about 10 mg/kg of body weight, and more preferably about 10 mg/kg of body weight.

27. The pharmaceutical combination according to claim 1, wherein the at least one B2AR antagonist is present in an amount from 1 mg/kg to 20 mg/kg of body weight, preferably 1 to 10 mg/kg, and more preferably 10 mg/kg of body weight.

28. The pharmaceutical combination according to claim 1, wherein the at least one DOR agonist is present in an amount from about 50 μg/kg to about 150 μg/kg of body weight, preferably from about 50 to about 100 μg/kg of body weight, and more preferably about 100 μg/kg of body weight.

29. The pharmaceutical combination according to claim 1, wherein the at least one ICI is present in an amount from about 1 mg/kg to about 20 mg/kg of body weight, preferably from about 1 to about 5 mg/kg of body weight, and more preferably about 1 mg/kg or about 3 mg/kg of body weight.

30. The pharmaceutical combination according to claim 1, wherein the at least one MOR antagonist, DOR agonist and B2AR antagonist, or at least one MOR antagonist, B2AR antagonist and ICI are present in separate pharmaceutical compositions.

31. The pharmaceutical combination according to claim 1, wherein the at least one MOR antagonist, DOR agonist and B2AR antagonist, or at least one MOR antagonist, B2AR antagonist and ICI are present in separate pharmaceutical compositions.

32-41. (canceled)

42. A method of treatment for affecting the growth and progression of a cancer in a patient in need thereof, comprising administering to the patient a therapeutically effective amount of a pharmaceutical combination, the combination comprising:
a. at least one MU (μ) opioid receptor (MOR) antagonist;
b. at least one BETA (β) 2-adrenergic receptor (B2AR) antagonist;
c. at least one compound selected from the group consisting of:
i. a DELTA (Δ) opioid receptor (DOR) agonist; and
ii. an immune checkpoint inhibitor (ICI); and
d. at least one pharmaceutically acceptable carrier or diluent.

43. The method according to claim 42, wherein the pharmaceutical combination comprises:
a. at least one MU (μ) opioid receptor (MOR) antagonist;
b. at least one DELTA (Δ) opioid receptor (DOR) agonist;
c. at least one BETA (β) 2-adrenergic receptor (B2AR) antagonist; and
d. at least one pharmaceutically acceptable carrier or diluent.

44. The method according to claim 42, wherein the pharmaceutical combination comprises:
a. at least one MU (μ) opioid receptor (MOR) antagonist;
b. at least one BETA (β) 2-adrenergic receptor (B2AR) antagonist;
c. at least one immune checkpoint inhibitor (ICI); and
d. at least one pharmaceutically acceptable carrier or diluent.
